(19) United States
(12) Patent Application Publication
(10) Pub. No.: US 2025/0277194 A1
(43) Pub. Date: Sep. 4, 2025

(54) METHODS AND COMPOSITIONS FOR SHORT STATURE PLANTS THROUGH MANIPULATION OF GIBBERELLIN METABOLISM

(71) Applicant: Monsanto Technology LLC, St. Louis, MO (US)

(72) Inventors: Edwards ALLEN, O'Fallon, MO (US); Jayanand BODDU, St. Louis, MO (US); Alexander GOLDSHMIDT, Rehovot (IL); Anil NEELAM, Wildwood, MO (US); Tomasz PACIOREK, Wildwood, MO (US); Thomas SLEWINSKI, Chesterfield, MO (US); Huai WANG, Chesterfield, MO (US)

(73) Assignee: Monsanto Technology LLC, St. Louis, MO (US)

(21) Appl. No.: 18/267,061

(22) PCT Filed: Dec. 15, 2021
(86) PCT No.: PCT/US2021/072940

§ 371 Date: Jun. 13, 2023

Related U.S. Application Data
(60) Provisional application No. 63/125,752, filed on Dec. 15, 2020.
(60) Provisional application No. 63/180,344, filed on Apr. 27, 2021.

(51) Int. Cl. C12N 9/02; C12N 15/82

(52) U.S. Cl. CPC C12N 9/0071; C12N 15/8225; C12N 15/8262; C12N 15/8297; C12Y 114/11013

(57) ABSTRACT

The present disclosure provides compositions and methods for altering gibberellin (GA) content in corn plants. Methods and compositions are provided for ectopically or transgenically expressing a GA2 oxidase in corn plants. Modified plants, plant parts and plant cells having a recombinant DNA construct for expression of a GA2 oxidase transgene are further provided that may comprise reduced gibberellin levels and improved characteristics, such as reduced plant height and increased lodging resistance, but without off-types.

### CROSS-REFERENCE TO RELATED APPLICATIONS

This application is a national stage application under 35 U.S.C. § 371 of International Application No. PCT/US2021/072940, filed internationally on Dec. 15, 2021, which claims priority to and the benefit of U.S. Provisional Application No. 63/125,752, filed on Dec. 15, 2020 and U.S. Provisional Application No. 63/180,344, filed on Apr. 27, 2021, the entire disclosures of each of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 777052061200SEQLIST.TXT, date recorded: Jun. 6, 2023, size: 732,209 bytes).

### BACKGROUND

### Field

The present disclosure relates to compositions and methods for improving traits, such as lodging resistance and increased yield in corn plants.

### Related Art

Gibberellins (gibberellic acids or GAs) are plant hormones that regulate a number of major plant growth and developmental processes. Manipulation of GA levels in semi-dwarf wheat, rice and Sorghum plant varieties led to increased yield and reduced lodging in these cereal crops during the 20^{th} century, which was largely responsible for the Green Revolution. However, successful yield gains in other cereal crops, such as corn, have not been realized through manipulation of the GA pathway. Indeed, some mutations in the GA pathway genes have been associated with various off-types in corn that are incompatible with yield, which has led researchers away from finding semi-dwarf, high-yielding corn varieties via manipulation of the GA pathway.

There continues to be a need in the art for the development of additional traits in corn plants, for increased yield and/or resistance to lodging.

### SUMMARY

In one aspect, provided herein is a recombinant DNA construct comprising a transcribable DNA sequence encoding a GA2 oxidase protein and a plant-expressible promoter, wherein the transcribable DNA sequence is operably linked to the plant-expressible promoter. In some embodiments of this aspect, the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 2, 4, 6, 8, 10, 12, 14, 16, 18, 20, 22, 24 and/or 26. In some embodiments of this aspect, which may be combined with the preceding embodiment, the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 1, 3, 5, 7, 9, 11, 13, 15, 17, 19, 21, 23 and/or 25. In some embodiments of this aspect, which may be combined with any of the preceding embodiments, the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 28, 30, 32, 34, 36, 38, 40, 42, 44 and/or 46. In some embodiments of this aspect, which may be combined with any of the preceding embodiments, the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 27, 29, 31, 33, 35, 37, 39, 41, 43 and/or 45. In some embodiments of this aspect, which may be combined with any of the preceding embodiments, the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 48, 50, 52, 54, 56, 58, 60 and/or 62. In some embodiments of this aspect, which may be combined with any of the preceding embodiments, the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 47, 49, 51, 53, 55, 57, 59 and/or 61. In some embodiments of this aspect, which may be combined with any of the preceding embodiments, the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 64, 66, 68, 70, 72, 74, 76, 78, 80, 82, 84, 86, 88, 90, 92 and/or 94. In some embodiments of this aspect, which may be combined with any of the preceding embodiments, the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 63, 65, 67, 69, 71, 73, 75, 77, 79, 81, 83, 85, 87, 89, 91 and/or 93. In some embodiments of this aspect, which may be combined with any of the preceding embodiments, the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 96, 98, 100, 102, 104, 106, 108, 110, 112, 114, 116, 118, 120, 122 and/or 124. In some embodiments of this aspect, which may be combined with any of the preceding embodiments, the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 95, 97, 99, 101, 103, 105, 107, 109, 111, 113, 115, 117, 119, 121 and/or 123. In some embodiments of this aspect, which may be combined with any of the preceding embodiments, the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 126, 128, 130, 132, 134, 136, 138, 140, 142, 144 and/or 146. In some embodiments of this aspect, which may be combined with any of the preceding embodiments, the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 125, 127, 129, 131, 133, 135, 137, 139, 141, 143 and/or 145. In some embodiments of this aspect, which may be combined with any of the preceding embodiments, the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 148, 150, 152, 154, 156, 158, 160, 162, 164, 166, 168, 170, 172, 174, 176 and/or 178. In some embodiments of this aspect, which may be combined with any of the preceding embodiments, the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 147, 149, 151, 153, 155, 157, 159, 161, 163, 165, 167, 169, 171, 173, 175 and/or 177. In some embodiments of this aspect, which may be combined with any of the preceding embodiments, the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 180, 182, 184, 186, 188, 190, 192, 194, 196, 198, 200, 202, 204, 206 and/or 208. In some embodiments of this aspect, which may be combined with any of the preceding embodiments, the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 179, 181, 183, 185, 187, 189, 191, 193, 195, 197, 199, 201, 203, 205 and/or 207. In some embodiments of this aspect, which may be combined with any of the preceding embodiments, the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 210, 212, 214, 216, 218, 220, 222, 224, 226, 228, 230, 232, 234, 236 and/or 238. In some embodiments of this aspect, which may be combined with any of the preceding embodiments, the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 209, 211, 213, 215, 217, 219, 221, 223, 225, 227, 229, 231, 233, 235 and/or 237. In some embodiments of this aspect, which may be combined with any of the preceding embodiments, the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 240, 242, 244, 246, 248, 250 and/or 252. In some embodiments of this aspect, which may be combined with any of the preceding embodiments, the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 239, 241, 243, 245, 247, 249 and/or 251. In some embodiments of this aspect, which may be combined with any of the preceding embodiments, the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 254, 256, 258, 260, 262, 264 and/or 266. In some embodiments of this aspect, which may be combined with any of the preceding embodiments, the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 253, 255, 257, 259, 261, 263 and/or 265. In some embodiments of this aspect, which may be combined with any of the preceding embodiments, the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 268, 270, 272, 274, 276, 278, 280, 282 and/or 284. In some embodiments of this aspect, which may be combined with any of the preceding embodiments, the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 267, 269, 271, 273, 275, 277, 279, 281 and/or 283. In some embodiments of this aspect, which may be combined with any of the preceding embodiments, the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 286, 288, 290 and/or 292. In some embodiments of this aspect, which may be combined with any of the preceding embodiments, the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 285, 287, 289 and/or 291. In some embodiments of this aspect, which may be combined with any of the preceding embodiments, the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 294, 296, 298, 300, 302, 304, 306 and/or 308. In some embodiments of this aspect, which may be combined with any of the preceding embodiments, the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 293, 295, 297, 299, 301, 303, 305 and/or 307. In some embodiments of this aspect, which may be combined with any of the preceding embodiments, the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 310, 312, 314, 316, 318, 320 and/or 322. In some embodiments of this aspect, which may be combined with any of the preceding embodiments, the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 309, 311, 313, 315, 317, 319 and/or 321. In some embodiments of this aspect, which may be combined with any of the preceding embodiments, the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 324 and/or 326. In some embodiments of this aspect, which may be combined with any of the preceding embodiments, the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 323 and/or 325. In some embodiments of this aspect, which may be combined with any of the preceding embodiments, the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 328, 330 and/or 332. In some embodiments of this aspect, which may be combined with any of the preceding embodiments, the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 327, 329 and/or 331.

In some embodiments of this aspect, which may be combined with any of the preceding embodiments, the plant-expressible promoter is a vascular promoter. In some embodiments, the vascular promoter comprises one of the following: a sucrose synthase promoter, a sucrose transporter promoter, a Sh1 promoter, Commelina yellow mottle virus (CoYMV) promoter, a wheat dwarf geminivirus (WDV) large intergenic region (LIR) promoter, a maize streak geminivirus (MSV) coat protein (CP) promoter, a rice yellow stripe 1 (YS1)-like promoter, or a rice yellow stripe 2 (OsYSL2) promoter. In certain embodiments, the vascular promoter comprises a DNA sequence that is at least 80%, at least 85%, at least 90%, at least 95%, at least 96%, at least 97%, at least 98%, at least 99%, at least 99.5% or 100% identical to one or more of SEQ ID NO: 335, SEQ ID NO: 336, SEQ ID NO: 337, SEQ ID NO: 338, or SEQ ID NO: 339, or a functional portion thereof.

In some embodiments of this aspect, which may be combined with any of the preceding embodiments, the plant-expressible promoter is a RTBV promoter. In certain embodiments wherein the plant-expressible promoter is a RTBV promoter, the plant-expressible promoter comprises a DNA sequence that is at least 80%, at least 85%, at least 90%, at least 95%, at least 96%, at least 97%, at least 98%, at least 99%, at least 99.5% or 100% identical to one or more of SEQ ID NO: 333 or SEQ ID NO: 334, or a functional portion thereof.

In some embodiments of this aspect, which may be combined with any of the preceding embodiments, the plant-expressible promoter is a leaf promoter. In some embodiments, the leaf promoter comprises one of the following: a RuBisCO promoter, a PPDK promoter, a FDA promoter, a Nadh-Gogat promoter, a chlorophyll a/b binding protein gene promoter, a phosphoenolpyruvate carboxylase (PEPC) promoter, or a Myb gene promoter. In certain embodiments, the leaf promoter comprises a DNA sequence that is at least 80%, at least 85%, at least 90%, at least 95%, at least 96%, at least 97%, at least 98%, at least 99%, at least 99.5% or 100% identical to one or more of SEQ ID NO: 340, SEQ ID NO: 341 or SEQ ID NO: 342, or a functional portion thereof.

In some embodiments of this aspect, which may be combined with any of the preceding embodiments, the plant-expressible promoter is a constitutive promoter. In some embodiments, the constitutive promoter is selected from the group consisting of: an actin promoter, a CaMV 35S or 19S promoter, a plant ubiquitin promoter, a plant Gos2 promoter, a FMV promoter, a CMV promoter, a MMV promoter, a PCLSV promoter, an Emu promoter, a tubulin promoter, a nopaline synthase promoter, an octopine synthase promoter, a mannopine synthase promoter, or a maize alcohol dehydrogenase, or a functional portion thereof. In certain embodiments, the constitutive promoter comprises a DNA sequence that is at least 80%, at least 85%, at least 90%, at least 95%, at least 96%, at least 97%, at least 98%, at least 99%, at least 99.5% or 100% identical to one or more of SEQ ID NOs: 343, SEQ ID NO: 344, SEQ ID NO: 345, SEQ ID NO: 346, SEQ ID NO: 347, SEQ ID NO: 348, SEQ ID NO: 349, SEQ ID NO: 350 or SEQ ID NO: 351, or a functional portion thereof.

In another aspect, provided herein is a transformation vector comprising the recombinant DNA construct of any one of the preceding embodiments.

In yet another aspect, provided herein is a transgenic corn plant, plant part or plant cell comprising the recombinant DNA construct of any one of the preceding embodiments stably integrated into the genome of the transgenic corn plant, plant part or plant cell. In some embodiments of this aspect, the transgenic corn plant has one or more of the following traits relative to a control plant: shorter plant height, increased stalk/stem diameter, improved lodging resistance, reduced green snap, deeper roots, increased leaf area, earlier canopy closure, higher stomatal conductance, lower ear height, increased foliar water content, improved drought tolerance, improved nitrogen use efficiency, reduced anthocyanin content and area in leaves under normal or nitrogen-limiting or water-limiting stress conditions, increased ear weight, increased harvest index, increased yield, increased seed number, increased seed weight, and/or increased prolificacy. In some embodiments of this aspect, which may be combined with any one of the preceding embodiments, the transgenic corn plant has a shorter plant height and/or improved lodging resistance. In some embodiments of this aspect, which may be combined with any one of the preceding embodiments, the height of the transgenic corn plant is at least 10%, at least 20%, at least 25%, at least 30%, at least 35%, or at least 40% shorter than a wild-type control plant. In some embodiments of this aspect, which may be combined with any one of the preceding embodiments, the stalk or stem diameter of the transgenic corn plant at one or more stem internodes is at least 5%, at least 10%, at least 15%, at least 20%, at least 25%, at least 30%, at least 35%, or at least 40% greater than the stalk or stem diameter at the same one or more internodes of a wild-type control plant. In some embodiments of this aspect, which may be combined with any one of the preceding embodiments, the stalk or stem diameter of the transgenic corn plant at one or more of the first, second, third, and/or fourth internode below the ear is at least 5%, at least 10%, at least 15%, at least 20%, at least 25%, at least 30%, at least 35%, or at least 40% greater than the same internode of a wild-type control plant. In some embodiments of this aspect, which may be combined with any one of the preceding embodiments, the level of one or more active GAs in at least one internode tissue of the stem or stalk of the transgenic corn plant is lower than the same internode tissue of a wild-type control plant. In some embodiments of this aspect, which may be combined with any one of the preceding embodiments, the level of one or more active GAs in at least one internode tissue of the stem or stalk of the transgenic corn plant is at least 5%, at least 10%, at least 15%, at least 20%, at least 25%, at least 30%, at least 35%, or at least 40% lower than the same internode tissue of a wild-type control plant. In some embodiments of this aspect, which may be combined with any one of the preceding embodiments, the transgenic corn plant does not have any significant off-types in at least one female organ or ear.

In still another aspect, provided herein is a bacterial or host cell comprising recombinant DNA construct of any one of the preceding embodiments.

In yet another aspect, provided herein is a method for producing a transgenic corn plant, comprising: (a) transforming at least one cell of an explant with recombinant DNA construct of any one of the preceding embodiments, and (b) regenerating or developing the transgenic corn plant comprising the recombinant DNA construct from the transformed explant. In some embodiments of this aspect, the at least one cell of an explant is transformed via Agrobacterium mediated transformation or particle bombardment. In some embodiments of this aspect, the at least one cell of an explant is transformed via site-directed integration using a targeted genome editing technique. In some embodiments, the at least one cell of an explant is transformed using a recombinant DNA donor template comprising at least one homology arm and an insertion sequence, wherein the at least one homology arm is complementary to a target site in the genome of a corn plant, wherein the insertion sequence comprises a recombinant DNA construct comprising a transcribable DNA sequence encoding a GA2 oxidase mRNA and protein, wherein the transcribable DNA sequence is operably linked to a plant-expressible promoter, and wherein the insertion sequence comprising the recombinant DNA construct is stably integrated into the genome of the transgenic corn plant. In certain embodiments, the at least one homology arm is complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a target site in the genome of a corn plant. In some embodiments, the at least one homology arm is at least 70%, at least 75%, at least 80%, at least 85%, at least 90%, at least 95%, at least 96%, at least 97%, at least 99% or 100% complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a target site in the genome of a corn plant. In some embodiments of this aspect, which may be combined with any of the preceding embodiments, the at least one cell of an explant is transformed using a site-specific nuclease. In some embodiments, the site-specific nuclease is a meganuclease, a zinc-finger nuclease (ZFN), a RNA-guided endonuclease, a TALE-endonuclease (TALEN), a recombinase, or a transposase. In certain embodiments, the site-specific nuclease is an RNA-guided endonuclease. In some embodiments, the at least one cell of an explant is further transformed using a guide RNA (gRNA) molecule. In some embodiments, the RNA-guided endonuclease in association with the guide RNA molecule causes a double strand break or nick at or near the target DNA sequence of the guide RNA in the genome of the corn plant to direct the integration of the insertion sequence into the genome of the transgenic corn plant at or near the target DNA sequence of the guide RNA. In certain embodiments, the guide RNA molecule is a CRISPR RNA (crRNA) or a single-chain guide RNA (sgRNA). In some embodiments, the guide RNA comprises a sequence complementary to a protospacer adjacent motif (PAM) sequence present in the genome of the transgenic corn plant immediately adjacent to the target DNA sequence. In some embodiments of this aspect, which may be combined with any of the preceding embodiments, target site of the homology arm in the genome of a corn plant is at or near the target site or target DNA sequence of the site-specific nuclease and/or the guide RNA. In some embodiments of this aspect, which may be combined with any of the preceding embodiments, the method further comprises (c) selecting a transgenic corn plant comprising the recombinant DNA construct. In some embodiments, the selecting step (c) comprises determining if the recombinant DNA construct was transformed or integrated into the genome of the transgenic corn plant using a molecular assay. In other embodiments, the selecting step (c) comprises determining if the recombinant DNA construct was transformed or integrated into the genome of the transgenic corn plant by observing a plant phenotype.

In still another aspect, provided herein is a recombinant DNA donor template comprising at least one homology arm and an insertion sequence, wherein the at least one homology arm is at least 70%, at least 75%, at least 80%, at least 85%, at least 90%, at least 95%, at least 96%, at least 97%, at least 99% or 100% complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a target site in the genome of a corn plant, wherein the insertion sequence comprises a recombinant DNA construct comprising a transcribable DNA sequence encoding a GA2 oxidase mRNA and protein and a plant expressible promoter, and wherein the transcribable DNA sequence is operably linked to the plant-expressible promoter. which may be combined with any of the preceding embodiments, the at least one homology arm comprises two homology arms including a first homology arm and a second homology arm, wherein the first homology arm comprises a sequence that is at least 70%, at least 75%, at least 80%, at least 85%, at least 90%, at least 95%, at least 96%, at least 97%, at least 99% or 100% complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a first flanking DNA sequence, and the second homology arm comprises a sequence that is at least 70%, at least 75%, at least 80%, at least 85%, at least 90%, at least 95%, at least 96%, at least 97%, at least 99% or 100% complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a second flanking DNA sequence, wherein the first flanking DNA sequence and the second flanking DNA sequence are genomic sequences at or near the same genomic locus of a corn plant, and wherein the insertion sequence is located between the first homology arm and the second homology arm.

In yet another aspect, provided herein is a composition comprising the recombinant DNA donor template of any one of the embodiments of the preceding aspect. In still another aspect, provided herein is a composition comprising the recombinant DNA donor template of any one of the embodiments of the preceding aspects and a site-specific nuclease. In some embodiments of this aspect, the composition further comprises a guide RNA.

In still another aspect, provided herein is a DNA molecule or vector comprising the recombinant DNA donor template of any one of the embodiments of the preceding aspect. In some embodiments of this aspect, the DNA molecule or vector further comprises a polynucleotide sequence encoding a site-specific nuclease. In some embodiments of this aspect, which may be combined with any of the preceding embodiments, the DNA molecule or vector further comprises a polynucleotide sequence encoding a guide RNA.

In yet another aspect, provided herein is a composition comprising the DNA molecule or vector of any one of the embodiments of the preceding aspect. In still another aspect, provided herein is a composition comprising the DNA molecule or vector of any one of the embodiments of the preceding aspects and a site-specific nuclease. In some embodiments of this aspect, the composition further comprises a guide RNA.

In still another aspect, provided herein is a composition comprising a first DNA molecule or vector and a second DNA molecule or vector, wherein the first DNA molecule or vector comprises the recombinant DNA donor template of any one of the embodiments of the preceding aspects, and the second DNA molecule or vector comprises a polynucleotide sequence encoding a site-specific nuclease. In some embodiments of this aspect, the first DNA molecule or vector or the second DNA molecule or vector comprises a polynucleotide sequence encoding a guide RNA.

In yet another aspect, provided herein is a composition comprising a first DNA molecule or vector and a second DNA molecule or vector, wherein the first DNA molecule or vector comprises the recombinant DNA donor template of any one of the embodiments of the preceding aspects, and the second DNA molecule or vector comprises a polynucleotide sequence encoding a guide RNA.

In still another aspect, provided herein is a transgenic corn plant, plant part or plant cell comprising the insertion sequence of the recombinant DNA donor template of any one of the embodiments of the preceding aspects.

Certain aspects of the disclosure include:

Aspect A1: A recombinant DNA construct comprising a transcribable DNA sequence encoding a GA2 oxidase protein and a plant-expressible promoter, wherein the transcribable DNA sequence is operably linked to the plant-expressible promoter.

Aspect A2: The recombinant DNA construct of Aspect A1, wherein the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 2, 4, 6, 8, 10, 12, 14, 16, 18, 20, 22, 24 and/or 26.

Aspect A3: The recombinant DNA construct of Aspect A1 or A2, wherein the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 1, 3, 5, 7, 9, 11, 13, 15, 17, 19, 21, 23 and/or 25.

Aspect A4: The recombinant DNA construct of any one of Aspects A1-A3, wherein the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 28, 30, 32, 34, 36, 38, 40, 42, 44 and/or 46.

Aspect A5: The recombinant DNA construct of any one of Aspects A1-A4, wherein the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 27, 29, 31, 33, 35, 37, 39, 41, 43 and/or 45.

Aspect A6: The recombinant DNA construct of any one of Aspects A1-A5, wherein the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 48, 50, 52, 54, 56, 58, 60 and/or 62.

Aspect A7: The recombinant DNA construct of any one of Aspects A1-A6, wherein the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 47, 49, 51, 53, 55, 57, 59 and/or 61.

Aspect A8: The recombinant DNA construct of any one of Aspects A1-A7, wherein the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 64, 66, 68, 70, 72, 74, 76, 78, 80, 82, 84, 86, 88, 90, 92 and/or 94.

Aspect A9: The recombinant DNA construct of any one of Aspects A1-A8, wherein the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 63, 65, 67, 69, 71, 73, 75, 77, 79, 81, 83, 85, 87, 89, 91 and/or 93.

Aspect A10: The recombinant DNA construct of any one of Aspects A1-A9, wherein the plant-expressible promoter is a vascular promoter.

Aspect A11: The recombinant DNA construct of Aspect A10, wherein the vascular promoter comprises one of the following: a sucrose synthase promoter, a sucrose transporter promoter, a Sh1 promoter, Commelina yellow mottle virus (CoYMV) promoter, a wheat dwarf geminivirus (WDV) large intergenic region (LIR) promoter, a maize streak geminivirus (MSV) coat protein (CP) promoter, a rice yellow stripe 1 (YS1)-like promoter, or a rice yellow stripe 2 (OsYSL2) promoter.

Aspect A12: The recombinant DNA construct of Aspect A10, wherein the vascular promoter comprises a DNA sequence that is at least 80%, at least 85%, at least 90%, at least 95%, at least 96%, at least 97%, at least 98%, at least 99%, at least 99.5% or 100% identical to one or more of SEQ ID NO: 335, SEQ ID NO: 336, SEQ ID NO: 337, SEQ ID NO: 338, or SEQ ID NO: 339, or a functional portion thereof.

Aspect A13: The recombinant DNA construct of any one of Aspects A1-A10, wherein the plant-expressible promoter is a RTBV promoter.

Aspect A14: The recombinant DNA construct of Aspect A13, wherein the plant-expressible promoter comprises a DNA sequence that is at least 80%, at least 85%, at least 90%, at least 95%, at least 96%, at least 97%, at least 98%, at least 99%, at least 99.5% or 100% identical to one or more of SEQ ID NO: 333 or SEQ ID NO: 334, or a functional portion thereof.

Aspect A15: The recombinant DNA construct of any one of Aspects A1-A9, wherein the plant-expressible promoter is a leaf promoter.

Aspect A16: The recombinant DNA construct of Aspect A15, wherein the leaf promoter comprises one of the following: a RuBisCO promoter, a PPDK promoter, a FDA promoter, a Nadh-Gogat promoter, a chlorophyll a/b binding protein gene promoter, a phosphoenolpyruvate carboxylase (PEPC) promoter, or a Myb gene promoter.

Aspect A17: The recombinant DNA construct of Aspect A15, wherein the leaf promoter comprises a DNA sequence that is at least 80%, at least 85%, at least 90%, at least 95%, at least 96%, at least 97%, at least 98%, at least 99%, at least 99.5% or 100% identical to one or more of SEQ ID NO: 340, SEQ ID NO: 341 or SEQ ID NO: 342, or a functional portion thereof.

Aspect A18: The recombinant DNA construct of any one of Aspects A1-A9, wherein the plant-expressible promoter is a constitutive promoter.

Aspect A19: The recombinant DNA construct of Aspect A18, wherein the constitutive promoter is selected from the group consisting of: an actin promoter, a CaMV 35S or 19S promoter, a plant ubiquitin promoter, a plant Gos2 promoter, a FMV promoter, a CMV promoter, a MMV promoter, a PCLSV promoter, an Emu promoter, a tubulin promoter, a nopaline synthase promoter, an octopine synthase promoter, a mannopine synthase promoter, or a maize alcohol dehydrogenase, or a functional portion thereof.

Aspect A20: The recombinant DNA construct of Aspect A18, wherein the constitutive promoter comprises a DNA sequence that is at least 80%, at least 85%, at least 90%, at least 95%, at least 96%, at least 97%, at least 98%, at least 99%, at least 99.5% or 100% identical to one or more of SEQ ID NOs: 343, SEQ ID NO: 344, SEQ ID NO: 345, SEQ ID NO: 346, SEQ ID NO: 347, SEQ ID NO: 348, SEQ ID NO: 349, SEQ ID NO: 350 or SEQ ID NO: 351, or a functional portion thereof.

Aspect A21: A transformation vector comprising the recombinant DNA construct of any one of Aspects A1-A20.

Aspect A22: A transgenic corn plant, plant part or plant cell comprising the recombinant DNA construct of any one of Aspects A1-A20 stably integrated into the genome of the transgenic corn plant, plant part or plant cell.

Aspect A23: The transgenic corn plant of Aspect A22, wherein the transgenic corn plant has one or more of the following traits relative to a control plant: shorter plant height, increased stalk/stem diameter, improved lodging resistance, reduced green snap, deeper roots, increased leaf area, earlier canopy closure, higher stomatal conductance, lower ear height, increased foliar water content, improved drought tolerance, improved nitrogen use efficiency, reduced anthocyanin content and area in leaves under normal or nitrogen-limiting or water-limiting stress conditions, increased ear weight, increased harvest index, increased yield, increased seed number, increased seed weight, and/or increased prolificacy.

Aspect A24: The transgenic corn plant of Aspect A22 or A23, wherein the transgenic corn plant has a shorter plant height and/or improved lodging resistance.

Aspect A25: The transgenic corn plant of any one of Aspects A22-A24, wherein the height of the transgenic corn plant is at least 10%, at least 20%, at least 25%, at least 30%, at least 35%, or at least 40% shorter than a wild-type control plant.

Aspect A26: The transgenic corn plant of any one of Aspects A22-A25, wherein the stalk or stem diameter of the transgenic corn plant at one or more stem internodes is at least 5%, at least 10%, at least 15%, at least 20%, at least 25%, at least 30%, at least 35%, or at least 40% greater than the stalk or stem diameter at the same one or more internodes of a wild-type control plant.

Aspect A27: The transgenic corn plant of any one of Aspects A22-A26, wherein the stalk or stem diameter of the transgenic corn plant at one or more of the first, second, third, and/or fourth internode below the ear is at least 5%, at least 10%, at least 15%, at least 20%, at least 25%, at least 30%, at least 35%, or at least 40% greater than the same internode of a wild-type control plant.

Aspect A28: The transgenic corn plant of any one of Aspects A22-A27, wherein the level of one or more active GAs in at least one internode tissue of the stem or stalk of the transgenic corn plant is lower than the same internode tissue of a wild-type control plant.

Aspect A29: The transgenic corn plant of any one of Aspects A22-A28, wherein the level of one or more active GAs in at least one internode tissue of the stem or stalk of the transgenic corn plant is at least 5%, at least 10%, at least 15%, at least 20%, at least 25%, at least 30%, at least 35%, or at least 40% lower than the same internode tissue of a wild-type control plant.

Aspect A30: The transgenic corn plant of any one of Aspects A22-A29, wherein the transgenic corn plant does not have any significant off-types in at least one female organ or ear.

Aspect A31: A bacterial or host cell comprising the recombinant DNA construct or vector of any one of Aspects A1-A21.

Aspect A32: A method for producing a transgenic corn plant, comprising: (a) transforming at least one cell of an explant with the recombinant DNA construct or vector of any one of Aspects A1-A21, and (b) regenerating or developing the transgenic corn plant comprising the recombinant DNA construct from the transformed explant.

Aspect A33: The method of Aspect A32, wherein the at least one cell of an explant is transformed via Agrobacterium mediated transformation or particle bombardment.

Aspect A34: The method of Aspect A32, wherein the at least one cell of an explant is transformed via site-directed integration using a targeted genome editing technique.

Aspect A35: The method of Aspect A34, wherein the at least one cell of an explant is transformed using a recombinant DNA donor template comprising at least one homology arm and an insertion sequence, wherein the at least one homology arm is complementary to a target site in the genome of a corn plant, wherein the insertion sequence comprises a recombinant DNA construct comprising a transcribable DNA sequence encoding a GA2 oxidase mRNA and protein, wherein the transcribable DNA sequence is operably linked to a plant-expressible promoter, and wherein the insertion sequence comprising the recombinant DNA construct is stably integrated into the genome of the transgenic corn plant.

Aspect A36: The method of Aspect A35, wherein the at least one homology arm is complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a target site in the genome of a corn plant.

Aspect A37: The method of Aspect A35 or A36, wherein the at least one homology arm is at least 70%, at least 75%, at least 80%, at least 85%, at least 90%, at least 95%, at least 96%, at least 97%, at least 99% or 100% complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a target site in the genome of a corn plant.

Aspect A38: The method of any one of Aspects A33-A37, wherein the at least one cell of an explant is transformed using a site-specific nuclease.

Aspect A39: The method of Aspect A38, wherein the site-specific nuclease is a meganuclease, a zinc-finger nuclease (ZFN), a RNAguided endonuclease, a TALE-endonuclease (TALEN), a recombinase, or a transposase.

Aspect A40: The method of Aspect A38, wherein the site-specific nuclease is an RNAguided endonuclease.

Aspect A41: The method of Aspect A40, wherein the at least one cell of an explant is further transformed using a guide RNA (gRNA) molecule.

Aspect A42: The method of Aspect A41, wherein the RNAguided endonuclease in association with the guide RNA molecule causes a double strand break or nick at or near the target DNA sequence of the guide RNA in the genome of the corn plant to direct the integration of the insertion sequence into the genome of the transgenic corn plant at or near the target DNA sequence of the guide RNA

Aspect A43: The method of Aspect A41 or A42, wherein the guide RNA molecule is a CRISPR RNA (crRNA) or a single-chain guide RNA (sgRNA).

Aspect A44: The method of any one of Aspects A41-A43, wherein the guide RNA comprises a sequence complementary to a protospacer adjacent motif (PAM) sequence present in the genome of the transgenic corn plant immediately adjacent to the target DNA sequence.

Aspect A45: The method of any one of Aspects A35-A44, wherein target site of the homology arm in the genome of a corn plant is at or near the target site or target DNA sequence of the site-specific nuclease and/or the guide RNA.

Aspect A46: The method of any one of Aspects A32-A45, further comprising: (c) selecting a transgenic corn plant comprising the recombinant DNA construct.

Aspect A47: The method of Aspect A46, wherein the selecting step (c) comprises determining if the recombinant DNA construct was transformed or integrated into the genome of the transgenic corn plant using a molecular assay.

Aspect A48: The method of Aspect A46, wherein the selecting step (c) comprises determining if the recombinant DNA construct was transformed or integrated into the genome of the transgenic corn plant by observing a plant phenotype.

Aspect A49: A recombinant DNA donor template comprising at least one homology arm and an insertion sequence, wherein the at least one homology arm is at least 70%, at least 75%, at least 80%, at least 85%, at least 90%, at least 95%, at least 96%, at least 97%, at least 99% or 100% complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a target site in the genome of a corn plant, wherein the insertion sequence comprises a recombinant DNA construct comprising a transcribable DNA sequence encoding a GA2 oxidase mRNA and protein and a plant expressible promoter, and wherein the transcribable DNA sequence is operably linked to the plant-expressible promoter.

Aspect A50: The recombinant DNA donor template of Aspect A49, wherein the at least one homology arm comprises two homology arms including a first homology arm and a second homology arm, wherein the first homology arm comprises a sequence that is at least 70%, at least 75%, at least 80%, at least 85%, at least 90%, at least 95%, at least 96%, at least 97%, at least 99% or 100% complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a first flanking DNA sequence, and the second homology arm comprises a sequence that is at least 70%, at least 75%, at least 80%, at least 85%, at least 90%, at least 95%, at least 96%, at least 97%, at least 99% or 100% complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a second flanking DNA sequence, wherein the first flanking DNA sequence and the second flanking DNA sequence are genomic sequences at or near the same genomic locus of a corn plant, and wherein the insertion sequence is located between the first homology arm and the second homology arm.

Aspect A51: A DNA molecule or vector comprising the recombinant DNA donor template of Aspect A49 or A50.

Aspect A52: The DNA molecule or vector of Aspect A51, further comprising a polynucleotide sequence encoding a site-specific nuclease.

Aspect A53: The DNA molecule or vector of Aspect A51 or A52, further comprising a polynucleotide sequence encoding a guide RNA.

Aspect A54: A composition comprising the DNA molecule or vector and/or the recombinant DNA donor template of any one of Aspects A49-A53 and a guide RNA.

Aspect A55: A composition comprising the DNA molecule or vector and/or the recombinant DNA donor template of any one of Aspects A49-A53 and a site-specific nuclease.

Aspect A56: The composition of Aspect A55, further comprising a guide RNA.

Aspect A57: A composition comprising a first DNA molecule or vector and a second DNA molecule or vector, wherein the first DNA molecule or vector comprises the recombinant DNA donor template of Aspect A49 or A50 and the second DNA molecule or vector comprises a polynucleotide sequence encoding a site-specific nuclease.

Aspect A58: The composition of Aspect A57, wherein the first DNA molecule or vector or the second DNA molecule or vector comprises a polynucleotide sequence encoding a guide RNA.

Aspect A59: A composition comprising a first DNA molecule or vector and a second DNA molecule or vector, wherein the first DNA molecule or vector comprises the recombinant DNA donor template of Aspect A49 or A50 and the second DNA molecule or vector comprises a polynucleotide sequence encoding a guide RNA.

Aspect A60: A transgenic corn plant, plant part or plant cell comprising the insertion sequence of the recombinant DNA donor template of any one of Aspects A49-A53.

Aspect B1: A recombinant DNA construct comprising a transcribable DNA sequence encoding a GA2 oxidase protein and a plant-expressible promoter, wherein the transcribable DNA sequence is operably linked to the plant-expressible promoter.

Aspect B2: The recombinant DNA construct of Aspect B1, wherein the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 2, 4, 6, 8, 10, 12, 14, 16, 18, 20, 22, 24 and/or 26.

Aspect B3: The recombinant DNA construct of Aspect B1 or B2, wherein the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 1, 3, 5, 7, 9, 11, 13, 15, 17, 19, 21, 23 and/or 25.

Aspect B4: The recombinant DNA construct of any one of Aspects B1-B3, wherein the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 28, 30, 32, 34, 36, 38, 40, 42, 44 and/or 46.

Aspect B5: The recombinant DNA construct of any one of Aspects B1-B4, wherein the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 27, 29, 31, 33, 35, 37, 39, 41, 43 and/or 45.

Aspect B6: The recombinant DNA construct of any one of Aspects B1-B5, wherein the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 48, 50, 52, 54, 56, 58, 60 and/or 62.

Aspect B7: The recombinant DNA construct of any one of Aspects B1-B6, wherein the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 47, 49, 51, 53, 55, 57, 59 and/or 61.

Aspect B8: The recombinant DNA construct of any one of Aspects 1B-B7, wherein the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 64, 66, 68, 70, 72, 74, 76, 78, 80, 82, 84, 86, 88, 90, 92 and/or 94.

Aspect B9: The recombinant DNA construct of any one of Aspects B1-B8, wherein the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 63, 65, 67, 69, 71, 73, 75, 77, 79, 81, 83, 85, 87, 89, 91 and/or 93.

Aspect B10: The recombinant DNA construct of any one of Aspects B1-B9, wherein the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 96, 98, 100, 102, 104, 106, 108, 110, 112, 114, 116, 118, 120, 122 and/or 124.

Aspect B11: The recombinant DNA construct of any one of Aspects B1-B10, wherein the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 95, 97, 99, 101, 103, 105, 107, 109, 111, 113, 115, 117, 119, 121 and/or 123.

Aspect B12: The recombinant DNA construct of any one of Aspects B1-B11, wherein the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 126, 128, 130, 132, 134, 136, 138, 140, 142, 144 and/or 146.

Aspect B13: The recombinant DNA construct of any one of Aspects B1-B12, wherein the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 125, 127, 129, 131, 133, 135, 137, 139, 141, 143 and/or 145.

Aspect B14: The recombinant DNA construct of any one of Aspects B1-B13, wherein the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 148, 150, 152, 154, 156, 158, 160, 162, 164, 166, 168, 170, 172, 174, 176 and/or 178.

Aspect B15: The recombinant DNA construct of any one of Aspects B1-B14, wherein the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 147, 149, 151, 153, 155, 157, 159, 161, 163, 165, 167, 169, 171, 173, 175 and/or 177.

Aspect B16: The recombinant DNA construct of any one of Aspects B1-B15, wherein the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 180, 182, 184, 186, 188, 190, 192, 194, 196, 198, 200, 202, 204, 206 and/or 208.

Aspect B17: The recombinant DNA construct of any one of Aspects B1-B16, wherein the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 179, 181, 183, 185, 187, 189, 191, 193, 195, 197, 199, 201, 203, 205 and/or 207.

Aspect B18: The recombinant DNA construct of any one of Aspects B1-B17, wherein the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 210, 212, 214, 216, 218, 220, 222, 224, 226, 228, 230, 232, 234, 236 and/or 238.

Aspect B19: The recombinant DNA construct of any one of Aspects B1-B18, wherein the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 209, 211, 213, 215, 217, 219, 221, 223, 225, 227, 229, 231, 233, 235 and/or 237.

Aspect B20: The recombinant DNA construct of any one of Aspects B1-B19, wherein the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 240, 242, 244, 246, 248, 250 and/or 252.

Aspect B21: The recombinant DNA construct of any one of Aspects 1-20, wherein the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 239, 241, 243, 245, 247, 249 and/or 251.

Aspect B22: The recombinant DNA construct of any one of Aspects B1-B21, wherein the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 254, 256, 258, 260, 262, 264 and/or 266.

Aspect B23: The recombinant DNA construct of any one of Aspects B1-B22, wherein the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 253, 255, 257, 259, 261, 263 and/or 265.

Aspect B24: The recombinant DNA construct of any one of Aspects B1-B23, wherein the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 268, 270, 272, 274, 276, 278, 280, 282 and/or 284.

Aspect B25: The recombinant DNA construct of any one of Aspects B1-B24, wherein the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 267, 269, 271, 273, 275, 277, 279, 281 and/or 283.

Aspect B26: The recombinant DNA construct of any one of Aspects B1-B25, wherein the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 286, 288, 290 and/or 292.

Aspect B27: The recombinant DNA construct of any one of Aspects B1-B26, wherein the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 285, 287, 289 and/or 291.

Aspect B28: The recombinant DNA construct of any one of Aspects B1-B27, wherein the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 294, 296, 298, 300, 302, 304, 306 and/or 308.

Aspect B29: The recombinant DNA construct of any one of Aspects B1-B28, wherein the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 293, 295, 297, 299, 301, 303, 305 and/or 307.

Aspect B30: The recombinant DNA construct of any one of Aspects B1-B29, wherein the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 310, 312, 314, 316, 318, 320 and/or 322.

Aspect B31: The recombinant DNA construct of any one of Aspects B1-B30, wherein the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 309, 311, 313, 315, 317, 319 and/or 321.

Aspect B32: The recombinant DNA construct of any one of Aspects B1-B31, wherein the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 324 and/or 326.

Aspect B33: The recombinant DNA construct of any one of Aspects B1-B32, wherein the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 323 and/or 325.

Aspect B34: The recombinant DNA construct of any one of Aspects B1-B33, wherein the GA2 oxidase protein is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 328, 330 and/or 332.

Aspect B35: The recombinant DNA construct of any one of Aspects B1-B34, wherein the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 327, 329 and/or 331.

Aspect B36: The recombinant DNA construct of any one of Aspects B1-B35, wherein the plant-expressible promoter is a vascular promoter.

Aspect B37: The recombinant DNA construct of Aspect B36, wherein the vascular promoter comprises one of the following: a sucrose synthase promoter, a sucrose transporter promoter, a Sh1 promoter, Commelina yellow mottle virus (CoYMV) promoter, a wheat dwarf geminivirus (WDV) large intergenic region (LIR) promoter, a maize streak geminivirus (MSV) coat protein (CP) promoter, a rice yellow stripe 1 (YS1)-like promoter, or a rice yellow stripe 2 (OsYSL2) promoter.

Aspect B38: The recombinant DNA construct of Aspect B36, wherein the vascular promoter com-prises a DNA sequence that is at least 80%, at least 85%, at least 90%, at least 95%, at least 96%, at least 97%, at least 98%, at least 99%, at least 99.5% or 100% identical to one or more of SEQ ID NO: 335, SEQ ID NO: 336, SEQ ID NO: 337, SEQ ID NO: 338, or SEQ ID NO: 339, or a functional portion thereof.

Aspect B39: The recombinant DNA construct of any one of Aspects B1-B38, wherein the plant-expressible promoter is a RTBV promoter.

Aspect B40: The recombinant DNA construct of Aspect 39, wherein the plant-expressible promoter comprises a DNA sequence that is at least 80%, at least 85%, at least 90%, at least 95%, at least 96%, at least 97%, at least 98%, at least 99%, at least 99.5% or 100% identical to one or more of SEQ ID NO: 333 or SEQ ID NO: 334, or a functional portion thereof.

Aspect B41: The recombinant DNA construct of any one of Aspects B1-B35, wherein the plant-expressible promoter is a leaf promoter.

Aspect B42: The recombinant DNA construct of Aspect B41, wherein the leaf promoter comprises one of the following: a RuBisCO promoter, a PPDK promoter, a FDA promoter, a Nadh-Gogat promoter, a chlorophyll a/b binding protein gene promoter, a phosphoenolpyruvate carboxylase (PEPC) promoter, or a Myb gene promoter.

Aspect B43: The recombinant DNA construct of Aspect B41, wherein the leaf promoter comprises a DNA sequence that is at least 80%, at least 85%, at least 90%, at least 95%, at least 96%, at least 97%, at least 98%, at least 99%, at least 99.5% or 100% identical to one or more of SEQ ID NO: 340, SEQ ID NO: 341 or SEQ ID NO: 342, or a functional portion thereof.

Aspect B44: The recombinant DNA construct of any one of Aspects B1-B35, wherein the plant-expressible promoter is a constitutive promoter.

Aspect B45: The recombinant DNA construct of Aspect B44, wherein the constitutive promoter is selected from the group consisting of: an actin promoter, a CaMV 35S or 19S pro-moter, a plant ubiquitin promoter, a plant Gos2 promoter, a FMV promoter, a CMV promoter, a MMV promoter, a PCLSV promoter, an Emu promoter, a tubulin pro-moter, a nopaline synthase promoter, an octopine synthase promoter, a mannopine synthase promoter, or a maize alcohol dehydrogenase, or a functional portion there-of.

Aspect B46: The recombinant DNA construct of Aspect B44, wherein the constitutive promoter comprises a DNA sequence that is at least 80%, at least 85%, at least 90%, at least 95%, at least 96%, at least 97%, at least 98%, at least 99%, at least 99.5% or 100% identical to one or more of SEQ ID NOs: 343, SEQ ID NO: 344, SEQ ID NO: 345, SEQ ID NO: 346, SEQ ID NO: 347, SEQ ID NO: 348, SEQ ID NO: 349, SEQ ID NO: 350 or SEQ ID NO: 351, or a functional portion thereof.

Aspect B47: A transformation vector comprising the recombinant DNA construct of any one of Aspects B1-B46.

Aspect B48: A transgenic corn plant, plant part or plant cell comprising the recombinant DNA construct of any one of Aspects B1-B46 stably integrated into the genome of the trans-genic corn plant, plant part or plant cell.

Aspect B49: The transgenic corn plant of Aspect B48, wherein the transgenic corn plant has one or more of the following traits relative to a control plant: shorter plant height, increased stalk/stem diameter, improved lodging resistance, reduced green snap, deeper roots, increased leaf area, earlier canopy closure, higher stomatal conductance, lower ear height, increased foliar water content, improved drought tolerance, improved nitro-gen use efficiency, reduced anthocyanin content and area in leaves under normal or nitrogen-limiting or water-limiting stress conditions, increased ear weight, increased harvest index, increased yield, increased seed number, increased seed weight, and/or increased prolificacy.

Aspect B50: The transgenic corn plant of Aspect B48 or B49, wherein the transgenic corn plant has a shorter plant height and/or improved lodging resistance.

Aspect B51: The transgenic corn plant of any one of Aspects B48-B50, wherein the height of the transgenic corn plant is at least 10%, at least 20%, at least 25%, at least 30%, at least 35%, or at least 40% shorter than a wild-type control plant.

Aspect B52: The transgenic corn plant of any one of Aspects B48-B51, wherein the stalk or stem diameter of the transgenic corn plant at one or more stem internodes is at least 5%, at least 10%, at least 15%, at least 20%, at least 25%, at least 30%, at least 35%, or at least 40% greater than the stalk or stem diameter at the same one or more internodes of a wild-type control plant.

Aspect B53: The transgenic corn plant of any one of Aspects B48-B52, wherein the stalk or stem diameter of the transgenic corn plant at one or more of the first, second, third, and/or fourth internode below the ear is at least 5%, at least 10%, at least 15%, at least 20%, at least 25%, at least 30%, at least 35%, or at least 40% greater than the same inter-node of a wild-type control plant.

Aspect B54: The transgenic corn plant of any one of Aspects B48-B53, wherein the level of one or more active GAs in at least one internode tissue of the stem or stalk of the transgenic corn plant is lower than the same internode tissue of a wild-type control plant.

Aspect B55: The transgenic corn plant of any one of Aspects B48-B54, wherein the level of one or more active GAs in at least one internode tissue of the stem or stalk of the transgenic corn plant is at least 5%, at least 10%, at least 15%, at least 20%, at least 25%, at least 30%, at least 35%, or at least 40% lower than the same internode tissue of a wild-type control plant.

Aspect B56: The transgenic corn plant of any one of Aspects B48-B55, wherein the transgenic corn plant does not have any significant off-types in at least one female organ or ear.

Aspect B57: A bacterial or host cell comprising the recombinant DNA construct or vector of any one of Aspects B1-B46.

Aspect B58: A method for producing a transgenic corn plant, comprising: (a) transforming at least one cell of an explant with the recombinant DNA construct or vector of any one of Aspects B1-B46, and (b) regenerating or developing the transgenic corn plant comprising the recombinant DNA construct from the transformed explant.

Aspect B59: The method of Aspect B58, wherein the at least one cell of an explant is transformed via Agrobacterium mediated transformation or particle bombardment.

Aspect B60: The method of Aspect B58, wherein the at least one cell of an explant is transformed via site-directed integration using a targeted genome editing technique.

Aspect B61: The method of Aspect B60, wherein the at least one cell of an explant is transformed using a recombinant DNA donor template comprising at least one homology arm and an insertion sequence, wherein the at least one homology arm is complementary to a target site in the genome of a corn plant, wherein the insertion sequence com-prises a recombinant DNA construct comprising a transcribable DNA sequence encoding a GA2 oxidase mRNA and protein, wherein the transcribable DNA sequence is operably linked to a plant-expressible promoter, and wherein the insertion se-quence comprising the recombinant DNA construct is stably integrated into the genome of the transgenic corn plant.

Aspect B62: The method of Aspect B61, wherein the at least one homology arm is complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a target site in the genome of a corn plant.

Aspect B63: The method of Aspect B61 or B62, wherein the at least one homology arm is at least 70%, at least 75%, at least 80%, at least 85%, at least 90%, at least 95%, at least 96%, at least 97%, at least 99% or 100% complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a target site in the genome of a corn plant.

Aspect B64: The method of any one of Aspects B58-B63, wherein the at least one cell of an explant is transformed using a site-specific nuclease.

Aspect B65: The method of Aspect B64, wherein the site-specific nuclease is a meganuclease, a zinc-finger nuclease (ZFN), a RNA-guided endonuclease, a TALE-endonuclease (TALEN), a recombinase, or a transposase.

Aspect B66: The method of Aspect B64, wherein the site-specific nuclease is an RNA-guided endo-nuclease.

Aspect B67: The method of Aspect B66, wherein the at least one cell of an explant is further trans-formed using a guide RNA (gRNA) molecule.

Aspect B68: The method of Aspect B67, wherein the RNA-guided endonuclease in association with the guide RNA molecule causes a double strand break or nick at or near the target DNA sequence of the guide RNA in the genome of the corn plant to direct the integration of the insertion sequence into the genome of the transgenic corn plant at or near the target DNA sequence of the guide RNA

Aspect B69: The method of Aspect B67 or B68, wherein the guide RNA molecule is a CRISPR RNA (crRNA) or a single-chain guide RNA (sgRNA).

Aspect B70: The method of any one of Aspects B67-B69, wherein the guide RNA comprises a sequence complementary to a protospacer adjacent motif (PAM) sequence present in the genome of the transgenic corn plant immediately adjacent to the target DNA sequence.

Aspect B71: The method of any one of Aspects B61-B70, wherein target site of the homology arm in the genome of a corn plant is at or near the target site or target DNA sequence of the site-specific nuclease and/or the guide RNA.

Aspect B72: The method of any one of Aspects B58-B71, further comprising: (c) selecting a transgenic corn plant comprising the recombinant DNA construct.

Aspect B73: The method of Aspect B72, wherein the selecting step (c) comprises determining if the recombinant DNA construct was transformed or integrated into the genome of the transgenic corn plant using a molecular assay.

Aspect B74: The method of Aspect B72, wherein the selecting step (c) comprises determining if the recombinant DNA construct was transformed or integrated into the genome of the transgenic corn plant by observing a plant phenotype.

Aspect B75: A recombinant DNA donor template comprising at least one homology arm and an insertion sequence, wherein the at least one homology arm is at least 70%, at least 75%, at least 80%, at least 85%, at least 90%, at least 95%, at least 96%, at least 97%, at least 99% or 100% complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a target site in the genome of a corn plant, wherein the insertion sequence comprises a recombinant DNA construct comprising a transcribable DNA sequence encoding a GA2 oxidase mRNA and protein and a plant expressible promoter, and wherein the transcribable DNA sequence is operably linked to the plant-expressible promoter.

Aspect B76: The recombinant DNA donor template of Aspect B75, wherein the at least one homology arm comprises two homology arms including a first homology arm and a second homology arm, wherein the first homology arm comprises a sequence that is at least 70%, at least 75%, at least 80%, at least 85%, at least 90%, at least 95%, at least 96%, at least 97%, at least 99% or 100% complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a first flanking DNA sequence, and the second homology arm comprises a sequence that is at least 70%, at least 75%, at least 80%, at least 85%, at least 90%, at least 95%, at least 96%, at least 97%, at least 99% or 100% complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a second flanking DNA sequence, wherein the first flanking DNA sequence and the second flanking DNA se-quence are genomic sequences at or near the same genomic locus of a corn plant, and wherein the insertion sequence is located between the first homology arm and the second homology arm.

Aspect B77: A DNA molecule or vector comprising the recombinant DNA donor template of Aspect B75 or B76.

Aspect B78: The DNA molecule or vector of Aspect B77, further comprising a polynucleotide se-quence encoding a site-specific nuclease.

Aspect B79: The DNA molecule or vector of Aspect B77 or B78, further comprising a polynucleotide sequence encoding a guide RNA.

Aspect B80: A composition comprising the DNA molecule or vector and/or the recombinant DNA donor template of any one of Aspects B75-B79 and a guide RNA.

Aspect B81: A composition comprising the DNA molecule or vector and/or the recombinant DNA donor template of any one of Aspects B75-B79 and a site-specific nuclease.

Aspect B82: The composition of Aspect B81, further comprising a guide RNA.

Aspect B83: A composition comprising a first DNA molecule or vector and a second DNA molecule or vector, wherein the first DNA molecule or vector comprises the recombinant DNA donor template of Aspect B75 or B76 and the second DNA molecule or vector comprises a polynucleotide sequence encoding a site-specific nuclease.

Aspect B84: The composition of Aspect B83, wherein the first DNA molecule or vector or the second DNA molecule or vector comprises a polynucleotide sequence encoding a guide RNA.

Aspect B85: A composition comprising a first DNA molecule or vector and a second DNA molecule or vector, wherein the first DNA molecule or vector comprises the recombinant DNA donor template of Aspect B75 or B76 and the second DNA molecule or vector comprises a polynucleotide sequence encoding a guide RNA.

Aspect B86: A transgenic corn plant, plant part or plant cell comprising the insertion sequence of the recombinant DNA donor template of any one of Aspects B75-B79.

### BRIEF DESCRIPTION OF THE DRAWINGS

FIG. 1 shows a representative image of a transgenic corn plant at V9 stage that is homozygous for Event 2 for overexpression of GA2 oxidase_3 (GA2Ox3) transgene, next to a wild-type control plant.

FIG. 2 shows a bar graph of the average plant heights at V5 or V9 stage of inbred corn plants homozygous for multiple events of each transgene expressing either GA2 oxidase_3 (GA2Ox3) (Events 1-3), GA2 oxidase_9 (GA2Ox9) (Events 4-6), or GA2 oxidase_2 (GA2Ox2) (Events 7-9), relative to wild-type control plants.

FIG. 3 shows a bar graph of the average plant heights at V5 or V9 stage of inbred corn plants hemizygous for multiple events of each transgene expressing either GA2 oxidase_3 (GA2Ox3) (Event 1), GA2 oxidase_9 (GA2Ox9) (Events 4 and 6), or GA2 oxidase_2 (GA2Ox2) (Events 7 and 8), relative to wild-type control plants.

FIG. 4 shows a bar graph of the average plant heights at V5 or V9 stage of hybrid corn plants hemizygous for multiple events of the GA2 oxidase_2 (GA2Ox2) (Events 7-9), relative to wild-type control plants.

### DETAILED DESCRIPTION

### Definitions

To facilitate understanding of the disclosure, several terms and abbreviations as used herein are defined below as follows:

The term “and/or” when used in a list of two or more items, means that any one of the listed items can be employed by itself or in combination with any one or more of the listed items. For example, the expression “A and/or B” is intended to mean either or both of A and B —i.e., A alone, B alone, or A and B in combination. The expression “A, B and/or C” is intended to mean A alone, B alone, C alone, A and B in combination, A and C in combination, B and C in combination, or A, B, and C in combination.

The term “about” as used herein, is intended to qualify the numerical values that it modifies, denoting such a value as variable within a margin of error. When no particular margin of error, such as a standard deviation to a mean value, is recited, the term “about” should be understood to mean that range which would encompass the recited value and the range which would be included by rounding up or down to that figure, taking into account significant figures.

The term “cereal plant” as used herein refers a monocotyledonous (monocot) crop plant that is in the Poaceae or Gramineae family of grasses and is typically harvested for its seed, including, for example, wheat, corn, rice, millet, barley, Sorghum, oat and rye. As commonly understood, a “corn plant” or “maize plant” refers to any plant of species Zea mays and includes all plant varieties that can be bred with corn, including wild maize species.

The terms “percent identity” or “percent identical” as used herein in reference to two or more nucleotide or protein sequences is calculated by (i) comparing two optimally aligned sequences (nucleotide or protein) over a window of comparison, (ii) determining the number of positions at which the identical nucleic acid base (for nucleotide sequences) or amino acid residue (for proteins) occurs in both sequences to yield the number of matched positions, (iii) dividing the number of matched positions by the total number of positions in the window of comparison, and then (iv) multiplying this quotient by 100% to yield the percent identity. For purposes of calculating “percent identity” between DNA and RNA sequences, a uracil (U) of a RNA sequence is considered identical to a thymine (T) of a DNA sequence. If the window of comparison is defined as a region of alignment between two or more sequences (i.e., excluding nucleotides at the 5′ and 3′ ends of aligned polynucleotide sequences, or amino acids at the N-terminus and C-terminus of aligned protein sequences, that are not identical between the compared sequences), then the “percent identity” may also be referred to as a “percent alignment identity”. If the “percent identity” is being calculated in relation to a reference sequence without a particular comparison window being specified, then the percent identity is determined by dividing the number of matched positions over the region of alignment by the total length of the reference sequence. Accordingly, for purposes of the present disclosure, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%.

It is recognized that residue positions of proteins that are not identical often differ by conservative amino acid substitutions, where amino acid residues are substituted for other amino acid residues with similar size and chemical properties (e.g., charge, hydrophobicity, polarity, etc.), and therefore may not change the functional properties of the molecule. When sequences differ in conservative substitutions, the percent sequence similarity may be adjusted upwards to correct for the conservative nature of the non-identical substitution(s). Sequences that differ by such conservative substitutions are said to have “sequence similarity” or “similarity.” Thus, “percent similarity” or “percent similar” as used herein in reference to two or more protein sequences is calculated by (i) comparing two optimally aligned protein sequences over a window of comparison, (ii) determining the number of positions at which the same or similar amino acid residue occurs in both sequences to yield the number of matched positions, (iii) dividing the number of matched positions by the total number of positions in the window of comparison (or the total length of the reference or query protein if a window of comparison is not specified), and then (iv) multiplying this quotient by 100% to yield the percent similarity. Conservative amino acid substitutions for proteins are known in the art.

For optimal alignment of sequences to calculate their percent identity or similarity, various pair-wise or multiple sequence alignment algorithms and programs are known in the art, such as ClustalW, or Basic Local Alignment Search Tool® (BLAST®), etc., that may be used to compare the sequence identity or similarity between two or more nucleotide or protein sequences. Although other alignment and comparison methods are known in the art, the alignment between two sequences (including the percent identity ranges described above) may be as determined by the ClustalW or BLAST® algorithm, see, e.g., Chenna R. et al., “Multiple sequence alignment with the Clustal series of programs,” Nucleic Acids Research 31: 3497-3500 (2003); Thompson J D et al., “Clustal W: Improving the sensitivity of progressive multiple sequence alignment through sequence weighting, position-specific gap penalties and weight matrix choice,” Nucleic Acids Research 22: 4673-4680 (1994); and Larkin M A et al., “Clustal W and Clustal X version 2.0,” Bioinformatics 23: 2947-48 (2007); and Altschul, S. F., Gish, W., Miller, W., Myers, E. W. & Lipman, D. J. (1990) “Basic local alignment search tool.” J. Mol. Biol. 215:403-410 (1990), the entire contents and disclosures of which are incorporated herein by reference.

The terms “percent complementarity” or “percent complementary”, as used herein in reference to two nucleotide sequences, is similar to the concept of percent identity but refers to the percentage of nucleotides of a query sequence that optimally base-pair or hybridize to nucleotides of a subject sequence when the query and subject sequences are linearly arranged and optimally base paired without secondary folding structures, such as loops, stems or hairpins. Such a percent complementarity may be between two DNA strands, two RNA strands, or a DNA strand and a RNA strand. The “percent complementarity” is calculated by (i) optimally base-pairing or hybridizing the two nucleotide sequences in a linear and fully extended arrangement (i.e., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences may be determined based on the known pairings of nucleotide bases, such as G-C, A-T, and A-U, through hydrogen bonding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present disclosure, when two sequences (query and subject) are optimally base-paired (with allowance for mismatches or non-basepaired nucleotides but without folding or secondary structures), the “percent complementarity” for the query sequence is equal to the number of base-paired positions between the two sequences divided by the total number of positions in the query sequence over its length (or by the number of positions in the query sequence over a comparison window), which is then multiplied by 100%.

The term “operably linked” refers to a functional linkage between a promoter or other regulatory element and an associated transcribable DNA sequence or coding sequence of a gene (or transgene), such that the promoter, etc., operates or functions to initiate, assist, affect, cause, and/or promote the transcription and expression of the associated transcribable DNA sequence or coding sequence, at least in certain cell(s), tissue(s), developmental stage(s), and/or condition(s).

The term “plant-expressible promoter” refers to a promoter that can initiate, assist, affect, cause, and/or promote the transcription and expression of its associated transcribable DNA sequence, coding sequence or gene in a plant cell or tissue.

The term “heterologous” in reference to a promoter or other regulatory sequence in relation to an associated polynucleotide sequence (e.g., a transcribable DNA sequence or coding sequence or gene) is a promoter or regulatory sequence that is not operably linked to such associated polynucleotide sequence in nature—e.g., the promoter or regulatory sequence has a different origin relative to the associated polynucleotide sequence and/or the promoter or regulatory sequence is not naturally occurring in a plant species to be transformed with the promoter or regulatory sequence.

The term “recombinant” in reference to a polynucleotide (DNA or RNA) molecule, protein, construct, vector, etc., refers to a polynucleotide or protein molecule or sequence that is man-made and not normally found in nature, and/or is present in a context in which it is not normally found in nature, including a polynucleotide (DNA or RNA) molecule, protein, construct, etc., comprising a combination of two or more polynucleotide or protein sequences that would not naturally occur together in the same manner without human intervention, such as a polynucleotide molecule, protein, construct, etc., comprising at least two polynucleotide or protein sequences that are operably linked but heterologous with respect to each other. For example, the term “recombinant” can refer to any combination of two or more DNA or protein sequences in the same molecule (e.g., a plasmid, construct, vector, chromosome, protein, etc.) where such a combination is man-made and not normally found in nature. As used in this definition, the phrase “not normally found in nature” means not found in nature without human introduction. A recombinant polynucleotide or protein molecule, construct, etc., may comprise polynucleotide or protein sequence(s) that is/are (i) separated from other polynucleotide or protein sequence(s) that exist in proximity to each other in nature, and/or (ii) adjacent to (or contiguous with) other polynucleotide or protein sequence(s) that are not naturally in proximity with each other. Such a recombinant polynucleotide molecule, protein, construct, etc., may also refer to a polynucleotide or protein molecule or sequence that has been genetically engineered and/or constructed outside of a cell. For example, a recombinant DNA molecule may comprise any engineered or man-made plasmid, vector, etc., and may include a linear or circular DNA molecule. Such plasmids, vectors, etc., may contain various maintenance elements including a prokaryotic origin of replication and selectable marker, as well as one or more transgenes or expression cassettes perhaps in addition to a plant selectable marker gene, etc.

As used herein, the term “isolated” refers to at least partially separating a molecule from other molecules typically associated with it in its natural state. In one embodiment, the term “isolated” refers to a DNA molecule that is separated from the nucleic acids that normally flank the DNA molecule in its natural state. For example, a DNA molecule encoding a protein that is naturally present in a bacterium would be an isolated DNA molecule if it was not within the DNA of the bacterium from which the DNA molecule encoding the protein is naturally found. Thus, a DNA molecule fused to or operably linked to one or more other DNA molecule(s) with which it would not be associated in nature, for example as the result of recombinant DNA or plant transformation techniques, is considered isolated herein. Such molecules are considered isolated even when integrated into the chromosome of a host cell or present in a nucleic acid solution with other DNA molecules.

As used herein, an “encoding region” or “coding region” refers to a portion of a polynucleotide that encodes a functional unit or molecule (e.g., a mRNA and/or protein).

As used herein, “modified” in the context of a plant, plant seed, plant part, plant cell, and/or plant genome, refers to a plant, plant seed, plant part, plant cell, and/or plant genome comprising an engineered change in the expression level and/or coding sequence of one or more GA oxidase gene(s) relative to a wild-type or control plant, plant seed, plant part, plant cell, and/or plant genome, such as via a transgenic event comprising a coding or transcribable DNA sequence encoding one or more GA2 oxidase mRNA(s) and protein(s), which may be operably linked to a plant-expressible promoter. For clarity, therefore, a modified plant, plant seed, plant part, plant cell, and/or plant genome includes a transgenic plant, plant seed, plant part, plant cell, and/or plant genome having a modified or ectopic expression, expression level and/or expression pattern of one or more GA2 oxidase gene(s) or coding sequence(s) relative to a wild-type or control plant, plant seed, plant part, plant cell, and/or plant genome. Modified plants, plant parts, seeds, etc., may have been subjected to genetic transformation (e.g., without being limiting, via methods of Agrobacterium transformation or microprojectile bombardment), site-directed integration (e.g., without being limiting, via methods using site-specific nucleases), or a combination thereof. Such “modified” plants, plant seeds, plant parts, and plant cells include plants, plant seeds, plant parts, and plant cells that are offspring or derived from “modified” plants, plant seeds, plant parts, and plant cells that retain the molecular change (e.g., transgene) expressing one or more GA2 oxidase genes or coding sequences. A modified seed provided herein may give rise to a modified plant provided herein. A modified plant, plant seed, plant part, plant cell, or plant genome provided herein may comprise a recombinant DNA construct or vector as provided herein. A “modified plant product” may be any product made from a modified plant, plant part, plant cell, or plant chromosome provided herein, or any portion or component thereof.

As used herein, the term “control plant” (or likewise a “control” plant seed, plant part, plant cell and/or plant genome) refers to a plant (or plant seed, plant part, plant cell and/or plant genome) that is used for comparison to a modified plant (or modified plant seed, plant part, plant cell and/or plant genome) and has the same or similar genetic background (e.g., same parental lines, hybrid cross, inbred line, testers, etc.) as the modified plant (or plant seed, plant part, plant cell and/or plant genome), except for the transgene(s) of the modified plant encoding one or more GA2 oxidases. For example, a control plant may be an inbred line that is the same as the inbred line used to make the modified plant, or a control plant may be the product of the same hybrid cross of inbred parental lines as the modified plant, except for the absence in the control plant of any transgenic event(s) expressing one or more GA2 oxidase genes or coding sequences. For purposes of comparison to a modified plant, plant seed, plant part, plant cell and/or plant genome, a “wild-type plant” (or likewise a “wild-type” plant seed, plant part, plant cell and/or plant genome) refers to a non-transgenic and non-genome edited control plant, plant seed, plant part, plant cell and/or plant genome. As used herein, a “control” plant, plant seed, plant part, plant cell and/or plant genome may also be a plant, plant seed, plant part, plant cell and/or plant genome having a similar (but not the same or identical) genetic background to a modified plant, plant seed, plant part, plant cell and/or plant genome, if deemed sufficiently similar for comparison of the characteristics or traits to be analyzed.

As used herein, a “target site” for genome editing refers to the location of a polynucleotide sequence within a plant genome that is bound and cleaved by a site-specific nuclease introducing a double stranded break (or single-stranded nick) into the nucleic acid backbone of the polynucleotide sequence and/or its complementary DNA strand. A target site may comprise at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 29, or at least 30 consecutive nucleotides. A “target site” for a RNA-guided nuclease may comprise the sequence of either complementary strand of a double-stranded nucleic acid (DNA) molecule or chromosome at the target site. A site-specific nuclease may bind to a target site, such as via a non-coding guide RNA (e.g., without being limiting, a CRISPR RNA (crRNA) or a single-guide RNA (sgRNA) as described further below). A noncoding guide RNA provided herein may be complementary to a target site (e.g., complementary to either strand of a double-stranded nucleic acid molecule or chromosome at the target site). It will be appreciated that perfect identity or complementarity may not be required for a noncoding guide RNA to bind or hybridize to a target site. For example, at least 1, at least 2, at least 3, at least 4, at least 5, at least 6, at least 7, or at least 8 mismatches (or more) between a target site and a non-coding RNA may be tolerated. A “target site” also refers to the location of a polynucleotide sequence within a plant genome that is bound and cleaved by another site-specific nuclease that may not be guided by a non-coding RNA molecule, such as a meganuclease, zinc finger nuclease (ZFN), or a transcription activator-like effector nuclease (TALEN), to introduce a double stranded break (or single-stranded nick) into the polynucleotide sequence and/or its complementary DNA strand. As used herein, a “target region” or a “targeted region” refers to a polynucleotide sequence or region that is flanked by two or more target sites. Without being limiting, in some embodiments a target region may be subjected to a mutation, deletion, insertion or inversion. As used herein, “flanked” when used to describe a target region of a polynucleotide sequence or molecule, refers to two or more target sites of the polynucleotide sequence or molecule surrounding the target region, with one target site on each side of the target region.

As used herein, a “donor molecule”, “donor template”, or “donor template molecule” (collectively a “donor template”), which may be a recombinant DNA donor template, is defined as a nucleic acid molecule having a nucleic acid template or insertion sequence for site-directed, targeted insertion or recombination into the genome of a plant cell via repair of a nick or double-stranded DNA break in the genome of a plant cell. For example, a “donor template” may be used for site-directed integration of a transgene or construct into a target site within the genome of a plant. A targeted genome editing technique provided herein may comprise the use of one or more, two or more, three or more, four or more, or five or more donor molecules or templates. A “donor template” may be a single-stranded or double-stranded DNA or RNA molecule or plasmid. An “insertion sequence” of a donor template is a sequence designed for targeted insertion into the genome of a plant cell, which may be of any suitable length, such as to include a construct and/or coding or transcribable DNA sequence encoding one or more GA2 oxidase(s). A donor template may also have at least one homology sequence or homology arm, such as two homology arms, to direct the integration of a mutation or insertion sequence into a target site within the genome of a plant via homologous recombination, wherein the homology sequence or homology arm(s) are identical or complementary, or have a percent identity or percent complementarity, to a sequence at or near the target site within the genome of the plant. When a donor template comprises homology arm(s) and an insertion sequence, the homology arm(s) will flank or surround the insertion sequence of the donor template.

An insertion sequence of a donor template may comprise one or more genes or sequences that each encode a transcribed mRNA sequence and/or a translated protein sequence. A transcribed sequence or gene of a donor template may encode a mRNA and protein of a GA2 oxidase gene. The donor template may be linear or circular and may be single-stranded or double-stranded. A donor template may be delivered to the cell as a naked nucleic acid (e.g., via particle bombardment), as a complex with one or more delivery agents (e.g., liposomes, proteins, poloxamers, T-strand encapsulated with proteins, etc.), or contained in a bacterial or viral delivery vehicle, such as, for example, Agrobacterium tumefaciens or a geminivirus, respectively. An insertion sequence of a donor template provided herein may comprise a coding or transcribable DNA sequence(s) that may be transcribed into a mRNA molecule(s), which may encode a GA2 oxidase protein(s).

A donor template provided herein may comprise at least one, at least two, at least three, at least four, at least five, at least six, at least seven, at least eight, at least nine, or at least ten transgenes and/or coding or transcribable DNA sequences. Without being limiting, a gene or transcribable DNA sequence of a donor template may also include, for example, an insecticidal resistance gene, an herbicide tolerance gene, a nitrogen use efficiency gene, a water use efficiency gene, a nutritional quality gene, a yield improvement gene, a disease resistance gene, a DNA binding gene, a selectable marker gene, an RNAi or suppression construct, a site-specific genome modification enzyme gene, a single guide RNA of a CRISPR/Cas9 system, a geminivirus-based expression cassette, or a plant viral expression vector system. A donor template may comprise a promoter, such as a tissue-specific or tissue-preferred promoter, a constitutive promoter, or an inducible promoter. A donor template may comprise a leader, enhancer, promoter, transcriptional start site, 5′-UTR, one or more exon(s), one or more intron(s), transcriptional termination site, region or sequence, 3′-UTR, and/or polyadenylation signal. The leader, enhancer, and/or promoter may be operably linked to a coding or transcribable DNA sequence encoding a GA2 oxidase mRNA and/or protein.

As used herein, a “vascular promoter” refers to a plant-expressible promoter that drives, causes or initiates expression of a transcribable DNA sequence or transgene operably linked to such promoter in one or more vascular tissue(s) of the plant, even if the promoter is also expressed in other non-vascular plant cell(s) or tissue(s). Such vascular tissue(s) may comprise one or more of the phloem, vascular parenchymal, and/or bundle sheath cell(s) or tissue(s) of the plant. A “vascular promoter” is distinguished from a constitutive promoter in that it has a regulated and relatively more limited pattern of expression that includes one or more vascular tissue(s) of the plant. A vascular promoter includes both vascular-specific promoters and vascular-preferred promoters.

As used herein, a “leaf promoter” refers to a plant-expressible promoter that drives, causes or initiates expression of a transcribable DNA sequence or transgene operably linked to such promoter in one or more leaf tissue(s) of the plant, even if the promoter is also expressed in other non-leaf plant cell(s) or tissue(s). A leaf promoter includes both leaf-specific promoters and leaf-preferred promoters. A “leaf promoter” is distinguished from a vascular promoter in that it is expressed more predominantly or exclusively in leaf tissue(s) of the plant relative to other plant tissues, whereas a vascular promoter is expressed in vascular tissue(s) more generally including vascular tissue(s) outside of the leaf, such as the vascular tissue(s) of the stem, or stem and leaves, of the plant.

As used herein, a “plant-expressible promoter” refers to a promoter that drives, causes or initiates expression of a transcribable DNA sequence or transgene operably linked to such promoter in one or more plant cells or tissues, such as one or more cells or tissues of a corn plant.

### DESCRIPTION

Most grain producing grasses, such as wheat, rice and Sorghum, produce both male and female structures within each floret of the panicle (i.e., they have a single reproductive structure). However, corn or maize is unique among the grain-producing grasses in that it forms separate male (tassel) and female (ear) inflorescences. Corn produces completely sexually dimorphic reproductive structures by selective abortion of male organs (anthers) in florets of the ear, and female organs (ovules) in the florets of the tassel within early stages of development. Precisely regulated gibberellin synthesis and signaling is critical to regulation of this selective abortion process, with the female reproductive ear being most sensitive to disruptions in the GA pathway. Indeed, the “anther ear” phenotype is the most common reproductive phenotype in GA corn mutants.

In contrast to corn, mutations in the gibberellin synthesis or signaling pathways that led to the “Green Revolution” in wheat, rice and Sorghum had little impact on their reproductive structures because these crop species do not undergo the selective abortion process of the grain bearing panicle during development, and thus are not sensitive to disruptions in GA levels. The same mutations have not historically been utilized in corn because disruption of the GA synthesis and signaling pathway has repeatedly led to dramatic distortion and masculinization of the ear (“anther ear”) and sterility (disrupted anther and microspore development) in the tassel, in addition to extreme dwarfing in some cases. See, e.g., Chen, Y. et al., “The Maize DWARF1 Encodes a Gibberellin 3-Oxidase and Is Dual Localized to the Nucleus and Cytosol,” Plant Physiology 166: 2028-2039 (2014). These earlier GA mutant phenotypes (off-types) in corn led to significant reductions in kernel production and a reduction in yield. Furthermore, production of anthers within the ear increases the likelihood of fungal or insect infections, which reduces the quality of the grain that is produced on those mutant ears. Forward breeding to develop semi-dwarf lines of corn has not been successful, and the reproductive off-types (as well as the extreme dwarfing) of GA mutants have been challenging to overcome.

Despite these prior difficulties in achieving higher grain yields in corn through manipulation of the GA pathway, the present disclosure provides a way to manipulate GA levels in corn plants in a manner that reduces overall plant height and stem internode length and increases resistance to lodging, without causing the reproductive off-types previously associated with mutations of the GA pathway in corn. These short stature or semi-dwarf corn plants may also have one or more additional traits, such as increased stem diameter, reduced green snap, deeper roots, increased leaf area, earlier canopy closure, higher stomatal conductance, lower ear height, increased foliar water content, improved drought tolerance, increased nitrogen use efficiency, increased water use efficiency, reduced anthocyanin content and area in leaves under normal or nitrogen or water limiting stress conditions, increased yield, increased harvest index, increased ear weight, increased kernel number, and/or increased kernel weight.

Without being bound by theory, it is proposed that ectopic expression or overexpression of GA2 oxidase transgene(s) may be effective in achieving a short stature, semi-dwarf phenotype with increased resistance to lodging, but without reproductive off-types in the ear. It is further proposed, without being limited by theory, that restricting the expression of GA2 oxidase gene(s) to certain active GA-producing tissues, such as the vascular and/or leaf tissues of the plant, may be sufficient to produce a short-stature plant with increased lodging resistance, but without significant off-types in reproductive tissues. Expression of a GA2 oxidase transgene in a tissue-specific or tissue-preferred manner may be sufficient and effective at producing plants with the short stature phenotype, while avoiding potential off-types in reproductive tissues that were previously observed with GA mutants in corn (e.g., by avoiding or limiting the expression of the GA2 oxidase gene(s) in those reproductive tissues). For example, the GA2 oxidase transgene(s) may be expressed using a vascular promoter, such as a rice tungro bacilliform virus (RTBV) promoter, that drives expression in vascular tissues of plants. The expression pattern of the RTBV promoter is enriched in vascular tissues of corn plants relative to non-vascular tissues, which is sufficient to produce a semi-dwarf phenotype in corn plants when operably linked to a transcribable DNA sequence encoding a GA2 oxidase gene(s). Lowering of active GA levels in tissue(s) of a corn plant that produce active GAs may reduce plant height and increase lodging resistance, and off-types may be avoided in those plants if active GA levels are not also significantly impacted or lowered in reproductive tissues, such as the developing female organ or ear of the plant. If active GA levels could be reduced in the stalk, stem, or internode(s) of corn plants without significantly affecting GA levels in reproductive tissues (e.g., the female or male reproductive organs or inflorescences), then corn plants having reduced plant height and increased lodging resistance could be created without off-types in the reproductive tissues of the plant.

Thus, recombinant DNA constructs and transgenic plants are provided herein comprising a transcribable DNA sequence encoding a GA2 oxidase mRNA and protein operably linked to a plant expressible promoter, which may be a tissue-specific or tissue-preferred promoter. Such a tissue-specific or tissue-preferred promoter may drive expression of its associated GA2 oxidase coding sequence in one or more active GA-producing tissue(s) of the plant to reduce the level of active GAs produced in those tissue(s). Such a tissue-specific or tissue-preferred promoter may drive expression of its associated GA2 oxidase transgene or coding sequence during one or more vegetative stage(s) of development. Such a tissue-specific or tissue-preferred promoter may also have little or no expression in one or more cell(s) or tissue(s) of the developing female organ or ear of the plant to avoid the possibility of off-types in those reproductive tissues. According to some embodiments, the tissue-specific or tissue-preferred promoter is a vascular promoter, such as the RTBV promoter. The sequence of the RTBV promoter is provided herein as SEQ ID NO: 333, and a truncated version of the RTBV promoter is further provided herein as SEQ ID NO: 334.

Active or bioactive gibberellic acids (i.e., “active gibberellins” or “active GAs”) are known in the art for a given plant species, as distinguished from inactive GAs. For example, active GAs in corn and higher plants include the following: GA1, GA3, GA4, and GA7. Thus, an “active GA-producing tissue” is a plant tissue that produces one or more active GAs.

In addition to suppressing GA20 oxidase genes in active GA-producing tissues of the plant with a vascular tissue promoter, it is further proposed that GA2 oxidase transgenes may also be expressed with various constitutive promoters to cause the short, semi-dwarf stature phenotypes in corn, without any visible off-types in the ear. Thus, it is further proposed that expression of one or more GA2 oxidase transgenes could be carried out using a constitutive promoter to create a short stature, lodging-resistant corn plant without any significant or observable reproductive off-types in the plant.

Without being limited by theory, it is proposed that short stature, semi-dwarf phenotypes in corn plants may result from a sufficient level of expression of a GA2 oxidase transgene(s) in active GA-producing tissue(s) of the plant, and restricting the pattern of expression to avoid reproductive ear tissues may not be necessary to avoid reproductive off-types in the developing ear. It is proposed that the semi-dwarf phenotype with GA2 oxidase overexpression can be the result of shortening the stem internodes of the plant. Without being bound by theory, it is proposed that expression of GA2 oxidase transgene(s) in tissue(s) and/or cell(s) of the plant where active GAs are produced, and not necessarily in stem or internode tissue(s), may be sufficient to produce semi-dwarf plants, even though the short stature trait is due to shortening of the stem internodes. Given that GAs can migrate through the vasculature of the plant, it is proposed that manipulating GA oxidase genes in plant tissue(s) where active GAs are produced may result in a short stature, semi-dwarf plant, even though this may be largely achieved by reducing the level of active GAs produced in non-stem tissues (i.e., away from the site of action in the stem where reduced internode elongation leads to the semi-dwarf phenotype). However, without being bound by theory, expression of a GA2 oxidase transgene at low levels, and/or in a limited number of plant tissues, may be insufficient to cause a significant short stature, semi-dwarf phenotype.

According to embodiments of the present disclosure, modified corn plants are provided that have at least one beneficial agronomic trait and at least one female reproductive organ or ear that is substantially or completely free of off-types. The beneficial agronomic trait may include, for example, shorter plant height, shorter internode length in one or more internode(s), larger (thicker) stem or stalk diameter, increased lodging resistance, improved drought tolerance, increased nitrogen use efficiency, increased water use efficiency, deeper roots, larger leaf area, earlier canopy closure, and/or increased harvestable yield. Off-types may include male (tassel or anther) sterility, reduced kernel or seed number, and/or the presence of one or more masculinized or male (or male-like) reproductive structures in the female organ or ear (e.g., anther ear) of the plant. A modified corn plant is provided herein that lacks significant off-types in the reproductive tissues of the plant. Such a modified corn plant may have a female reproductive organ or ear that appears normal relative to a control or wild-type plant. Indeed, modified corn plants are provided that comprise at least one reproductive organ or ear that does not have or exhibit, or is substantially or completely free of, off-types including male sterility, reduced kernel or seed number, and/or masculinized structure(s) in one or more female organs or ears. As used herein, a female organ or ear of a plant, such as corn, is “substantially free” of male reproductive structures if male reproductive structures are absent or nearly absent in the female organ or ear of the plant based on visual inspection of the female organ or ear at later reproductive stages. A female organ or ear of a plant, such as corn, is “completely free” of mature male reproductive structures if male reproductive structures are absent or not observed or observable in the female organ or ear of the plant, such as a corn plant, by visual inspection of the female organ or ear at later reproductive stages. A female organ or ear of a plant, such as corn, without significant off-types and substantially free of male reproductive structures in the ear may have a number of kernels or seeds per female organ or ear of the plant that is at least 90%, at least 95%, at least 96%, at least 97%, at least 98%, at least 99%, at least 99.5%, at least 99.6%, at least 99.7%, at least 99.8%, or at least 99.9% of the number of kernels or seeds per female organ or ear of a wild-type or control plant. Likewise, a female organ or ear of a plant, such as corn, without significant off-types and substantially free of male reproductive structures in the ear may have an average kernel or seed weight per female organ or ear of the plant that is at least 90%, at least 95%, at least 96%, at least 97%, at least 98%, at least 99%, at least 99.5%, at least 99.6%, at least 99.7%, at least 99.8%, or at least 99.9% of the average kernel or seed weight per female organ or ear of a wild-type or control plant. A female organ or ear of a plant, such as corn, that is completely free of mature male reproductive structures may have a number of kernels or seeds per female organ or ear of the plant that is about the same as a wild-type or control plant. In other words, the reproductive development of the female organ or ear of the plant may be normal or substantially normal. However, the number of seeds or kernels per female organ or ear may depend on other factors that affect resource utilization and development of the plant. Indeed, the number of kernels or seeds per female organ or ear of the plant, and/or the kernel or seed weight per female organ or ear of the plant, may be about the same or greater than a wild-type or control plant.

The plant hormone gibberellin plays an important role in a number of plant developmental processes including germination, cell elongation, flowering, embryogenesis and seed development. Certain biosynthetic enzymes (e.g., GA20 oxidase and GA3 oxidase) and catabolic enzymes (e.g., GA2 oxidase) in the GA pathway are critical to affecting active GA levels in plant tissues. While the biosynthetic enzymes can increase the level of active GAs, the catabolic enzymes can reduce the level(s) of active GAs in plants or plant cells. Thus, it is proposed that overexpression or ectopic expression of a GA2 oxidase transgene in a constitutive or tissue-specific or tissue-preferred manner may produce corn plants having a short stature phenotype and increased lodging resistance, with possible increased yield, but without off-types in the ear. Thus, according to some embodiments, constructs and transgenes are provided comprising a transcribable DNA sequence encoding a GA2 oxidase protein operably linked to a constitutive or tissue-specific or tissue-preferred promoter, such as a vascular or leaf promoter. According to some embodiments, the tissue-specific or tissue-preferred promoter is a vascular promoter, such as the RTBV promoter. However, other types of tissue-specific or tissue preferred promoters may potentially be used for GA2 oxidase expression in active GA-producing tissues of a corn plant to produce a semi-dwarf phenotype without significant off-types.

According to some embodiments, a modified or transgenic plant is provided having a GA2 oxidase gene expression level that is increased in at least one plant tissue by at least 5%, at least 10%, at least 20%, at least 25%, at least 30%, at least 40%, at least 50%, at least 60%, at least 70%, at least 75%, at least 80%, at least 90%, or 100%, as compared to a control plant. According to some embodiments, a modified or transgenic plant is provided having a GA2 oxidase gene expression level that is increased in at least one plant tissue by 5%-20%, 5%25%, 5%-30%, 5%-40%, 5%-50%, 5%-60%, 5%-70%, 5%-75%, 5%-80%, 5%-90%, 5%100%, 75%-100%, 50%-100%, 50%-90%, 50%-75%, 25%-75%, 30%-80%, or 10%-75%, as compared to a control plant. According to these embodiments, the at least one tissue of a modified or transgenic plant having an increased expression level of a GA2 oxidase gene(s) includes one or more active GA producing tissue(s) of the plant, such as the vascular and/or leaf tissue(s) of the plant, during one or more vegetative stage(s) of development.

As understood in the art, the term “promoter” may generally refer to a DNA sequence that contains an RNA polymerase binding site, transcription start site, and/or TATA box and assists or promotes the transcription and expression of an associated transcribable polynucleotide sequence and/or gene (or transgene). A promoter may be synthetic or artificial and/or engineered, varied or derived from a known or naturally occurring promoter sequence. A promoter may be a chimeric promoter comprising a combination of two or more heterologous sequences. A promoter of the present invention may thus include variants of promoter sequences that are similar in composition, but not identical to, other promoter sequence(s) known or provided herein. A promoter may be classified according to a variety of criteria relating to the pattern of expression of an associated coding or transcribable sequence or gene (including a transgene) operably linked to the promoter, such as constitutive, developmental, tissue-specific, inducible, etc. Promoters that drive expression in all or nearly all tissues of the plant are referred to as “constitutive” promoters. However, the expression level with a “constitutive promoter” is not necessarily uniform across different tissue types and cells. Promoters that drive expression during certain periods or stages of development are referred to as “developmental” promoters. Promoters that drive enhanced expression in certain tissues of the plant relative to other plant tissues are referred to as “tissue-enhanced” or “tissue-preferred” promoters. Thus, a “tissue-preferred” promoter causes relatively higher or preferential or predominant expression in a specific tissue(s) of the plant, but with lower levels of expression in other tissue(s) of the plant. Promoters that express within a specific tissue(s) of the plant, with little or no expression in other plant tissues, are referred to as “tissue-specific” promoters. A tissue-specific or tissue-preferred promoter may also be defined in terms of the specific or preferred tissue(s) in which it drives expression of its associated transcribable DNA sequence or suppression element. For example, a promoter that causes specific expression in vascular tissues may be referred to as a “vascular-specific promoter”, whereas a promoter that causes preferential or predominant expression in vascular tissues may be referred to as a “vascular-preferred promoter”. Likewise, a promoter that causes specific expression in leaf tissues may be referred to as a “leaf-specific promoter”, whereas a promoter that causes preferential or predominant expression in leaf tissues may be referred to as a “leaf-preferred promoter”. An “inducible” promoter is a promoter that initiates transcription in response to an environmental stimulus such as cold, drought or light, or other stimuli, such as wounding or chemical application. A promoter may also be classified in terms of its origin, such as being heterologous, homologous, chimeric, synthetic, etc. A “heterologous” promoter is a promoter sequence having a different origin relative to its associated transcribable sequence, coding sequence, or gene (or transgene), and/or not naturally occurring in the plant species to be transformed, as defined above.

In some embodiments, transgenic expression of a GA2 oxidase transgene is constitutive or tissue-specific (e.g., only in leaf and/or vascular tissue). For example, expression of a GA2 oxidase transgene may be vascular or leaf tissue specific or preferred. In other embodiments, expression of a GA2 oxidase transgene is constitutive and not tissue-specific. According to some embodiments, expression of a GA2 oxidase transgene is increased in one or more tissue types (e.g., in leaf and/or vascular tissue(s)) of a modified or transgenic plant as compared to the same tissue(s) of a control plant.

According to embodiments of the present disclosure, a recombinant DNA molecule, construct or vector is provided comprising an expression cassette comprising a GA2 oxidase coding sequence or transcribable DNA sequence that is operably linked to a plant-expressible constitutive or tissue-specific or tissue-preferred promoter. The expression cassette may comprise a transcribable DNA sequence having a percent identity to all or part of a GA2 oxidase gene or coding sequence. A transgene having a coding sequence with a lower percent identity to all or part of a GA2 oxidase gene may encode a protein having or retaining a GA catabolic activity in a corn plant or plant cell similar to GA2 oxidase genes in general.

A single GA2 oxidase transgene or expression cassette may be present in a construct, molecule or vector, or multiple GA2 oxidase transgenes or expression cassettes may be arranged serially in tandem or arranged in tandem segments or repeats, in a construct, molecule or vector, which may also be interrupted by one or more spacer sequence(s). The sequence of each transgene or expression cassette may encode a GA2 oxidase mRNA and protein. A transcribable DNA sequence or coding sequence of a GA2 oxidase transgene may encode a protein having at least 70%, at least 75%, at least 80%, at least 85%, at least 90%, at least 95%, at least 96%, at least 97%, at least 98%, at least 99%, at least 99.5% or 100% identity to all or part of a GA2 oxidase gene sequence.

According to embodiments of the present disclosure, a recombinant DNA molecule, construct or vector is provided comprising a transcribable DNA sequence encoding a GA2 oxidase. According to some embodiments, a recombinant DNA molecule, vector or construct is provided comprising a transcribable DNA sequence encoding a GA2 oxidase mRNA and protein in a plant cell, and wherein the transcribable DNA sequence is operably linked to a plant-expressible promoter, such as a constitutive or tissue-specific or tissue-preferred promoter. According to embodiments of the present disclosure, suitable tissue-specific or tissue preferred promoters for expression of a GA2 oxidase may include those promoters that drive or cause expression of its associated suppression element or sequence at least in the vascular and/or leaf tissue(s) of a corn plant. Expression of the GA2 oxidase with a tissue-specific or tissue-preferred promoter may also occur in other tissues of the corn plant outside of the vascular and leaf tissues, but active GA levels in the developing reproductive tissues of the plant (particularly in the female reproductive organ or ear) are preferably not significantly reduced or impacted (relative to wild type or control plants), such that development of the female organ or ear may proceed normally in the transgenic plant without off-types in the ear and a loss in yield potential. According to many embodiments, the plant-expressible promoter may preferably drive expression constitutively or in at least a portion of the vascular and/or leaf tissues of the plant. However, some tissue-specific and tissue-preferred promoters driving expression of a GA2 oxidase transgene in a plant may not produce a significant short stature or anti-lodging phenotypes due to the spatial-temporal pattern of expression of the promoter during plant development, and/or the amount or strength of expression of the promoter being too low or weak. A sufficient level of expression of a transcribable DNA sequence encoding a GA2 oxidase may be necessary to produce a short stature, semi-dwarf phenotype that resists lodging, since lower levels of expression may be insufficient to lower active GA levels in the plant to a sufficient extent to cause a significant phenotype. Thus, tissue-specific and tissue-preferred promoters that drive, etc., a moderate or strong level of expression of their associated transcribable DNA sequence in active GA-producing tissue(s) of a plant may be preferred. Furthermore, such tissue-specific and tissue-preferred should drive, etc., expression of their associated transcribable DNA sequence during one or more vegetative stage(s) of plant development when the plant is growing and/or elongating including one or more of the following vegetative stage(s): V_{E}, V1, V2, V3, V4, V5, V6, V7, V8, V9, V10, V11, V12, V13, V14, Vn, V_{T}, such as expression at least during V3-V12, V4-V12, V5-V12, V6-V12, V7-V12, V8-V12, V3-V14, V5-V14, V6-V14, V7-V14, V8-V14, V9-V14, V10-V14, etc., or during any other range of vegetative stages when growth and/or elongation of the plant is occurring.

Any vascular promoters known in the art may potentially be used as the tissue-specific or tissue-preferred promoter. Examples of vascular promoters include the RTBV promoter (see, e.g., SEQ ID NO: 333), a known sucrose synthase gene promoter, such as a corn sucrose synthase-1 (Sus1 or Sh1) promoter (see, e.g., SEQ ID NO: 335), a corn Sh1 gene paralog promoter, a barley sucrose synthase promoter (Ss1) promoter, a rice sucrose synthase-1 (RSs1) promoter (see, e.g., SEQ ID NO: 336), or a rice sucrose synthase-2 (RSs2) promoter (see, e.g., SEQ ID NO: 337), a known sucrose transporter gene promoter, such as a rice sucrose transporter promoter (SUT1) (see, e.g., SEQ ID NO: 338), or various known viral promoters, such as a Commelina yellow mottle virus (CoYMV) promoter, a wheat dwarf geminivirus (WDV) large intergenic region (LIR) promoter, a maize streak geminivirus (MSV) coat protein (CP) promoter, or a rice yellow stripe 1 (YS1)-like or OsYSL2 promoter (SEQ ID NO: 339), and any functional sequence portion or truncation of any of the foregoing promoters with a similar pattern of expression, such as a truncated RTBV promoter (see, e.g., SEQ ID NO: 334). Any other vascular promoters known in the art may also be used, including promoter sequences from related genes (e.g., sucrose synthase, sucrose transporter, and viral gene promoter sequences) from the same or different plant species, microbe or virus that have a similar pattern of expression. Further provided are promoter sequences with a high degree of homology to any of the foregoing. For example, a vascular promoter may comprise a DNA sequence that is at least at least 70%, at least 80%, at least 85%, at least 90%, at least 95%, at least 96%, at least 97%, at least 98%, at least 99%, at least 99.5% or 100% identical to one or more of SEQ ID NOs: 333, 334, 335, 336, 337, 338, and 339, any functional sequence portion or truncation thereof, and/or any sequence complementary to any of the foregoing sequences. Examples of vascular promoters may further include other known, engineered and/or later-identified promoter sequences shown to have a pattern of expression in vascular tissue(s) of a corn plant.

Any leaf promoters known in the art may potentially be used as the tissue-specific or tissue-preferred promoter. Examples of leaf promoters include a corn pyruvate phosphate dikinase or PPDK promoter (see, e.g., SEQ ID NO: 340), a corn fructose 1,6 bisphosphate aldolase or FDA promoter (see, e.g., SEQ ID NO: 341), and a rice Nadh-Gogat promoter (see, e.g., SEQ ID NO: 342), and any functional sequence portion or truncation of any of the foregoing promoters with a similar pattern of expression. Other examples of leaf promoters from monocot plant genes include a ribulose biphosphate carboxylase (RuBisCO) or RuBisCO small subunit (RBCS) promoter, a chlorophyll a/b binding protein gene promoter, a phosphoenolpyruvate carboxylase (PEPC) promoter, and a Myb gene promoter, and any functional sequence portion or truncation of any of these promoters with a similar pattern of expression. Any other leaf promoters known in the art may also be used, including promoter sequences from related genes from the same or different plant species, microbe or virus that have a similar pattern of expression. Further provided are promoter sequences with a high degree of homology to any of the foregoing. For example, a leaf promoter may comprise a DNA sequence that is at least at least 70%, at least 80%, at least 85%, at least 90%, at least 95%, at least 96%, at least 97%, at least 98%, at least 99%, at least 99.5% or 100% identical to one or more of SEQ ID NOs: 340, 341, and 342, any functional sequence portion or truncation thereof, and/or any sequence complementary to any of the foregoing sequences. Examples of leaf promoters may further include other known, engineered and/or later-identified promoter sequences shown to have a pattern of expression in leaf tissue(s) of a corn plant.

Any constitutive promoters known in the art may potentially be used. Examples of constitutive promoters that may be used in corn plants include, for example, various actin gene promoters, such as a rice Actin 1 promoter (see, e.g., U.S. Pat. No. 5,641,876; see also SEQ ID NO: 343 or SEQ ID NO: 344) and a rice Actin 2 promoter (see, e.g., U.S. Pat. No. 6,429,357; see also, e.g., SEQ ID NO: 345 or SEQ ID NO: 346), a CaMV 35S or 19S promoter (see, e.g., U.S. Pat. No. 5,352,605; see also, e.g., SEQ ID NO: 347 for CaMV 35S), a maize ubiquitin promoter (see, e.g., U.S. Pat. No. 5,510,474), a Coix lacryma-jobi polyubiquitin promoter (see, e.g., SEQ ID NO: 348), a rice or maize Gos2 promoter (see, e.g., Pater et al., The Plant Journal, 2(6): 837-44 1992; see also, e.g., SEQ ID NO: 349 for the rice Gos2 promoter), a FMV 35S promoter (see, e.g., U.S. Pat. No. 6,372,211), a dual enhanced CMV promoter (see, e.g., U.S. Pat. No. 5,322,938), a MMV promoter (see, e.g., U.S. Pat. No. 6,420,547; see also, e.g., SEQ ID NO: 350), a PCLSV promoter (see, e.g., U.S. Pat. No. 5,850,019; see also, e.g., SEQ ID NO: 351), an Emu promoter (see, e.g., Last et al., Theor. Appl. Genet. 81:581 (1991); and Mcelroy et al., Mol. Gen. Genet. 231:150 (1991)), a tubulin promoter from maize, rice or other species, a nopaline synthase (nos) promoter, an octopine synthase (ocs) promoter, a mannopine synthase (mas) promoter, or a plant alcohol dehydrogenase (e.g., maize Adh1) promoter, any other promoters including viral promoters known or later-identified in the art to provide constitutive expression in a corn plant, any other constitutive promoters known in the art that may be used in corn plants, and any functional sequence portion or truncation of any of the foregoing promoters.

Any other constitutive promoters known in the art may also be used, including promoter sequences from related genes from the same or different plant species, microbe or virus that have a similar pattern of expression. Further provided are promoter sequences with a high degree of homology to any of the foregoing. For example, a constitutive promoter may comprise a DNA sequence that is at least at least 70%, at least 80%, at least 85%, at least 90%, at least 95%, at least 96%, at least 97%, at least 98%, at least 99%, at least 99.5% or 100% identical to one or more of SEQ ID NOs: 343, 344, 345, 346, 347, 348, 349, 350, and 351, any functional sequence portion or truncation thereof, and/or any sequence complementary to any of the foregoing sequences. Examples of constitutive promoters may further include other known, engineered and/or later-identified promoter sequences shown to have a constitutive pattern of expression in a corn plant. Furthermore, any known or later-identified constitutive promoter may also be used.

According to embodiments of the present disclosure, a recombinant DNA molecule, construct or vector is provided comprising a transcribable DNA sequence encoding a GA2 oxidase protein from a monocot or cereal plant, such as a corn plant, or encoding a GA2 oxidase protein having at least a certain percent homology to a GA2 oxidase protein from a monocot or cereal plant, such as a corn plant. A family of at least thirteen GA2 oxidase genes have been identified in corn (Zea mays) including Zm.GA2 oxidase_1, Zm.GA2 oxidase_2, Zm.GA2 oxidase_3, Zm.GA2 oxidase_4, Zm.GA2 oxidase_5, Zm.GA2 oxidase_6, Zm.GA2 oxidase_7, Zm.GA2 oxidase_8, Zm.GA2 oxidase_9, Zm.GA2 oxidase_10, Zm.GA2 oxidase_11, Zm.GA2 oxidase_12, and Zm.GA2 oxidase_13. The DNA and protein sequences by SEQ ID NO for each of these GA2 oxidase genes are provided in Table 1.

TABLE 1
DNA and protein sequences for GA2 oxidase genes in corn.

| | Coding Sequence | |
|---|---|---|
| GA2 oxidase Gene | (CDS) | Protein |
| Zm.GA2 oxidase_1 | SEQ ID NO: 1 | SEQ ID NO: 2 |
| Zm.GA2 oxidase_2 | SEQ ID NO: 3 | SEQ ID NO: 4 |
| Zm.GA2 oxidase_3 | SEQ ID NO: 5 | SEQ ID NO: 6 |
| Zm.GA2 oxidase_4 | SEQ ID NO: 7 | SEQ ID NO: 8 |
| Zm.GA2 oxidase_5 | SEQ ID NO: 9 | SEQ ID NO: 10 |
| Zm.GA2 oxidase_6 | SEQ ID NO: 11 | SEQ ID NO: 12 |
| Zm.GA2 oxidase_7 | SEQ ID NO: 13 | SEQ ID NO: 14 |
| Zm.GA2 oxidase_8 | SEQ ID NO: 15 | SEQ ID NO: 16 |
| Zm.GA2 oxidase_9 | SEQ ID NO: 17 | SEQ ID NO: 18 |
| Zm.GA2 oxidase_10 | SEQ ID NO: 19 | SEQ ID NO: 20 |
| Zm.GA2 oxidase_11 | SEQ ID NO: 21 | SEQ ID NO: 22 |
| Zm.GA2 oxidase_12 | SEQ ID NO: 23 | SEQ ID NO: 24 |
| Zm.GA2 oxidase_13 | SEQ ID NO: 25 | SEQ ID NO: 26 |

GA2 oxidase genes from other monocot or cereal plant species may also be used, such as rice, barley, wheat and Sorghum. According to embodiments of the present disclosure, a recombinant DNA molecule, construct or vector is provided comprising a transcribable DNA sequence encoding a GA2 oxidase protein from a monocot or cereal plant other than corn, or encoding a GA2 oxidase protein having at least a certain percent homology to a GA2 oxidase protein from a monocot or cereal plant other than corn. A family of at least ten GA2 oxidase genes have been identified in rice (Oryza sativa) plants including Os.GA2 oxidase_1, Os.GA2 oxidase_2, Os.GA2 oxidase_3, Os.GA2 oxidase_4, Os.GA2 oxidase_5, Os.GA2 oxidase_6, Os.GA2 oxidase_7, Os.GA2 oxidase_8, Os.GA2 oxidase_9, and Os.GA2 oxidase_10. The DNA and protein sequences by SEQ ID NO for each of these GA2 oxidase genes are provided in Table 2.

TABLE 2
DNA and protein sequences for GA2 oxidase genes in rice.

| | Coding Sequence | |
|---|---|---|
| GA2 oxidase Gene | (CDS) | Protein |
| Os.GA2 oxidase_1 | SEQ ID NO: 27 | SEQ ID NO: 28 |
| Os.GA2 oxidase_2 | SEQ ID NO: 29 | SEQ ID NO: 30 |
| Os.GA2 oxidase_3 | SEQ ID NO: 31 | SEQ ID NO: 32 |
| Os.GA2 oxidase_4 | SEQ ID NO: 33 | SEQ ID NO: 34 |
| Os.GA2 oxidase_5 | SEQ ID NO: 35 | SEQ ID NO: 36 |
| Os.GA2 oxidase_6 | SEQ ID NO: 37 | SEQ ID NO: 38 |
| Os.GA2 oxidase_7 | SEQ ID NO: 39 | SEQ ID NO: 40 |
| Os.GA2 oxidase_8 | SEQ ID NO: 41 | SEQ ID NO: 42 |
| Os.GA2 oxidase_9 | SEQ ID NO: 43 | SEQ ID NO: 44 |
| Os.GA2 oxidase_10 | SEQ ID NO: 45 | SEQ ID NO: 46 |

A family of at least eight GA2 oxidase genes have been identified in barley (Hordeum vulgare) plants including Hv.GA2 oxidase_1, Hv.GA2 oxidase_2, Hv.GA2 oxidase_3, Hv.GA2 oxidase_4 HvGA2 oxidase_5, Hv.GA2 oxidase_6, Hv.GA2 oxidase_7; and Hv.GA2 oxidase_8. The DNA and protein sequences by SEQ ID NO for each of these GA2 oxidase genes are provided in Table 3.

TABLE 3
DNA and protein sequences for GA2 oxidase genes in barley.

| | Coding Sequence | |
|---|---|---|
| GA2 oxidase Gene | (CDS) | Protein |
| Hv.GA2 oxidase_1 | SEQ ID NO: 47 | SEQ ID NO: 48 |
| Hv.GA2 oxidase_2 | SEQ ID NO: 49 | SEQ ID NO: 50 |
| Hv.GA2 oxidase_3 | SEQ ID NO: 51 | SEQ ID NO: 52 |
| Hv.GA2 oxidase_4 | SEQ ID NO: 53 | SEQ ID NO: 54 |
| Hv.GA2 oxidase_5 | SEQ ID NO: 55 | SEQ ID NO: 56 |
| Hv.GA2 oxidase_6 | SEQ ID NO: 57 | SEQ ID NO: 58 |
| Hv.GA2 oxidase_7 | SEQ ID NO: 59 | SEQ ID NO: 60 |
| Hv.GA2 oxidase_8 | SEQ ID NO: 61 | SEQ ID NO: 62 |

A family of at least sixteen GA2 oxidase genes have been identified in Sorghum (Sorghum bicolor) plants including Hv.GA2 oxidase_1, Sb.GA2 oxidase_2, Sb.GA2 oxidase_3, Sb.GA2 oxidase_4, Sb.GA2 oxidase_5, Sb.GA2 oxidase_6, Sb.GA2 oxidase_7 Sb.GA2 oxidase_8, Sb.GA2 oxidase_9, Sb.GA2 oxidase_10, Sb.GA2 oxidase_11, Sb.GA2 oxidase_12, Sb.GA2 oxidase_13, Sb.GA2 oxidase_14, Sb.GA2 oxidase_15, and Sb.GA2 oxidase_16. The DNA and protein sequences by SEQ ID NO for each of these GA2 oxidase genes are provided in Table 4.

TABLE 4
DNA and protein sequences for GA2 oxidase genes in sorghum.

| | Coding Sequence | |
|---|---|---|
| GA2 oxidase Gene | (CDS) | Protein |
| Sb.GA2 oxidase_1 | SEQ ID NO: 63 | SEQ ID NO: 64 |
| Sb.GA2 oxidase_2 | SEQ ID NO: 65 | SEQ ID NO: 66 |
| Sb.GA2 oxidase_3 | SEQ ID NO: 67 | SEQ ID NO: 68 |
| Sb.GA2 oxidase_4 | SEQ ID NO: 69 | SEQ ID NO: 70 |
| Sb.GA2 oxidase_5 | SEQ ID NO: 71 | SEQ ID NO: 72 |
| Sb.GA2 oxidase_6 | SEQ ID NO: 73 | SEQ ID NO: 74 |
| Sb.GA2 oxidase_7 | SEQ ID NO: 75 | SEQ ID NO: 76 |
| Sb.GA2 oxidase_8 | SEQ ID NO: 77 | SEQ ID NO: 78 |
| Sb.GA2 oxidase_9 | SEQ ID NO: 79 | SEQ ID NO: 80 |
| Sb.GA2 oxidase_10 | SEQ ID NO: 81 | SEQ ID NO: 82 |
| Sb.GA2 oxidase_11 | SEQ ID NO: 83 | SEQ ID NO: 84 |
| Sb.GA2 oxidase_12 | SEQ ID NO: 85 | SEQ ID NO: 86 |
| Sb.GA2 oxidase_13 | SEQ ID NO: 87 | SEQ ID NO: 88 |
| Sb.GA2 oxidase_14 | SEQ ID NO: 89 | SEQ ID NO: 90 |
| Sb.GA2 oxidase_15 | SEQ ID NO: 91 | SEQ ID NO: 92 |
| Sb.GA2 oxidase_16 | SEQ ID NO: 93 | SEQ ID NO: 94 |

A family of at least fifteen GA2 oxidase genes have been identified in wheat (Triticum aestivum) including Ta.GA2 oxidase_1, Ta.GA2 oxidase_2, Ta.GA2 oxidase_3, Ta.GA2 oxidase_4 Ta.GA2 oxidase_5, Ta.GA2 oxidase_6, Ta.GA2 oxidase_7 Ta.GA2 oxidase_8, Ta.GA2 oxidase_9, Ta.GA2 oxidase_10, Ta.GA2 oxidase_11 Ta.GA2 oxidase_12 Ta.GA2 oxidase_13, Ta.GA2 oxidase_14, and Ta.GA2 oxidase_15. The DNA and protein sequences by SEQ ID NO for each of these GA2 oxidase genes from wheat are provided in Table 5.

TABLE 5
DNA and protein sequences for GA2 oxidase genes in wheat.

| | Coding Sequence | |
|---|---|---|
| GA2 oxidase Gene | (CDS) | Protein |
| Ta.GA2 oxidase_1 | SEQ ID NO: 95 | SEQ ID NO: 96 |
| Ta.GA2 oxidase_2 | SEQ ID NO: 97 | SEQ ID NO: 98 |
| Ta.GA2 oxidase_3 | SEQ ID NO: 99 | SEQ ID NO: 100 |
| Ta.GA2 oxidase_4 | SEQ ID NO: 101 | SEQ ID NO: 102 |
| Ta.GA2 oxidase_5 | SEQ ID NO: 103 | SEQ ID NO: 104 |
| Ta.GA2 oxidase_6 | SEQ ID NO: 105 | SEQ ID NO: 106 |
| Ta.GA2 oxidase_7 | SEQ ID NO: 107 | SEQ ID NO: 108 |
| Ta.GA2 oxidase_8 | SEQ ID NO: 109 | SEQ ID NO: 110 |
| Ta.GA2 oxidase_9 | SEQ ID NO: 111 | SEQ ID NO: 112 |
| Ta.GA2 oxidase_10 | SEQ ID NO: 113 | SEQ ID NO: 114 |
| Ta.GA2 oxidase_11 | SEQ ID NO: 115 | SEQ ID NO: 116 |
| Ta.GA2 oxidase_12 | SEQ ID NO: 117 | SEQ ID NO: 118 |
| Ta.GA2 oxidase_13 | SEQ ID NO: 119 | SEQ ID NO: 120 |
| Ta.GA2 oxidase_14 | SEQ ID NO: 121 | SEQ ID NO: 122 |
| Ta.GA2 oxidase_15 | SEQ ID NO: 123 | SEQ ID NO: 124 |

In addition to the corn sequences listed in Table 1, a family of at least eleven GA2 oxidase genes have been identified in another corn (Zea Mays) germplasm line including Zm2.GA2 oxidase_1, Zm2.GA2 oxidase_2 Zm2GA2 oxidase_3, Zm2.GA2 oxidase_4, Zm2.GA2 oxidase_5, Zm2.GA2 oxidase_6, Zm2.GA2 oxidase_7, Zm2.GA2 oxidase_8, Zm2.GA2 oxidase_9, Zm2.GA2 oxidase_10, and Zm2.GA2 oxidase_11. The DNA and protein sequences by SEQ ID NO for each of these GA2 oxidase genes are provided in Table 6.

TABLE 6
Additional DNA and protein sequences
for GA2 oxidase genes in corn.

| GA2 | Coding Sequence | |
|---|---|---|
| oxidase Gene | (CDS) | Protein |
| Zm2.GA2 oxidase_1 | SEQ ID NO: 125 | SEQ ID NO: 126 |
| Zm2.GA2 oxidase_2 | SEQ ID NO: 127 | SEQ ID NO: 128 |
| Zm2.GA2 oxidase_3 | SEQ ID NO: 129 | SEQ ID NO: 130 |
| Zm2.GA2 oxidase_4 | SEQ ID NO: 131 | SEQ ID NO: 132 |
| Zm2.GA2 oxidase_5 | SEQ ID NO: 133 | SEQ ID NO: 134 |
| Zm2.GA2 oxidase_6 | SEQ ID NO: 135 | SEQ ID NO: 136 |
| Zm2.GA2 oxidase_7 | SEQ ID NO: 137 | SEQ ID NO: 138 |
| Zm2.GA2 oxidase_8 | SEQ ID NO: 139 | SEQ ID NO: 140 |
| Zm2.GA2 oxidase_9 | SEQ ID NO: 141 | SEQ ID NO: 142 |
| Zm2.GA2 oxidase_10 | SEQ ID NO: 143 | SEQ ID NO: 144 |
| Zm2.GA2 oxidase_11 | SEQ ID NO: 145 | SEQ ID NO: 146 |

According to embodiments of the present disclosure, a recombinant DNA molecule, construct or vector is provided comprising a transcribable DNA sequence encoding a GA2 oxidase protein from a dicot plant, such as a soybean, cotton, canola, Arabidopsis, moss (Physcomitrella patens), common bean (Phaseolus vulgaris), cottonwood (Populus trichocarpa), barrel clover (Medicago truncatula), pea (Pisum sativum), spinach (Spinacia oleracea) or whorled o (Paris polyphylla) plant, or encoding a GA2 oxidase protein having at least a certain percent homology to a GA2 oxidase protein from a monocot or cereal plant, such as a corn plant.

A family of at least sixteen GA2 oxidase genes have been identified in soybean (Glycine max) including Gm.GA2 oxidase_1, Gm.GA2 oxidase_2, Gm.GA2 oxidase_3, Gm.GA2 oxidase_4, Gm.GA2 oxidase_5, Gm.GA2 oxidase_6, Gm.GA2 oxidase_7, Gm.GA2 oxidase_8, Gm.GA2 oxidase_9, Gm.GA2 oxidase_10, Gm.GA2 oxidase_11, Gm.GA2 oxidase_12, Gm.GA2 oxidase_13, Gm.GA2 oxidase_14 Gm.GA2 oxidase_15, and Gm.GA2 oxidase_16. The DNA and protein sequences by SEQ ID NO for each of these GA2 oxidase genes from soybean are provided in Table 7.

TABLE 7
Additional DNA and protein sequences
for GA2 oxidase genes in soybean.

| GA2 | Coding Sequence | |
|---|---|---|
| oxidase Gene | (CDS) | Protein |
| Gm.GA2 oxidase_1 | SEQ ID NO: 147 | SEQ ID NO: 148 |
| Gm.GA2 oxidase_2 | SEQ ID NO: 149 | SEQ ID NO: 150 |
| Gm.GA2 oxidase_3 | SEQ ID NO: 151 | SEQ ID NO: 152 |
| Gm.GA2 oxidase_4 | SEQ ID NO: 153 | SEQ ID NO: 154 |
| Gm.GA2 oxidase_5 | SEQ ID NO: 155 | SEQ ID NO: 156 |
| Gm.GA2 oxidase_6 | SEQ ID NO: 157 | SEQ ID NO: 158 |
| Gm.GA2 oxidase_7 | SEQ ID NO: 159 | SEQ ID NO: 160 |
| Gm.GA2 oxidase_8 | SEQ ID NO: 161 | SEQ ID NO: 162 |
| Gm.GA2 oxidase_9 | SEQ ID NO: 163 | SEQ ID NO: 164 |
| Gm.GA2 oxidase_10 | SEQ ID NO: 165 | SEQ ID NO: 166 |
| Gm.GA2 oxidase_11 | SEQ ID NO: 167 | SEQ ID NO: 168 |
| Gm.GA2 oxidase_12 | SEQ ID NO: 169 | SEQ ID NO: 170 |
| Gm.GA2 oxidase_13 | SEQ ID NO: 171 | SEQ ID NO: 172 |
| Gm.GA2 oxidase_14 | SEQ ID NO: 173 | SEQ ID NO: 174 |
| Gm.GA2 oxidase_15 | SEQ ID NO: 175 | SEQ ID NO: 176 |
| Gm.GA2 oxidase_16 | SEQ ID NO: 177 | SEQ ID NO: 178 |

A family of at least fifteen related GA2 oxidase genes have been identified in cotton (Gossypium hirsutum)including Gh.GA2 oxidase_1, Gh.GA2 oxidase_2, Gh.GA2 oxidase_3, Gh.GA2 oxidase_4, Gh.GA2 oxidase_5, Gh.GA2 oxidaseQ6, Gh.GA2 oxidase_7, Gh.GA2 oxidase_8, Gh.GA2 oxidase_9, Gh.GA2 oxidase_10, Gh.GA2 oxidase_1, Gh.GA2 oxidase_12 Gh.GA2 oxidase_13, Gh.GA2 oxidase_14, and Gh.GA2 oxidase_15. The DNA and protein sequences by SEQ ID NO for each of these GA2 oxidase genes from cotton are provided in Table 8.

TABLE 8
DNA and protein sequences for GA2 oxidase genes in cotton.

| GA2 | Coding Sequence | |
|---|---|---|
| oxidase Gene | (CDS) | Protein |
| Gh.GA2 oxidase_1 | SEQ ID NO: 179 | SEQ ID NO: 180 |
| Gh.GA2 oxidase_2 | SEQ ID NO: 181 | SEQ ID NO: 182 |
| Gh.GA2 oxidase_3 | SEQ ID NO: 183 | SEQ ID NO: 184 |
| Gh.GA2 oxidase_4 | SEQ ID NO: 185 | SEQ ID NO: 186 |
| Gh.GA2 oxidase_5 | SEQ ID NO: 187 | SEQ ID NO: 188 |
| Gh.GA2 oxidase_6 | SEQ ID NO: 189 | SEQ ID NO: 190 |
| Gh.GA2 oxidase_7 | SEQ ID NO: 191 | SEQ ID NO: 192 |
| Gh.GA2 oxidase_8 | SEQ ID NO: 193 | SEQ ID NO: 194 |
| Gh.GA2 oxidase_9 | SEQ ID NO: 195 | SEQ ID NO: 196 |
| Gh.GA2 oxidase_10 | SEQ ID NO: 197 | SEQ ID NO: 198 |
| Gh.GA2 oxidase_11 | SEQ ID NO: 199 | SEQ ID NO: 200 |
| Gh.GA2 oxidase_12 | SEQ ID NO: 201 | SEQ ID NO: 202 |
| Gh.GA2 oxidase_13 | SEQ ID NO: 203 | SEQ ID NO: 204 |
| Gh.GA2 oxidase_14 | SEQ ID NO: 205 | SEQ ID NO: 206 |
| Gh.GA2 oxidase_15 | SEQ ID NO: 207 | SEQ ID NO: 208 |

A family of at least fifteen GA2 oxidase genes have been identified in canola (Brassica napus) including Bn.GA2 oxidase_1, Bn.GA2 oxidase_2, Bn.GA2 oxidase_3, Bn.GA2 oxidase_4, Bn.GA2 oxidase_5, Bn.GA2 oxidase_6, Bn.GA2 oxidase_7, Bn.GA2 oxidase_8, Bn.GA2 oxidase_9, Bn.GA2 oxidase_10, Bn.GA2 oxidaseQ11, Bn.GA2 oxidase_12, Bn.GA2 oxidase_13, Bn.GA2 oxidase_14, and Bn.GA2 oxidase_15. The DNA and protein sequences by SEQ ID NO for each of these GA2 oxidase genes from canola are provided in Table 9.

TABLE 9
DNA and protein sequences for GA2 oxidase genes in canola.

| GA2 | Coding Sequence | |
|---|---|---|
| oxidase Gene | (CDS) | Protein |
| Bn.GA2 oxidase_1 | SEQ ID NO: 209 | SEQ ID NO: 210 |
| Bn.GA2 oxidase_2 | SEQ ID NO: 211 | SEQ ID NO: 212 |
| Bn.GA2 oxidase_3 | SEQ ID NO: 213 | SEQ ID NO: 214 |
| Bn.GA2 oxidase_4 | SEQ ID NO: 215 | SEQ ID NO: 216 |
| Bn.GA2 oxidase_5 | SEQ ID NO: 217 | SEQ ID NO: 218 |
| Bn.GA2 oxidase_6 | SEQ ID NO: 219 | SEQ ID NO: 220 |
| Bn.GA2 oxidase_7 | SEQ ID NO: 221 | SEQ ID NO: 222 |
| Bn.GA2 oxidase_8 | SEQ ID NO: 223 | SEQ ID NO: 224 |
| Bn.GA2 oxidase_9 | SEQ ID NO: 225 | SEQ ID NO: 226 |
| Bn.GA2 oxidase_10 | SEQ ID NO: 227 | SEQ ID NO: 228 |
| Bn.GA2 oxidase_11 | SEQ ID NO: 229 | SEQ ID NO: 230 |
| Bn.GA2 oxidase_12 | SEQ ID NO: 231 | SEQ ID NO: 232 |
| Bn.GA2 oxidase_13 | SEQ ID NO: 233 | SEQ ID NO: 234 |
| Bn.GA2 oxidase_14 | SEQ ID NO: 235 | SEQ ID NO: 236 |
| Bn.GA2 oxidase_15 | SEQ ID NO: 237 | SEQ ID NO: 238 |

A family of at least seven GA2 oxidase genes have been identified in thale cress (Arabidopsis thaliana) including At.GA2 oxidase_1, At.GA2 oxidase_2, At.GA2 oxidase_3, At.GA2 oxidase_4, At.GA2 oxidase_6, and At.GA2 oxidase_7, and At.GA2 oxidase_8. The DNA and protein sequences by SEQ ID NO for each of these GA2 oxidase genes from Arabidopsis are provided in Table 10.

TABLE 10
DNA and protein sequences for GA2 oxidase genes in thale cress.

| GA2 | Coding Sequence | |
|---|---|---|
| oxidase Gene | (CDS) | Protein |
| At.GA2 oxidase_1 | SEQ ID NO: 239 | SEQ ID NO: 240 |
| At.GA2 oxidase_2 | SEQ ID NO: 241 | SEQ ID NO: 242 |
| At.GA2 oxidase_3 | SEQ ID NO: 243 | SEQ ID NO: 244 |
| At.GA2 oxidase_4 | SEQ ID NO: 245 | SEQ ID NO: 246 |
| At.GA2 oxidase_6 | SEQ ID NO: 247 | SEQ ID NO: 248 |
| At.GA2 oxidase_7 | SEQ ID NO: 249 | SEQ ID NO: 250 |
| At.GA2 oxidase_8 | SEQ ID NO: 251 | SEQ ID NO: 252 |

A family of at least seven GA2 oxidase genes have been identified in moss (Physcomitrella patens) including Pp.GA2 oxidase_1, Pp.GA2 oxidase_2, Pp.GA2 oxidase_3, Pp.GA2 oxidase_4, Pp.GA2 oxidase_5, Pp.GA2 oxidase_6, and Pp.GA2 oxidase_7. The DNA and protein sequences by SEQ ID NO for each of these GA2 oxidase genes from moss are provided in Table 11.

TABLE 11
DNA and protein sequences for GA2 oxidase genes in moss.

| GA2 | Coding Sequence | |
|---|---|---|
| oxidase Gene | (CDS) | Protein |
| Pp.GA2 oxidase_1 | SEQ ID NO: 253 | SEQ ID NO: 254 |
| Pp.GA2 oxidase_2 | SEQ ID NO: 255 | SEQ ID NO: 256 |
| Pp.GA2 oxidase_3 | SEQ ID NO: 257 | SEQ ID NO: 258 |
| Pp.GA2 oxidase_4 | SEQ ID NO: 259 | SEQ ID NO: 260 |
| Pp.GA2 oxidase_5 | SEQ ID NO: 261 | SEQ ID NO: 262 |
| Pp.GA2 oxidase_6 | SEQ ID NO: 263 | SEQ ID NO: 264 |
| Pp.GA2 oxidase_7 | SEQ ID NO: 265 | SEQ ID NO: 266 |

A family of at least nine GA2 oxidase genes have been identified in barrel clover (Medicago truncatula) including Mt.GA2 oxidase_1, Mt.GA2 oxidase_2, Mt.GA2 oxidase_3, Mt.GA2 oxidase_4, Mt.GA2 oxidase_5, Mt.GA2 oxidase_6, Mt.GA2 oxidase_7Q Mt:GA2 oxidase_8, and Mt.GA2 oxidase_9. The DNA and protein sequences by SEQ ID NO for each of these GA2 oxidase genes from Medicago are provided in Table 12.

TABLE 12
DNA and protein sequences for GA2
oxidase genes in M. truncatula.

| GA2 | Coding Sequence | |
|---|---|---|
| oxidase Gene | (CDS) | Protein |
| Mt.GA2 oxidase_1 | SEQ ID NO: 267 | SEQ ID NO: 268 |
| Mt.GA2 oxidase_2 | SEQ ID NO: 269 | SEQ ID NO: 270 |
| Mt.GA2 oxidase_3 | SEQ ID NO: 271 | SEQ ID NO: 272 |
| Mt.GA2 oxidase_4 | SEQ ID NO: 273 | SEQ ID NO: 274 |
| Mt.GA2 oxidase_5 | SEQ ID NO: 275 | SEQ ID NO: 276 |
| Mt.GA2 oxidase_6 | SEQ ID NO: 277 | SEQ ID NO: 278 |
| Mt.GA2 oxidase_7 | SEQ ID NO: 279 | SEQ ID NO: 280 |
| Mt.GA2 oxidase_8 | SEQ ID NO: 281 | SEQ ID NO: 282 |
| Mt.GA2 oxidase_9 | SEQ ID NO: 283 | SEQ ID NO: 284 |

A family of at least four related GA2 oxidase genes have been identified in whorled honey flower (Paris polyphylla) including Ppo.GA2 oxidase_1, Ppo.GA2 oxidaseQ2, Ppo.GA2 oxidase_3, and Ppo.GA2 oxidase_4. The DNA and protein sequences by SEQ ID NO for each of these GA2 oxidase genes from honey flower are provided in Table 13.

TABLE 13
DNA and protein sequences for GA2
oxidase genes in P. polyphylla.

| GA2 | Coding Sequence | |
|---|---|---|
| oxidase Gene | (CDS) | Protein |
| Ppo.GA2 oxidase_1 | SEQ ID NO: 285 | SEQ ID NO: 286 |
| Ppo.GA2 oxidase_2 | SEQ ID NO: 287 | SEQ ID NO: 288 |
| Ppo.GA2 oxidase_3 | SEQ ID NO: 289 | SEQ ID NO: 290 |
| Ppo.GA2 oxidase_4 | SEQ ID NO: 291 | SEQ ID NO: 292 |

A family of at least eight GA2 oxidase genes have been identified in common bean (Phaseolus vulgaris) including Pv.GA2 oxidase_1, Pv.GA2 oxidase_2, Pv.GA2 oxidase_3, Pv.GA2 oxidase_4, Pv.GA2 oxidase_5, Pv.GA2 oxidase_6, Pv.GA2 oxidase_7 and Pv.GA2 oxidase_8. The DNA and protein sequences by SEQ ID NO for each of these GA2 oxidase genes from common bean are provided in Table 14.

TABLE 14
DNA and protein sequences for GA2
oxidase genes in common bean.

| GA2 | Coding Sequence | |
|---|---|---|
| oxidase Gene | (CDS) | Protein |
| Pv.GA2 oxidase_1 | SEQ ID NO: 293 | SEQ ID NO: 294 |
| Pv.GA2 oxidase_2 | SEQ ID NO: 295 | SEQ ID NO: 296 |
| Pv.GA2 oxidase_3 | SEQ ID NO: 297 | SEQ ID NO: 298 |
| Pv.GA2 oxidase_4 | SEQ ID NO: 299 | SEQ ID NO: 300 |
| Pv.GA2 oxidase_5 | SEQ ID NO: 301 | SEQ ID NO: 302 |
| Pv.GA2 oxidase_6 | SEQ ID NO: 303 | SEQ ID NO: 304 |
| Pv.GA2 oxidase_7 | SEQ ID NO: 305 | SEQ ID NO: 306 |
| Pv.GA2 oxidase_8 | SEQ ID NO: 307 | SEQ ID NO: 308 |

A family of at least seven related GA2 oxidase genes have been identified in cottonwood (Populus trichocarpa) including Pt.GA2 oxidase_1, Pt.GA2 oxidase_2, Pt.GA2 oxidase_3, Pt.GA2 oxidase_4, Pt.GA2 oxidase_5, Pt.GA2 oxidase_6, and Pt.GA2 oxidase_7. The DNA and protein sequences by SEQ ID NO for each of these GA2 oxidase genes from cottonwood are provided in Table 15.

TABLE 15
DNA and protein sequences for GA2 oxidase genes in cottonwood.

| GA2 | Coding Sequence | |
|---|---|---|
| oxidase Gene | (CDS) | Protein |
| Pt.GA2 oxidase_1 | SEQ ID NO: 309 | SEQ ID NO: 310 |
| Pt.GA2 oxidase_2 | SEQ ID NO: 311 | SEQ ID NO: 312 |
| Pt.GA2 oxidase_3 | SEQ ID NO: 313 | SEQ ID NO: 314 |
| Pt.GA2 oxidase_4 | SEQ ID NO: 315 | SEQ ID NO: 316 |
| Pt.GA2 oxidase_5 | SEQ ID NO: 317 | SEQ ID NO: 318 |
| Pt.GA2 oxidase_6 | SEQ ID NO: 319 | SEQ ID NO: 320 |
| Pt.GA2 oxidase_7 | SEQ ID NO: 321 | SEQ ID NO: 322 |

A family of at least two GA2 oxidase genes have been identified in pea (Pisum sativum) including Ps.GA2 oxidase_1, and Ps.GA2 oxidase_2. The DNA and protein sequences by SEQ ID NO for these GA2 oxidase genes from pea are provided in Table 16.

TABLE 16
DNA and protein sequences for GA2 oxidase genes in pea.

| GA2 | Coding Sequence | |
|---|---|---|
| oxidase Gene | (CDS) | Protein |
| Ps.GA2 oxidase_1 | SEQ ID NO: 323 | SEQ ID NO: 324 |
| Ps.GA2 oxidase_2 | SEQ ID NO: 325 | SEQ ID NO: 326 |

A family of at least three related GA2 oxidase genes have been identified in spinach (Spinacia oleracea) including So.GA2 oxidase_1, So.GA2 oxidase_2, and So.GA2 oxidase_3. The DNA and protein sequences by SEQ ID NO for each of these GA2 oxidase genes from spinach are provided in Table 17.

TABLE 17
DNA and protein sequences for GA2 oxidase genes in spinach.

| GA2 | Coding Sequence | |
|---|---|---|
| oxidase Gene | (CDS) | Protein |
| So.GA2 oxidase_1 | SEQ ID NO: 327 | SEQ ID NO: 328 |
| So.GA2 oxidase_2 | SEQ ID NO: 329 | SEQ ID NO: 330 |
| So.GA2 oxidase_3 | SEQ ID NO: 331 | SEQ ID NO: 332 |

According to embodiments of the present disclosure, a recombinant DNA molecule, vector or construct is provided comprising a transcribable DNA sequence encoding a GA2 oxidase mRNA and protein, wherein the GA2 oxidase protein comprises a sequence that is at least 80%, at least 85%, at least 90%, at least 95%, at least 96%, at least 97%, at least 98%, at least 99%, at least 99.5%, or 100% identical to a known GA2 oxidase protein sequence, wherein the transcribable DNA sequence is operably linked to a plant-expressible promoter, and wherein the plant is a corn plant.

According to some embodiments, a GA2 oxidase protein encoded by a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to a protein sequence from a monocot or cereal plant. According to some embodiments, a GA2 oxidase protein encoded by a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 2, 4, 6, 8, 10, 12, 14, 16, 18, 20, 22, 24 and/or 26. According to some embodiments, a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 1, 3, 5, 7, 9, 11, 13, 15, 17, 19, 21, 23 and/or 25. According to some embodiments, a GA2 oxidase protein encoded by a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 28, 30, 32, 34, 36, 38, 40, 42, 44 and/or 46. According to some embodiments, a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 27, 29, 31, 33, 35, 37, 39, 41, 43 and/or 45. According to some embodiments, a GA2 oxidase protein encoded by a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 48, 50, 52, 54, 56, 58, 60 and/or 62. According to some embodiments, a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 47, 49, 51, 53, 55, 57, 59 and/or 61. According to some embodiments, a GA2 oxidase protein encoded by a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 64, 66, 68, 70, 72, 74, 76, 78, 80, 82, 84, 86, 88, 90, 92 and/or 94. According to some embodiments, a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 63, 65, 67, 69, 71, 73, 75, 77, 79, 81, 83, 85, 87, 89, 91 and/or 93. According to some embodiments, a GA2 oxidase protein encoded by a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 96, 98, 100, 102, 104, 106, 108, 110, 112, 114, 116, 118, 120, 122 and/or 124. According to some embodiments, a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 95, 97, 99, 101, 103, 105, 107, 109, 111, 113, 115, 117, 119, 121 and/or 123. According to some embodiments, a GA2 oxidase protein encoded by a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 126, 128, 130, 132, 134, 136, 138, 140, 142, 144 and/or 146. According to some embodiments, a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 125, 127, 129, 131, 133, 135, 137, 139, 141, 143 and/or 145.

According to some embodiments, a GA2 oxidase protein encoded by a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to a protein sequence from a dicot or leguminous plant. According to some embodiments, a GA2 oxidase protein encoded by a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 148, 150, 152, 154, 156, 158, 160, 162, 164, 166, 168, 170, 172, 174, 176 and/or 178. According to some embodiments, a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 147, 149, 151, 153, 155, 157, 159, 161, 163, 165, 167, 169, 171, 173, 175 and/or 177. According to some embodiments, a GA2 oxidase protein encoded by a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 180, 182, 184, 186, 188, 190, 192, 194, 196, 198, 200, 202, 204, 206 and/or 208. According to some embodiments, a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 179, 181, 183, 185, 187, 189, 191, 193, 195, 197, 199, 201, 203, 205 and/or 207. According to some embodiments, a GA2 oxidase protein encoded by a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 210, 212, 214, 216, 218, 220, 222, 224, 226, 228, 230, 232, 234, 236 and/or 238. According to some embodiments, a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 209, 211, 213, 215, 217, 219, 221, 223, 225, 227, 229, 231, 233, 235 and/or 237. According to some embodiments, a GA2 oxidase protein encoded by a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 240, 242, 244, 246, 248, 250 and/or 252. According to some embodiments, a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 239, 241, 243, 245, 247, 249 and/or 251. According to some embodiments, a GA2 oxidase protein encoded by a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 254, 256, 258, 260, 262, 264 and/or 266. According to some embodiments, a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 253, 255, 257, 259, 261, 263 and/or 265. According to some embodiments, a GA2 oxidase protein encoded by a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 268, 270, 272, 274, 276, 278, 280, 282 and/or 284. According to some embodiments, a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 267, 269, 271, 273, 275, 277, 279, 281 and/or 283. According to some embodiments, a GA2 oxidase protein encoded by a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 286, 288, 290 and/or 292. According to some embodiments, a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 285, 287, 289 and/or 291. According to some embodiments, a GA2 oxidase protein encoded by a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 294, 296, 298, 300, 302, 304, 306 and/or 308. According to some embodiments, a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 293, 295, 297, 299, 301, 303, 305 and/or 307. According to some embodiments, a GA2 oxidase protein encoded by a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 310, 312, 314, 316, 318, 320 and/or 322. According to some embodiments, a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 309, 311, 313, 315, 317, 319 and/or 321. According to some embodiments, a GA2 oxidase protein encoded by a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 324 and/or 326. According to some embodiments, a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 323 and/or 325. According to some embodiments, a GA2 oxidase protein encoded by a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 328, 330 and/or 332. According to some embodiments, a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 327, 329 and/or 331.

According to some embodiments, a GA2 oxidase protein encoded by a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144 and/or 146. According to some embodiments, a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143 and/or 145.

According to some embodiments, a GA2 oxidase protein encoded by a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330 and/or 332. According to some embodiments, a transcribable DNA sequence of a recombinant DNA molecule, vector or construct is or comprises a sequence that is at least 80%, at least 85%, at least 90%, at least 95%, at least 96%, at least 97%, at least 98%, at least 99%, at least 99.5%, or 100% identical to one or more of SEQ ID NOs: 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329 and/or 331.

According to embodiments of the present invention, the level(s) of one or more active GAs may be reduced in the stalk or stem of a corn plant by ectopically expressing a catabolic GA2 oxidase gene to produce the short stature phenotype and resistance to lodging in transgenic plants, but without off-types in the reproductive or ear tissues of the plant.

According to embodiments of the present invention, expression of a GA2 oxidase transgene may be driven by a variety of different plant-expressible promoter types including constitutive and tissue-specific or tissue-preferred promoters, such as a vascular or leaf promoter. According to present embodiments, a recombinant DNA molecule, vector or construct for expression of a GA2 oxidase transgene in a plant is provided comprising a transcribable DNA sequence encoding a protein that is at least 80%, at least 85%, at least 90%, at least 95%, at least 96%, at least 97%, at least 98%, at least 99%, at least 99.5%, or 100% identical to a GA2 oxidase protein sequence provided herein, wherein the transcribable DNA sequence is operably linked to a plant-expressible promoter, such as a constitutive, vascular or leaf promoter. According to some embodiments, a recombinant DNA molecule, vector or construct is provided comprising a transcribable DNA sequence encoding a GA2 oxidase protein that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 2, 4, 6, 8, 10, 12, 14, 16, 18, 20, 22, 24 and/or 26, wherein the transcribable DNA sequence is operably linked to a plant-expressible promoter. According to some of these embodiments, the transcribable DNA sequence of the recombinant DNA molecule, vector or construct comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 1, 3, 5, 7, 9, 11, 13, 15, 17, 19, 21, 23 and/or 25. According to some embodiments, the plant expressible promoter is a vascular promoter. According to some embodiments, the plant expressible promoter is a vascular promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 1, 3, 5, 7, 9, 11, 13, 15, 17, 19, 21, 23 and/or 25, wherein the transcribable DNA sequence is operably linked to a plant-expressible promoter. According to some embodiments, the transcribable DNA sequence encodes a GA2 oxidase protein. According to some embodiments, the plant expressible promoter is a vascular promoter. According to some embodiments, the plant expressible promoter is a vascular promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence encoding a GA2 oxidase protein that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 28, 30, 32, 34, 36, 38, 40, 42, 44 and/or 46, wherein the transcribable DNA sequence is operably linked to a plant-expressible promoter. According to some of these embodiments, the transcribable DNA sequence of the recombinant DNA molecule, vector or construct comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 27, 29, 31, 33, 35, 37, 39, 41, 43 and/or 45. According to some embodiments, the plant expressible promoter is a vascular promoter. According to some embodiments, the plant expressible promoter is a vascular promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 27, 29, 31, 33, 35, 37, 39, 41, 43 and/or 45, wherein the transcribable DNA sequence is operably linked to a plant-expressible promoter. According to some embodiments, the transcribable DNA sequence encodes a GA2 oxidase protein. According to some embodiments, the plant expressible promoter is a vascular promoter. According to some embodiments, the plant expressible promoter is a vascular promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence encoding a GA2 oxidase protein that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 48, 50, 52, 54, 56, 58, 60 and/or 62, wherein the transcribable DNA sequence is operably linked to a plant-expressible promoter. According to some of these embodiments, the transcribable DNA sequence of the recombinant DNA molecule, vector or construct comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 47, 49, 51, 53, 55, 57, 59 and/or 61. According to some embodiments, the plant expressible promoter is a vascular promoter. According to some embodiments, the plant expressible promoter is a vascular promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 47, 49, 51, 53, 55, 57, 59 and/or 61, wherein the transcribable DNA sequence is operably linked to a plant-expressible promoter. According to some embodiments, the transcribable DNA sequence encodes a GA2 oxidase protein. According to some embodiments, the plant expressible promoter is a vascular promoter. According to some embodiments, the plant expressible promoter is a vascular promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence encoding a GA2 oxidase protein that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 64, 66, 68, 70, 72, 74, 76, 78, 80, 82, 84, 86, 88, 90, 92 and/or 94, wherein the transcribable DNA sequence is operably linked to a plant-expressible promoter. According to some of these embodiments, the transcribable DNA sequence of the recombinant DNA molecule, vector or construct comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 63, 65, 67, 69, 71, 73, 75, 77, 79, 81, 83, 85, 87, 89, 91 and/or 93. According to some embodiments, the plant expressible promoter is a vascular promoter. According to some embodiments, the plant expressible promoter is a vascular promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 63, 65, 67, 69, 71, 73, 75, 77, 79, 81, 83, 85, 87, 89, 91 and/or 93, wherein the transcribable DNA sequence is operably linked to a plant-expressible promoter. According to some embodiments, the transcribable DNA sequence encodes a GA2 oxidase protein. According to some embodiments, the plant expressible promoter is a vascular promoter. According to some embodiments, the plant expressible promoter is a vascular promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence encoding a GA2 oxidase protein that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 96, 98, 100, 102, 104, 106, 108, 110, 112, 114, 116, 118, 120, 122 and/or 124, wherein the transcribable DNA sequence is operably linked to a plant-expressible promoter. According to some of these embodiments, the transcribable DNA sequence of the recombinant DNA molecule, vector or construct comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 95, 97, 99, 101, 103, 105, 107, 109, 111, 113, 115, 117, 119, 121 and/or 123. According to some embodiments, the plant expressible promoter is a vascular promoter. According to some embodiments, the plant expressible promoter is a vascular promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 95, 97, 99, 101, 103, 105, 107, 109, 111, 113, 115, 117, 119, 121 and/or 123, wherein the transcribable DNA sequence is operably linked to a plant-expressible promoter. According to some embodiments, the transcribable DNA sequence encodes a GA2 oxidase protein. According to some embodiments, the plant expressible promoter is a vascular promoter. According to some embodiments, the plant expressible promoter is a vascular promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence encoding a GA2 oxidase protein that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 126, 128, 130, 132, 134, 136, 138, 140, 142, 144 and/or 146, wherein the transcribable DNA sequence is operably linked to a plant-expressible promoter. According to some of these embodiments, the transcribable DNA sequence of the recombinant DNA molecule, vector or construct comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 125, 127, 129, 131, 133, 135, 137, 139, 141, 143 and/or 145. According to some embodiments, the plant expressible promoter is a vascular promoter. According to some embodiments, the plant expressible promoter is a vascular promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 125, 127, 129, 131, 133, 135, 137, 139, 141, 143 and/or 145, wherein the transcribable DNA sequence is operably linked to a plant-expressible promoter. According to some embodiments, the transcribable DNA sequence encodes a GA2 oxidase protein. According to some embodiments, the plant expressible promoter is a vascular promoter. According to some embodiments, the plant expressible promoter is a vascular promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence encoding a GA2 oxidase protein that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 148, 150, 152, 154, 156, 158, 160, 162, 164, 166, 168, 170, 172, 174, 176 and/or 178, wherein the transcribable DNA sequence is operably linked to a plant-expressible promoter. According to some of these embodiments, the transcribable DNA sequence of the recombinant DNA molecule, vector or construct comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 147, 149, 151, 153, 155, 157, 159, 161, 163, 165, 167, 169, 171, 173, 175 and/or 177. According to some embodiments, the plant expressible promoter is a vascular promoter. According to some embodiments, the plant expressible promoter is a vascular promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 147, 149, 151, 153, 155, 157, 159, 161, 163, 165, 167, 169, 171, 173, 175 and/or 177, wherein the transcribable DNA sequence is operably linked to a plant-expressible promoter. According to some embodiments, the transcribable DNA sequence encodes a GA2 oxidase protein. According to some embodiments, the plant expressible promoter is a vascular promoter. According to some embodiments, the plant expressible promoter is a vascular promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence encoding a GA2 oxidase protein that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 180, 182, 184, 186, 188, 190, 192, 194, 196, 198, 200, 202, 204, 206 and/or 208, wherein the transcribable DNA sequence is operably linked to a plant-expressible promoter. According to some of these embodiments, the transcribable DNA sequence of the recombinant DNA molecule, vector or construct comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 179, 181, 183, 185, 187, 189, 191, 193, 195, 197, 199, 201, 203, 205 and/or 207. According to some embodiments, the plant expressible promoter is a vascular promoter. According to some embodiments, the plant expressible promoter is a vascular promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 179, 181, 183, 185, 187, 189, 191, 193, 195, 197, 199, 201, 203, 205 and/or 207, wherein the transcribable DNA sequence is operably linked to a plant-expressible promoter. According to some embodiments, the transcribable DNA sequence encodes a GA2 oxidase protein. According to some embodiments, the plant expressible promoter is a vascular promoter. According to some embodiments, the plant expressible promoter is a vascular promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence encoding a GA2 oxidase protein that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 210, 212, 214, 216, 218, 220, 222, 224, 226, 228, 230, 232, 234, 236 and/or 238, wherein the transcribable DNA sequence is operably linked to a plant-expressible promoter. According to some of these embodiments, the transcribable DNA sequence of the recombinant DNA molecule, vector or construct comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 209, 211, 213, 215, 217, 219, 221, 223, 225, 227, 229, 231, 233, 235 and/or 237. According to some embodiments, the plant expressible promoter is a vascular promoter. According to some embodiments, the plant expressible promoter is a vascular promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 209, 211, 213, 215, 217, 219, 221, 223, 225, 227, 229, 231, 233, 235 and/or 237, wherein the transcribable DNA sequence is operably linked to a plant-expressible promoter. According to some embodiments, the transcribable DNA sequence encodes a GA2 oxidase protein. According to some embodiments, the plant expressible promoter is a vascular promoter. According to some embodiments, the plant expressible promoter is a vascular promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence encoding a GA2 oxidase protein that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 240, 242, 244, 246, 248, 250 and/or 252, wherein the transcribable DNA sequence is operably linked to a plant-expressible promoter. According to some of these embodiments, the transcribable DNA sequence of the recombinant DNA molecule, vector or construct comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 239, 241, 243, 245, 247, 249 and/or 251. According to some embodiments, the plant expressible promoter is a vascular promoter. According to some embodiments, the plant expressible promoter is a vascular promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 239, 241, 243, 245, 247, 249 and/or 251, wherein the transcribable DNA sequence is operably linked to a plant-expressible promoter. According to some embodiments, the transcribable DNA sequence encodes a GA2 oxidase protein. According to some embodiments, the plant expressible promoter is a vascular promoter. According to some embodiments, the plant expressible promoter is a vascular promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence encoding a GA2 oxidase protein that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 254, 256, 258, 260, 262, 264 and/or 266, wherein the transcribable DNA sequence is operably linked to a plant-expressible promoter. According to some of these embodiments, the transcribable DNA sequence of the recombinant DNA molecule, vector or construct comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 253, 255, 257, 259, 261, 263 and/or 265. According to some embodiments, the plant expressible promoter is a vascular promoter. According to some embodiments, the plant expressible promoter is a vascular promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 253, 255, 257, 259, 261, 263 and/or 265, wherein the transcribable DNA sequence is operably linked to a plant-expressible promoter. According to some embodiments, the transcribable DNA sequence encodes a GA2 oxidase protein. According to some embodiments, the plant expressible promoter is a vascular promoter. According to some embodiments, the plant expressible promoter is a vascular promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence encoding a GA2 oxidase protein that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 268, 270, 272, 274, 276, 278, 280, 282 and/or 284, wherein the transcribable DNA sequence is operably linked to a plant-expressible promoter. According to some of these embodiments, the transcribable DNA sequence of the recombinant DNA molecule, vector or construct comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 267, 269, 271, 273, 275, 277, 279, 281 and/or 283. According to some embodiments, the plant expressible promoter is a vascular promoter. According to some embodiments, the plant expressible promoter is a vascular promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 267, 269, 271, 273, 275, 277, 279, 281 and/or 283, wherein the transcribable DNA sequence is operably linked to a plant-expressible promoter. According to some embodiments, the transcribable DNA sequence encodes a GA2 oxidase protein. According to some embodiments, the plant expressible promoter is a vascular promoter. According to some embodiments, the plant expressible promoter is a vascular promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence encoding a GA2 oxidase protein that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 286, 288, 290 and/or 292, wherein the transcribable DNA sequence is operably linked to a plant-expressible promoter. According to some of these embodiments, the transcribable DNA sequence of the recombinant DNA molecule, vector or construct comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 285, 287, 289 and/or 291. According to some embodiments, the plant expressible promoter is a vascular promoter. According to some embodiments, the plant expressible promoter is a vascular promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 285, 287, 289 and/or 291, wherein the transcribable DNA sequence is operably linked to a plant-expressible promoter. According to some embodiments, the transcribable DNA sequence encodes a GA2 oxidase protein. According to some embodiments, the plant expressible promoter is a vascular promoter. According to some embodiments, the plant expressible promoter is a vascular promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence encoding a GA2 oxidase protein that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 294, 296, 298, 300, 302, 304, 306 and/or 308, wherein the transcribable DNA sequence is operably linked to a plant-expressible promoter. According to some of these embodiments, the transcribable DNA sequence of the recombinant DNA molecule, vector or construct comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 293, 295, 297, 299, 301, 303, 305 and/or 307. According to some embodiments, the plant expressible promoter is a vascular promoter. According to some embodiments, the plant expressible promoter is a vascular promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 293, 295, 297, 299, 301, 303, 305 and/or 307, wherein the transcribable DNA sequence is operably linked to a plant-expressible promoter. According to some embodiments, the transcribable DNA sequence encodes a GA2 oxidase protein. According to some embodiments, the plant expressible promoter is a vascular promoter. According to some embodiments, the plant expressible promoter is a vascular promoter comprising a sequence that is at least 70%, at least \75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence encoding a GA2 oxidase protein that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 310, 312, 314, 316, 318, 320 and/or 322, wherein the transcribable DNA sequence is operably linked to a plant-expressible promoter. According to some of these embodiments, the transcribable DNA sequence of the recombinant DNA molecule, vector or construct comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 309, 311, 313, 315, 317, 319 and/or 321. According to some embodiments, the plant expressible promoter is a vascular promoter. According to some embodiments, the plant expressible promoter is a vascular promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 309, 311, 313, 315, 317, 319 and/or 321, wherein the transcribable DNA sequence is operably linked to a plant-expressible promoter. According to some embodiments, the transcribable DNA sequence encodes a GA2 oxidase protein. According to some embodiments, the plant expressible promoter is a vascular promoter. According to some embodiments, the plant expressible promoter is a vascular promoter comprising a sequence that is at least 70%, at least \75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence encoding a GA2 oxidase protein that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 324 and/or 326, wherein the transcribable DNA sequence is operably linked to a plant-expressible promoter. According to some of these embodiments, the transcribable DNA sequence of the recombinant DNA molecule, vector or construct comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 323 and/or 325. According to some embodiments, the plant expressible promoter is a vascular promoter. According to some embodiments, the plant expressible promoter is a vascular promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 323 and/or 325, wherein the transcribable DNA sequence is operably linked to a plant-expressible promoter. According to some embodiments, the transcribable DNA sequence encodes a GA2 oxidase protein. According to some embodiments, the plant expressible promoter is a vascular promoter. According to some embodiments, the plant expressible promoter is a vascular promoter comprising a sequence that is at least 70%, at least \75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence encoding a GA2 oxidase protein that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 328, 330 and/or 332, wherein the transcribable DNA sequence is operably linked to a plant-expressible promoter. According to some of these embodiments, the transcribable DNA sequence of the recombinant DNA molecule, vector or construct comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 327, 329 and/or 331. According to some embodiments, the plant expressible promoter is a vascular promoter. According to some embodiments, the plant expressible promoter is a vascular promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to some embodiments, a recombinant DNA molecule, vector or construct is provided comprising a transcribable DNA sequence that comprises or consists of a sequence that is at least 80%, at least 85%, at least 90%, at least 95%, at least 96%, at least 97%, at least 98%, at least 99%, at least 99.5%, or 100% identical to one or more of SEQ ID NOs: 327, 329 and/or 331, wherein the transcribable DNA sequence is operably linked to a plant-expressible promoter. According to some embodiments, the transcribable DNA sequence encodes a GA2 oxidase protein. According to some embodiments, the plant expressible promoter is a vascular promoter. According to some embodiments, the plant expressible promoter is a vascular promoter comprising a sequence that is at least 70%, at least \75%, at least 80%, at least 85%, at least 90%, at least 95%, at least 96%, at least 97%, at least 98%, at least 99%, at least 99.5%, or 100% identical to one or more of SEQ ID NOs: 333, 334, 335, 336, 337, 338 and/or 339, or a functional portion of any of the foregoing. According to some embodiments, the plant expressible promoter is a RTBV promoter (e.g., a promoter comprising the RTBV (SEQ ID NO: 333) or truncated RTBV (SEQ ID NO: 334) sequence) or a promoter comprising a sequence that is at least 70%, at least 75%, at least 80%, at least 85%, at least 90%, at least 95%, at least 96%, at least 97%, at least 98%, at least 99%, at least 99.5%, or 100% identical to SEQ ID NO: 333 and/or 334. According to some embodiments, the plant expressible promoter is a leaf promoter. According to some embodiments, the plant expressible promoter is a leaf promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 340, 341 and/or 342, or a functional portion of any of the foregoing. According to some embodiments, the plant expressible promoter is a constitutive promoter. According to some embodiments, the plant expressible promoter is a constitutive promoter comprising a sequence that is at least 70%, at least 75%, at least 80%, at least 85%, at least 90%, at least 95%, at least 96%, at least 97%, at least 98%, at least 99%, at least 99.5%, or 100% identical to one or more of SEQ ID NOs: 343, 344, 345, 346, 347, 348, 349, 350 and/or 351, or a functional portion of any of the foregoing.

According to many embodiments, a modified or transgenic corn plant is provided comprising and/or transformed with a recombinant DNA construct comprising a transcribable DNA sequence encoding a GA2 oxidase protein as provided herein. According to some embodiments, a modified or transgenic corn plant is provided that is transformed with a recombinant DNA construct comprising a transcribable DNA sequence encoding a GA2 oxidase mRNA and protein, wherein the transcribable DNA sequence is operably linked to a plant-expressible promoter, wherein the GA2 oxidase mRNA and/or protein is identical to an endogenous GA2 oxidase protein, and wherein the expression level of the GA2 oxidase mRNA and/or protein is increased in one or more plant tissue(s) of the modified or transgenic plant as compared to a wild type or control plant, such as increased in one or more vascular and/or leaf tissue(s) of the modified or transgenic plant, such as by at least 5%, at least 10%, at least 20%, at least 25%, at least 30%, at least 40%, at least 50%, at least 60%, at least 70%, at least 75%, at least 80%, at least 90%, or 100%, as compared to a wild type or control plant.

According to present embodiments, a modified or transgenic corn plant is provided comprising a recombinant DNA construct comprising a transcribable DNA sequence encoding a GA2 oxidase protein as provided herein, wherein the level of one or more active GAs, such as GAL, GA3, GA4, and/or GA7, is reduced or lowered in one or more plant tissue(s), such as one or more stem, internode, vascular and/or leaf tissue(s) or one or more stem and/or internode tissue(s), of the modified or transgenic plant, such as by at least 5%, at least 10%, at least 20%, at least 25%, at least 30%, at least 40%, at least 50%, at least 60%, at least 70%, at least 75%, at least 80%, at least 90%, or 100%, as compared to a wild type or control plant.

According to many embodiments, a modified or transgenic plant is provided that is transformed with a recombinant DNA construct comprising a transcribable DNA sequence encoding a GA2 oxidase protein as provided herein, wherein the transcribable DNA sequence is operably linked to a constitutive promoter or a tissue-specific or tissue-preferred promoter, such as a vascular promoter or a leaf promoter, and wherein the modified or transgenic plant has one or more of the following traits: a semi-dwarf or reduced plant height or stature, decreased stem internode length, increased lodging resistance, and/or increased stem or stalk diameter. Such a modified or transgenic plant may not have any significant reproductive off-types. A modified or transgenic plant may have one or more of the following additional traits: reduced green snap, deeper roots, increased leaf area, earlier canopy closure, higher stomatal conductance, lower ear height, increased foliar water content, improved drought tolerance, increased nitrogen use efficiency, increased water use efficiency, reduced anthocyanin content and anthocyanin area in leaves under normal and/or nitrogen or water limiting stress conditions, increased ear weight, increased kernel number, increased kernel weight, increased yield, and/or increased harvest index. According to many embodiments, the level of one or more active GAs, such as GA1, GA3, GA4, and/or GA7, is/are reduced or lowered in one or more plant tissue(s), such as one or more stem, internode, vascular and/or leaf tissue(s), or one or more stem and/or internode tissue(s), of the modified or transgenic plant, such as by at least 5%, at least 10%, at least 20%, at least 25%, at least 30%, at least 40%, at least 50%, at least 60%, at least 70%, at least 75%, at least 80%, at least 90%, or 100%, as compared to a wild type or control plant.

A recombinant DNA molecule, construct or vector of the present disclosure may comprise a transcribable DNA sequence encoding a GA2 oxidase as provided herein, wherein the transcribable DNA sequence is operatively linked to a plant-expressible promoter, such as a constitutive or vascular and/or leaf promoter. In addition to its associated promoter, a transcribable DNA sequence encoding a GA2 oxidase may also be operatively linked to one or more additional regulatory element(s), such as an enhancer(s), leader, transcription start site (TSS), linker, 5′ and 3′ untranslated region(s) (UTRs), intron(s), polyadenylation signal, termination region or sequence, etc., that are suitable, necessary or preferred for strengthening, regulating or allowing expression of the transcribable DNA sequence in a corn plant cell. Such additional regulatory element(s) may be optional and/or used to enhance or optimize expression of the transgene or transcribable DNA sequence. As provided herein, an “enhancer” may be distinguished from a “promoter” in that an enhancer typically lacks a transcription start site, TATA box, or equivalent sequence and is thus insufficient alone to drive transcription. As used herein, a “leader” may be defined generally as the DNA sequence of the 5′-UTR of a gene (or transgene) between the transcription start site (TSS) and 5′ end of the transcribable DNA sequence or protein coding sequence start site of the transgene.

According to further embodiments, methods are provided for transforming a plant cell, tissue or explant with a recombinant DNA molecule, vector or construct comprising a transcribable DNA sequence or transgene operably linked to a plant-expressible promoter to produce a transgenic plant. The transcribable DNA sequence may encode a GA2 oxidase as provided herein. Numerous methods for transforming chromosomes or plastids in a plant cell with a recombinant DNA molecule, vector or construct are known in the art, which may be used according to method embodiments of the present invention to produce a transgenic plant cell and plant. Any suitable method or technique for transformation of a plant cell known in the art may be used according to present methods. Effective methods for transformation of plants include bacterially mediated transformation, such as Agrobacterium-mediated or Rhizobium-mediated transformation, and microprojectile or particle bombardment-mediated transformation. A variety of methods are known in the art for transforming explants with a transformation vector via bacterially mediated transformation or microprojectile or particle bombardment and then subsequently culturing, etc., those explants to regenerate or develop transgenic plants. Other methods for plant transformation, such as microinjection, electroporation, vacuum infiltration, pressure, sonication, silicon carbide fiber agitation, PEG-mediated transformation, etc., are also known in the art.

Methods of transforming plant cells and explants are well known by persons of ordinary skill in the art. Methods for transforming plant cells by microprojectile bombardment with particles coated with recombinant DNA are provided, for example, in U.S. Pat. Nos. 5,550,318; 5,538,880 6,160,208; 6,399,861; and 6,153,812, and Agrobacterium-mediated transformation is described, for example, in U.S. Pat. Nos. 5,159,135; 5,824,877; 5,591,616; 6,384,301; 5,750,871; 5,463,174; and 5,188,958, all of which are incorporated herein by reference. Additional methods for transforming plants can be found in, for example, Compendium of Transgenic Crop Plants (2009) Blackwell Publishing. Any suitable method of plant transformation known or later developed in the art can be used to transform a plant cell or explant with any of the nucleic acid molecules, constructs or vectors provided herein.

Transgenic plants produced by transformation methods may be chimeric or non-chimeric for the transformation event depending on the methods and explants used. Methods are further provided for expressing a GA2 oxidase transgene in one or more plant cells or tissues under the control of a plant-expressible promoter, such as a constitutive, tissue-specific, tissue-preferred, vascular and/or leaf promoter as provided herein. Such methods may be used to create transgenic corn plants having a shorter, semi-dwarf stature, reduced internode length, increased stalk/stem diameter, and/or improved lodging resistance. Such transgenic corn plants may further have other traits that may be beneficial for yield, such as reduced green snap, deeper roots, increased leaf area, earlier canopy closure, improved drought tolerance, increased nitrogen use efficiency, increased water use efficiency, higher stomatal conductance, lower ear height, increased foliar water content, reduced anthocyanin content and/or area in leaves under normal or nitrogen or water limiting stress conditions, increased ear weight, increased seed or kernel number, increased seed or kernel weight, increased yield, and/or increased harvest index, relative to a wild type or control plant. As used herein, “harvest index” refers to the mass of the harvested grain divided by the total mass of the above-ground biomass of the plant over a harvested area.

Transgenic plants expressing a GA2 oxidase transgene may have an earlier canopy closure (e.g., approximately one day earlier, or 12-48 hours, 12-36 hours, 18-36 hours, or about 24 hours earlier canopy closure) than a wild type or control plant. Although transgenic plants expressing a GA2 oxidase transgene may have a lower ear height than a wild type or control plant, the height of the ear may generally be at least 18 inches above the ground. Transgenic plants expressing a GA2 oxidase may have greater biomass and/or leaf area during one or more late vegetative stages (e.g., V8-V12) than a wild type or control plant. Transgenic plants expressing a GA2 oxidase may have deeper roots during later vegetative stages when grown in the field, than a wild type or control plant, which may be due to an increased root front velocity. These transgenic plants may reach a depth 90 cm below ground sooner (e.g., 5-25 days sooner, 5-20 days sooner, 5-15 days sooner, 10-25 days sooner, or 15-25 days sooner, or about 5, 10, 15, 20 Or 25 days sooner) than a wild type or control plant, which may occur by or prior to the vegetative to reproductive transition of the plant (e.g., by V16/R1 at about 50 days after planting as opposed to about 70 days after planting for control plants).

Recipient cell(s) or explant or cellular targets for transformation include, but are not limited to, a seed cell, a fruit cell, a leaf cell, a cotyledon cell, a hypocotyl cell, a meristem cell, an embryo cell, an endosperm cell, a root cell, a shoot cell, a stem cell, a pod cell, a flower cell, an inflorescence cell, a stalk cell, a pedicel cell, a style cell, a stigma cell, a receptacle cell, a petal cell, a sepal cell, a pollen cell, an anther cell, a filament cell, an ovary cell, an ovule cell, a pericarp cell, a phloem cell, a bud cell, a callus cell, a chloroplast, a stomatal cell, a trichome cell, a root hair cell, a storage root cell, or a vascular tissue cell, a seed, embryo, meristem, cotyledon, hypocotyl, endosperm, root, shoot, stem, node, callus, cell suspension, protoplast, flower, leaf, pollen, anther, ovary, ovule, pericarp, bud, and/or vascular tissue, or any transformable portion of any of the foregoing. For plant transformation, any target cell(s), tissue(s), explant(s), etc., that may be used to receive a recombinant DNA transformation vector or molecule of the present disclosure may be collectively be referred to as an “explant” for transformation. Preferably, a transformable or transformed explant cell or tissue may be further developed or regenerated into a plant. Any cell or explant from which a fertile plant can be grown or regenerated is contemplated as a useful recipient cell or explant for practice of this disclosure (i.e., as a target explant for transformation). Callus can be initiated or created from various tissue sources, including, but not limited to, embryos or parts of embryos, non-embryonic seed tissues, seedling apical meristems, microspores, and the like. Any cells that are capable of proliferating as callus may serve as recipient cells for transformation. Transformation methods and materials for making transgenic plants (e.g., various media and recipient target cells or explants and methods of transformation and subsequent regeneration of into transgenic plants) are known in the art.

Transformation of a target plant material or explant may be practiced in tissue culture on nutrient media, for example a mixture of nutrients that allow cells to grow in vitro or cell culture. Transformed explants, cells or tissues may be subjected to additional culturing steps, such as callus induction, selection, regeneration, etc., as known in the art. Transformation may also be carried out without creation or use of a callus tissue. Transformed cells, tissues or explants containing a recombinant DNA sequence insertion or event may be grown, developed or regenerated into transgenic plants in culture, plugs, or soil according to methods known in the art. Transgenic plants may be further crossed to themselves or other plants to produce transgenic seeds and progeny. A transgenic plant may also be prepared by crossing a first plant comprising the recombinant DNA sequence or transformation event with a second plant lacking the insertion. For example, a recombinant DNA construct or sequence may be introduced into a first plant line that is amenable to transformation, which may then be crossed with a second plant line to introgress the recombinant DNA construct or sequence into the second plant line. Progeny of these crosses can be further back crossed into the more desirable line multiple times, such as through 6 to 8 generations or back crosses, to produce a progeny plant with substantially the same genotype as the original parental line, but for the introduction of the recombinant DNA construct or sequence.

A transgenic plant, plant part, cell, or explant provided herein may be of an elite variety or an elite line. An elite variety or an elite line refers to a variety that has resulted from breeding and selection for superior agronomic performance. A transgenic plant, cell, or explant provided herein may be a hybrid plant, cell, or explant. As used herein, a “hybrid” is created by crossing two plants from different varieties, lines, inbreds, or species, such that the progeny comprises genetic material from each parent. Skilled artisans recognize that higher order hybrids can be generated as well. For example, a first hybrid can be made by crossing Variety A with Variety B to create a A×B hybrid, and a second hybrid can be made by crossing Variety C with Variety D to create an C×D hybrid. The first and second hybrids can be further crossed to create the higher order hybrid (A×B)×(C×D) comprising genetic information from all four parent varieties.

According to some embodiments, a recombinant DNA construct or vector may comprise two or more expression elements or cassettes that may be stacked together in a construct or vector either in tandem in a single expression cassette or separately in two or more expression cassettes. A recombinant DNA construct or vector may comprise either a single expression cassette comprising a transcribable DNA sequence that encodes a GA2 oxidase mRNA and protein or two or more expression cassettes comprising two or more transcribable DNA sequences that encode two or more GA2 oxidase mRNAs and proteins, including at least a first GA2 oxidase mRNA and protein and a second GA2 oxidase mRNA and protein, wherein the two or more transcribable DNA sequences, GA2 oxidase mRNAs and/or GA2 oxidase proteins are the same or different, and wherein each transcribable DNA sequence is operably linked to a plant-expressible promoter. The plant-expressible promoter may be a constitutive promoter, or a tissue-specific or tissue-preferred promoter, as provided herein. If two or more transcribable DNA sequences are present in a recombinant DNA construct or vector or a modified or transgenic plant, plant part, cell, or explant, each transcribable DNA sequence may be operably linked to the same or different plant-expressible promoters.

According to other embodiments, a recombinant DNA construct or vector may comprise two or more expression cassettes including a first expression cassette and a second expression cassette, wherein the first expression cassette comprises a first transcribable DNA sequence operably linked to a first plant-expressible promoter, and the second expression cassette comprises a second transcribable DNA sequence operably linked to a second plant-expressible promoter, wherein the first transcribable DNA sequence encodes a first GA2 oxidase and the second transcribable DNA sequence encodes a second GA2 oxidase. The first and second plant-expressible promoters may each be a constitutive promoter, or a tissue-specific or tissue-preferred promoter, as provided herein, and the first and second plant-expressible promoters may be the same or different promoters.

According to other embodiments, two or more constructs, expression cassettes or transgenes encoding one or more GA2 oxidase proteins may be combined in a modified plant by crossing two or more plants together in one or more generations to produce a modified plant having a desired combination of the constructs, expression cassettes or transgenes. According to these embodiments, a first modified plant comprising a first construct, expression cassette or transgene encoding a first GA2 oxidase protein may be crossed to a second modified plant comprising a second construct, expression cassette or transgene encoding a second GA2 oxidase protein, such that a modified progeny plant may be made comprising the first construct, expression cassette or transgene and the second construct, expression cassette or transgene. Alternatively, a modified plant comprising two or more constructs, expression cassettes or transgenes encoding two or more GA2 oxidase proteins may be made by (i) co-transforming a first construct, expression cassette or transgene and a second construct, expression cassette or transgene (each encoding a GA2 oxidase protein) in the same or different transformation molecules or vectors, (ii) transforming a modified plant with a second construct, expression cassette or transgene in a transformation molecule or vector, wherein the modified plant already comprises a first construct, expression cassette or transgene, or (iii) transforming a plant with a first construct, expression cassette or transgene in a first transformation molecule or vector, and then transforming the plant with a second construct, expression cassette or transgene in a second transformation molecule or vector.

According to embodiments of the present disclosure, modified plants are provided comprising two or more constructs comprising GA2 oxidase transgene(s) including a first recombinant DNA construct and a second recombinant DNA construct, wherein the first recombinant DNA construct comprises a first transcribable DNA sequence encoding a first GA2 oxidase mRNA and protein, and the second recombinant DNA construct comprises a second transcribable DNA sequence encoding a second GA2 oxidase mRNA and protein. The first and second recombinant DNA constructs may be stacked in a single vector and transformed into a plant as a single event, or present in separate vectors or constructs that may be transformed as separate events. According to some embodiments, the first and second GA2 oxidase transgenes may be the same or different GA oxidase gene(s).

A recombinant DNA molecule, construct or expression cassette of the present disclosure may comprise or be included within a DNA transformation vector or molecule for use in transformation of a target plant cell, tissue or explant. Such a transformation vector may generally comprise sequences or elements necessary or beneficial for effective transformation in addition to at least one transgene, expression cassette and/or transcribable DNA sequence encoding a GA2 oxidase. For Agrobacterium-mediated, Rhizobia-mediated or other bacteria-mediated transformation, the transformation vector may comprise an engineered transfer DNA (or T-DNA) segment or region having two border sequences, a left border (LB) and a right border (RB), flanking at least a transcribable DNA sequence or transgene, such that insertion of the T-DNA into the plant genome will create a transformation event for the transcribable DNA sequence, transgene or expression cassette. Thus, a transcribable DNA sequence, transgene or expression cassette encoding a GA2 oxidase may be located between the left and right borders of the T-DNA, perhaps along with an additional transgene(s) or expression cassette(s), such as a plant selectable marker transgene and/or other gene(s) of agronomic interest that may confer a trait or phenotype of agronomic interest to a plant. According to alternative embodiments, the transcribable DNA sequence, transgene or expression cassette encoding a GA2 oxidase and the plant selectable marker transgene (or other gene of agronomic interest) may be present in separate T-DNA segments on the same or different recombinant DNA molecule(s), such as for co-transformation. A transformation vector or construct may further comprise prokaryotic maintenance elements, which may be located in the vector outside of the T-DNA region(s).

A plant selectable marker transgene in a transformation vector or construct of the present disclosure may be used to assist in the selection of transformed cells or tissue due to the presence of a selection agent, such as an antibiotic or herbicide, wherein the plant selectable marker transgene provides tolerance or resistance to the selection agent. Thus, the selection agent may bias or favor the survival, development, growth, proliferation, etc., of transformed cells expressing the plant selectable marker gene, such as to increase the proportion of transformed cells or tissues in the R₀ plant. Commonly used plant selectable marker genes include, for example, those conferring tolerance or resistance to antibiotics, such as kanamycin and paromomycin (nptII), hygromycin B (aph IV), streptomycin or spectinomycin (aadA) and gentamycin (aac3 and aacC4), or those conferring tolerance or resistance to herbicides such as glufosinate (bar or pat), dicamba (DMO) and glyphosate (aroA or EPSPS). Plant screenable marker genes may also be used, which provide an ability to visually screen for transformants, such as luciferase or green fluorescent protein (GFP), or a gene expressing a beta glucuronidase or uidA gene (GUS) for which various chromogenic substrates are known. In some embodiments, a vector or polynucleotide provided herein comprises at least one selectable marker gene selected from the group consisting of nptII, aph IV, aadA, aac3, aacC4, bar, pat, DMO, EPSPS, aroA, GFP, and GUS. Plant transformation may also be carried out in the absence of selection during one or more steps or stages of culturing, developing or regenerating transformed explants, tissues, plants and/or plant parts.

According to present embodiments, methods for transforming a plant cell, tissue or explant with a recombinant DNA molecule or construct may further include site-directed or targeted integration. According to these methods, a portion of a recombinant DNA donor template molecule (i.e., an insertion sequence) may be inserted or integrated at a desired target site or locus within the plant genome. The insertion sequence of the donor template may comprise a transgene or construct, such as a transgene or transcribable DNA sequence encoding a GA2 oxidase mRNA and protein. The donor template may also have one or two homology arms flanking the insertion sequence to promote the targeted insertion event through homologous recombination and/or homology-directed repair. Each homology arm may be at least 70%, at least 75%, at least 80%, at least 85%, at least 90%, at least 95%, at least 96%, at least 97%, at least 99% or 100% identical or complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a target DNA sequence within the genome of a corn plant. Thus, a recombinant DNA molecule of the present disclosure may comprise a donor template for site-directed or targeted integration of a transgene or construct, such as a transgene or transcribable DNA sequence encoding a GA2 oxidase, into the genome of a plant.

As used herein, a “targeted genome editing technique” for site-directed integration refers to any method, protocol, or technique that allows the precise and/or targeted insertion of a recombinant DNA construct, transgene and/or expression cassette comprising a transcribable DNA sequence encoding a GA2 oxidase mRNA and protein into a target site in a genome of a plant using a site-specific nuclease, such as a meganuclease, a zinc-finger nuclease (ZFN), an RNA-guided endonuclease (e.g., the CRISPR/Cas9 system), a TALE-endonuclease (TALEN), a recombinase, or a transposase, wherein the recombinant DNA construct, transgene and/or expression cassette is provided by an insertion sequence of a DNA donor template or molecule.

Any site or locus within the genome of a plant may potentially be chosen for site-directed integration of a transgene, construct or transcribable DNA sequence provided herein. For site-directed integration, a double-strand break (DSB) or nick may first be made at a selected genomic locus with a site-specific nuclease, such as, for example, a zinc-finger nuclease, an engineered or native meganuclease, a TALE-endonuclease, or an RNA-guided endonuclease (e.g., Cas9 or Cpf1). Any method known in the art for site-directed integration may be used. In the presence of a donor template molecule with an insertion sequence, the DSB or nick may then be repaired by homologous recombination between homology arm(s) of the donor template and the plant genome, or by non-homologous end joining (NHEJ), resulting in site-directed integration of the insertion sequence into the plant genome to create the targeted insertion event at the site of the DSB or nick. Thus, site-specific insertion or integration of a transgene, construct or sequence may be achieved.

A site-specific nuclease provided herein may be selected from the group consisting of a zinc-finger nuclease (ZFN), a meganuclease, an RNA-guided endonuclease, a TALE-endonuclease (TALEN), a recombinase, a transposase, or any combination thereof. See, e.g., Khandagale, K. et al., “Genome editing for targeted improvement in plants,” Plant Biotechnol Rep 10: 327-343 (2016); and Gaj, T. et al., “ZFN, TALEN and CRISPR/Cas-based methods for genome engineering,” Trends Biotechnol. 31(7): 397-405 (2013), the contents and disclosures of which are incorporated herein by reference. A recombinase may be a serine recombinase attached to a DNA recognition motif, a tyrosine recombinase attached to a DNA recognition motif or other recombinase enzyme known in the art. A recombinase or transposase may be a DNA transposase or recombinase attached to a DNA binding domain. A tyrosine recombinase attached to a DNA recognition motif may be selected from the group consisting of a Cre recombinase, a Flp recombinase, and a Tnp1 recombinase. According to some embodiments, a Cre recombinase or a Gin recombinase provided herein is tethered to a zinc-finger DNA binding domain. In another embodiment, a serine recombinase attached to a DNA recognition motif provided herein is selected from the group consisting of a PhiC31 integrase, an R4 integrase, and a TP-901 integrase. In another embodiment, a DNA transposase attached to a DNA binding domain provided herein is selected from the group consisting of a TALE-piggyBac and TALE-Mutator.

According to embodiments of the present disclosure, an RNA-guided endonuclease may be selected from the group consisting of Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, CasX, CasY, and homologs or modified versions thereof, Argonaute (non-limiting examples of Argonaute proteins include Thermus thermophilus Argonaute (TtAgo), Pyrococcus furiosus Argonaute (PfAgo), Natronobacterium gregoryi Argonaute (NgAgo) and homologs or modified versions thereof. According to some embodiments, an RNA-guided endonuclease may be a Cas9 or Cpf1 enzyme.

In an aspect, a site-specific nuclease provided herein is selected from the group consisting of a zinc-finger nuclease, a meganuclease, an RNA-guided nuclease, a TALE-nuclease, a recombinase, a transposase, or any combination thereof. In another aspect, a site-specific nuclease provided herein is selected from the group consisting of a Cas9 or a Cpf1. In another aspect, a site-specific nuclease provided herein is selected from the group consisting of a Cas1, a CasIB, a Cas2, a Cas3, a Cas4, a Cas5, a Cas6, a Cas7, a Cas8, a Cas9, a Cas10, a Csy1, a Csy2, a Csy3, a Cse1, a Cse2, a Csc1, a Csc2, a Csa5, a Csn2, a Csm2, a Csm3, a Csm4, a Csm5, a Csm6, a Cmr1, a Cmr3, a Cmr4, a Cmr5, a Cmr6, a Csb1, a Csb2, a Csb3, a Csx17, a Csx14, a Csx10, a Csx16, a CsaX, a Csx3, a Csx1, a Csx15, a Csf1, a Csf2, a Csf3, a Csf4, a Cpf1, CasX, CasY, a homolog thereof, or a modified version thereof. In another aspect, an RNA-guided nuclease provided herein is selected from the group consisting of a Cas9 or a Cpf1. In another aspect, an RNA guided nuclease provided herein is selected from the group consisting of a Cas1, a Cas1B, a Cas2, a Cas3, a Cas4, a Cas5, a Cas6, a Cas7, a Cas8, a Cas9, a Cas10, a Csy1, a Csy2, a Csy3, a Cse1, a Cse2, a Csc1, a Csc2, a Csa5, a Csn2, a Csm2, a Csm3, a Csm4, a Csm5, a Csm6, a Cmr1, a Cmr3, a Cmr4, a Cmr5, a Cmr6, a Csb1, a Csb2, a Csb3, a Csx17, a Csx14, a Csx10, a Csx16, a CsaX, a Csx3, a Csx1, a Csx15, a Csf1, a Csf2, a Csf3, a Csf4, a Cpf1, CasX, CasY, a homolog thereof, or a modified version thereof. In another aspect, a method and/or a composition provided herein comprises at least one, at least two, at least three, at least four, at least five, at least six, at least seven, at least eight, at least nine, or at least ten site-specific nucleases. In yet another aspect, a method and/or a composition provided herein comprises at least one, at least two, at least three, at least four, at least five, at least six, at least seven, at least eight, at least nine, or at least ten polynucleotides encoding at least one, at least two, at least three, at least four, at least five, at least six, at least seven, at least eight, at least nine, or at least ten site-specific nucleases.

For RNA-guided endonucleases, a guide RNA (gRNA) molecule is further provided to direct the endonuclease to a target site in the genome of the plant via base-pairing or hybridization to cause a DSB or nick at or near the target site. The gRNA may be transformed or introduced into a plant cell or tissue (perhaps along with a nuclease, or nuclease-encoding DNA molecule, construct or vector) as a gRNA molecule, or as a recombinant DNA molecule, construct or vector comprising a polynucleotide or transcribable DNA sequence encoding the guide RNA operably linked to a plant-expressible promoter. As understood in the art, a “guide RNA” may comprise, for example, a CRISPR RNA (crRNA), a single-chain guide RNA (sgRNA), or any other RNA molecule that may guide or direct an endonuclease to a specific target site in the genome. A “single-chain guide RNA” (or “sgRNA”) is a RNA molecule comprising a crRNA covalently linked a tracrRNA by a linker sequence, which may be expressed as a single RNA transcript or molecule. The guide RNA comprises a guide or targeting sequence that is identical or complementary to a target site within the plant genome, such as at or near a GA oxidase gene. A protospacer-adjacent motif (PAM) may be present in the genome immediately adjacent and upstream to the 5′ end of the genomic target site sequence complementary to the targeting sequence of the guide RNA—i.e., immediately downstream (3′) to the sense (+) strand of the genomic target site (relative to the targeting sequence of the guide RNA) as known in the art. See, e.g., Wu, X. et al., “Target specificity of the CRISPR-Cas9 system,” Quant Biol. 2(2): 59-70 (2014), the content and disclosure of which is incorporated herein by reference. The genomic PAM sequence on the sense (+) strand adjacent to the target site (relative to the targeting sequence of the guide RNA) may comprise 5′-NGG-3′. However, the corresponding sequence of the guide RNA (i.e., immediately downstream (3′) to the targeting sequence of the guide RNA) may generally not be complementary to the genomic PAM sequence. The guide RNA may typically be a non-coding RNA molecule that does not encode a protein. The guide sequence of the guide RNA may be at least 10 nucleotides in length, such as 12-40 nucleotides, 12-30 nucleotides, 12-20 nucleotides, 12-35 nucleotides, 12-30 nucleotides, 15-30 nucleotides, 17-30 nucleotides, or 17-25 nucleotides in length, or about 12, 13, 14, 15, 16, 17, 18, 19, 20, 21, 22, 23, 24, 25 or more nucleotides in length. The guide sequence may be at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of a DNA sequence at the genomic target site. As used herein, the term “consecutive” in reference to a polynucleotide or protein sequence means without deletions or gaps in the sequence.

In addition to the guide sequence, a guide RNA may further comprise one or more other structural or scaffold sequence(s), which may bind or interact with an RNA-guided endonuclease. Such scaffold or structural sequences may further interact with other RNA molecules (e.g., tracrRNA). Methods and techniques for designing targeting constructs and guide RNAs for genome editing and site-directed integration at a target site within the genome of a plant using an RNA-guided endonuclease are known in the art.

According to some embodiments, recombinant DNA molecules, constructs and vectors are provided comprising a polynucleotide or transcribable DNA sequence encoding a site-specific nuclease, such as a zinc-finger nuclease (ZFN), a meganuclease, an RNA-guided endonuclease, a TALE-endonuclease (TALEN), a recombinase, or a transposase, wherein the coding sequence is operably linked to a plant expressible promoter. For RNA-guided endonucleases, recombinant DNA molecules, constructs and vectors are further provided comprising a polynucleotide or transcribable DNA sequence encoding a guide RNA, wherein the guide RNA comprises a guide sequence of sufficient length having a percent identity or complementarity to a target site within the genome of a plant. According to some embodiments, recombinant DNA molecules, constructs and vectors are provided comprising a first polynucleotide or transcribable DNA sequence encoding a site-specific nuclease and a second polynucleotide or transcribable DNA sequence encoding one or more gRNAs. According to some embodiments, each polynucleotide or transcribable DNA sequence of a recombinant DNA molecule, construct and vector that encodes a site-specific nuclease and/or a guide RNA may be operably linked to a plant expressible promoter, such as an inducible promoter, a constitutive promoter, a tissue-specific promoter, etc.

According to some embodiments, a recombinant DNA molecule, construct or vector may comprise a first polynucleotide sequence encoding a site-specific nuclease and a second polynucleotide sequence encoding a guide RNA that may be introduced into a plant cell together via plant transformation techniques. Alternatively, two recombinant DNA molecules, constructs or vectors may be provided including a first recombinant DNA molecule, construct or vector and a second DNA molecule, construct or vector that may be introduced into a plant cell together or sequentially via plant transformation techniques, wherein the first recombinant DNA molecule, construct or vector comprises a polynucleotide sequence encoding a site-specific nuclease and the second recombinant DNA molecule, construct or vector comprises a polynucleotide sequence encoding a guide RNA. According to some embodiments, a recombinant DNA molecule, construct or vector comprising a polynucleotide sequence encoding a site-specific nuclease may be introduced via plant transformation techniques into a plant cell that already comprises (or is transformed with) a recombinant DNA construct or vector comprising a polynucleotide sequence encoding a guide RNA. Alternatively, a recombinant DNA molecule, construct or vector comprising a polynucleotide sequence encoding a guide RNA may be introduced via plant transformation techniques into a plant cell that already comprises (or is transformed with) a recombinant DNA construct or vector comprising a polynucleotide sequence encoding a site-specific nuclease. According to yet further embodiments, a first plant comprising (or transformed with) a recombinant DNA construct or vector comprising a polynucleotide sequence encoding a site-specific nuclease may be crossed with a second plant comprising (or transformed with) a recombinant DNA construct or vector comprising a polynucleotide sequence encoding a guide RNA. Such recombinant DNA molecules, constructs or vectors may be transiently transformed into a plant cell or stably transformed or more preferably integrated into the genome of a plant cell.

In an aspect, molecules or vectors comprising polynucleotides encoding a site-specific nuclease, and optionally one or more, two or more, three or more, or four or more gRNAs are provided to a plant cell by transformation methods known in the art (e.g., without being limiting, particle bombardment, PEG-mediated protoplast transfection or Agrobacterium-mediated transformation). In an aspect, molecules or vectors comprising polynucleotides encoding a Cas9 nuclease, and optionally one or more, two or more, three or more, or four or more gRNAs are provided to a plant cell by transformation methods known in the art (e.g., without being limiting, particle bombardment, PEG-mediated protoplast transfection or Agrobacterium-mediated transformation). In another aspect, vectors comprising polynucleotides encoding a Cpf1 and, optionally one or more, two or more, three or more, or four or more crRNAs are provided to a cell by transformation methods known in the art (e.g., without being limiting, viral transfection, particle bombardment, PEG-mediated protoplast transfection or Agrobacterium-mediated transformation).

Several site-specific nucleases, such as recombinases, zinc finger nucleases (ZFNs), meganucleases, and TALENs, are not RNA-guided and instead rely on their protein structure to determine their target site for causing the DSB or nick, or they are fused, tethered or attached to a DNA-binding protein domain or motif. The protein structure of the site-specific nuclease (or the fused/attached/tethered DNA binding domain) may target the site-specific nuclease to the target site. According to many of these embodiments, non-RNA-guided site-specific nucleases, such as recombinases, zinc finger nucleases (ZFNs), meganucleases, and TALENs, may be designed, engineered and constructed according to known methods to target and bind to a target site in the genome of a plant, to create a DSB or nick at such genomic target site or locus for integration of a recombinant DNA construct, expression cassette or transgene encoding a GA2 oxidase into the genomic target site or locus. For example, an engineered site-specific nuclease, such as a recombinase, zinc finger nuclease (ZFN), meganuclease, or TALEN, may be designed to target and bind to a genomic target site within the genome of a plant to create a DSB or nick at the genomic target site for integration of a recombinant DNA construct, expression cassette or transgene encoding a GA2 oxidase, wherein such recombinant DNA construct, expression cassette or transgene encoding a GA2 oxidase is provided within an insertion sequence of a donor molecule or template.

In an aspect, a targeted genome editing technique described herein may comprise the use of a zinc finger nuclease (ZFN). ZFNs are synthetic proteins consisting of an engineered zinc finger DNA-binding domain fused to a cleavage domain (or a cleavage half-domain), which may be derived from a restriction endonuclease (e.g., FokI). The DNA binding domain may be canonical (C2H2) or non-canonical (e.g., C3H or C4). The DNA-binding domain can comprise one or more zinc fingers (e.g., 2, 3, 4, 5, 6, 7, 8, 9 or more zinc fingers) depending on the target site. Multiple zinc fingers in a DNA-binding domain may be separated by linker sequence(s). ZFNs can be designed to cleave almost any stretch of double-stranded DNA by modification of the zinc finger DNA-binding domain. ZFNs form dimers from monomers composed of a non-specific DNA cleavage domain (e.g., derived from the FokI nuclease) fused to a DNA-binding domain comprising a zinc finger array engineered to bind a target site DNA sequence. The DNA-binding domain of a ZFN may typically be composed of 3-4 (or more) zinc-fingers. The amino acids at positions −1, +2, +3, and +6 relative to the start of the zinc finger α-helix, which contribute to site-specific binding to the target site, can be changed and customized to fit specific target sequences. The other amino acids may form a consensus backbone to generate ZFNs with different sequence specificities. Methods and rules for designing ZFNs for targeting and binding to specific target sequences are known in the art. See, e.g., US Patent App. Nos. 2005/0064474, 2009/0117617, and 2012/0142062, the contents and disclosures of which are incorporated herein by reference. The FokInuclease domain may require dimerization to cleave DNA and therefore two ZFNs with their C-terminal regions are needed to bind opposite DNA strands of the cleavage site (separated by 5-7 bp). The ZFN monomer can cut the target site if the two-ZF-binding sites are palindromic. A ZFN, as used herein, is broad and includes a monomeric ZFN that can cleave double stranded DNA without assistance from another ZFN. The term ZFN may also be used to refer to one or both members of a pair of ZFNs that are engineered to work together to cleave DNA at the same site.

Without being limited by any scientific theory, because the DNA-binding specificities of zinc finger domains can be re-engineered using one of various methods, customized ZFNs can theoretically be constructed to target nearly any target sequence (e.g., at or near a GA oxidase gene in a plant genome). Publicly available methods for engineering zinc finger domains include Context-dependent Assembly (CoDA), Oligomerized Pool Engineering (OPEN), and Modular Assembly. In an aspect, a method and/or composition provided herein comprises one or more, two or more, three or more, four or more, or five or more ZFNs. In another aspect, a ZFN provided herein is capable of generating a targeted DSB or nick. In an aspect, vectors comprising polynucleotides encoding one or more, two or more, three or more, four or more, or five or more ZFNs are provided to a cell by transformation methods known in the art (e.g., without being limiting, viral transfection, particle bombardment, PEG-mediated protoplast transfection, or Agrobacterium-mediated transformation). The ZFNs may be introduced as ZFN proteins, as polynucleotides encoding ZFN proteins, and/or as combinations of proteins and protein-encoding polynucleotides.

In an aspect, a targeted genome editing technique described herein may comprise the use of a meganuclease. Meganucleases, which are commonly identified in microbes, such as the LAGLIDADG family of homing endonucleases, are unique enzymes with high activity and long recognition sequences (>14 bp) resulting in site-specific digestion of target DNA. Engineered versions of naturally occurring meganucleases typically have extended DNA recognition sequences (for example, 14 to 40 bp). According to some embodiments, a meganuclease may comprise a scaffold or base enzyme selected from the group consisting of I-CreI, I-CeuI, I-MsoI, I-SceI, I-AniI, and I-DmoI. The engineering of meganucleases can be more challenging than ZFNs and TALENs because the DNA recognition and cleavage functions of meganucleases are intertwined in a single domain. Specialized methods of mutagenesis and high-throughput screening have been used to create novel meganuclease variants that recognize unique sequences and possess improved nuclease activity. Thus, a meganuclease may be selected or engineered to bind to a genomic target sequence in a plant, such as at or near the genomic locus of a GA oxidase gene. In an aspect, a method and/or composition provided herein comprises one or more, two or more, three or more, four or more, or five or more meganucleases. In another aspect, a meganuclease provided herein is capable of generating a targeted DSB. In an aspect, vectors comprising polynucleotides encoding one or more, two or more, three or more, four or more, or five or more meganucleases are provided to a cell by transformation methods known in the art (e.g., without being limiting, viral transfection, particle bombardment, PEG-mediated protoplast transfection or Agrobacterium-mediated transformation).

In an aspect, a targeted genome editing technique described herein may comprise the use of a transcription activator-like effector nuclease (TALEN). TALENs are artificial restriction enzymes generated by fusing the transcription activator-like effector (TALE) DNA binding domain to a nuclease domain (e.g., FokI). In some aspects, the nuclease is selected from a group consisting of PvuII, MutH, TevI, FokI, AlwI, MlyI, SbfI, SdaI, StsI, CleDORF, Clo051, and Pept071. For FokI nuclease, when each member of a TALEN pair binds to the DNA sites flanking a target site, the FokI monomers dimerize and cause a double-stranded DNA break at the target site. Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity. The term TALEN, as used herein, is broad and includes a monomeric TALEN that can cleave double stranded DNA without assistance from another TALEN. The term TALEN is also refers to one or both members of a pair of TALENs that work together to cleave DNA at the same site.

Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity. PvuII, MutH, and TevI cleavage domains are useful alternatives to FokI and FokI variants for use with TALEs. PvuII functions as a highly specific cleavage domain when coupled to a TALE (see Yank et al. 2013. PLoS One. 8: e82539). MutH is capable of introducing strand-specific nicks in DNA (see Gabsalilow et al. 2013. Nucleic Acids Research. 41: e83). TevI introduces double-stranded breaks in DNA at targeted sites (see Beurdeley et al., 2013. Nature Communications. 4: 1762).

Transcription activator-like effectors (TALEs) can be engineered to bind practically any DNA sequence, such as at or near the genomic locus of a GA oxidase gene in a plant. TALE has a central DNA-binding domain composed of 13-28 repeat monomers of 33-34 amino acids. The amino acids of each monomer are highly conserved, except for hypervariable amino acid residues at positions 12 and 13. The two variable amino acids are called repeat-variable diresidues (RVDs). The amino acid pairs NI, NG, HD, and NN of RVDs preferentially recognize adenine, thymine, cytosine, and guanine/adenine, respectively, and modulation of RVDs can recognize consecutive DNA bases. This simple relationship between amino acid sequence and DNA recognition has allowed for the engineering of specific DNA binding domains by selecting a combination of repeat segments containing the appropriate RVDs. The relationship between amino acid sequence and DNA recognition of the TALE binding domain allows for designable proteins. Software programs such as DNA Works can be used to design TALE constructs. Other methods of designing TALE constructs are known to those of skill in the art. See Doyle et al., Nucleic Acids Research (2012) 40: W117-122.; Cermak et al., Nucleic Acids Research (2011). 39:e82; and tale-nt.cac.cornell.edu/about. In an aspect, a method and/or composition provided herein comprises one or more, two or more, three or more, four or more, or five or more TALENs. In another aspect, a TALEN provided herein is capable of generating a targeted DSB. In an aspect, vectors comprising polynucleotides encoding one or more, two or more, three or more, four or more, or five or more TALENs are provided to a cell by transformation methods known in the art (e.g., without being limiting, viral transfection, particle bombardment, PEG-mediated protoplast transfection or Agrobacterium-mediated transformation). See, e.g., US Patent App. Nos. 2011/0145940, 2011/0301073, and 2013/0117869, the contents and disclosures of which are incorporated herein by reference.

In an aspect, a targeted genome editing technique described herein may comprise the use of a recombinase. In some embodiments, a tyrosine recombinase attached, etc., to a DNA recognition domain or motif may be selected from the group consisting of a Cre recombinase, a Flp recombinase, and a Tnp1 recombinase. In an aspect, a Cre recombinase or a Gin recombinase provided herein may be tethered to a zinc-finger DNA binding domain. The Flp-FRT site-directed recombination system may come from the 2p plasmid from the baker's yeast Saccharomyces cerevisiae. In this system, Flp recombinase (flippase) may recombine sequences between flippase recognition target (FRT) sites. FRT sites comprise 34 nucleotides. Flp may bind to the “arms” of the FRT sites (one arm is in reverse orientation) and cleaves the FRT site at either end of an intervening nucleic acid sequence. After cleavage, Flp may recombine nucleic acid sequences between two FRT sites. Cre-lox is a site-directed recombination system derived from the bacteriophage P1 that is similar to the Flp-FRT recombination system. Cre-lox can be used to invert a nucleic acid sequence, delete a nucleic acid sequence, or translocate a nucleic acid sequence. In this system, Cre recombinase may recombine a pair of lox nucleic acid sequences. Lox sites comprise 34 nucleotides, with the first and last 13 nucleotides (arms) being palindromic. During recombination, Cre recombinase protein binds to two lox sites on different nucleic acids and cleaves at the lox sites. The cleaved nucleic acids are spliced together (reciprocally translocated) and recombination is complete. In another aspect, a lox site provided herein is a loxP, lox 2272, loxN, lox 511, lox 5171, lox71, lox66, M2, M3, M7, or M11 site.

According to another aspect of the present disclosure, a transgenic plant(s), plant cell(s), seed(s), and plant part(s) are provided comprising a transformation event or insertion into the genome of at least one plant cell thereof, wherein the transformation event or insertion comprises a recombinant DNA sequence, construct or expression cassette comprising a transcribable DNA sequence encoding a GA2 oxidase, wherein the transcribable DNA sequence is operably linked to a plant-expressible promoter, such as a constitutive, vascular and/or leaf promoter. Such a transgenic plant may be produced by any suitable transformation method as provided above, to produce a transgenic R₀ plant, which may then be selfed or crossed to other plants to generate R₁ seed and subsequent progeny generations and seed through additional crosses, etc. Embodiments of the present disclosure further include a plant cell, tissue, explant, plant part, etc., comprising one or more transgenic cells having a transformation event or genomic insertion of a recombinant DNA or polynucleotide sequence comprising a transcribable DNA sequence encoding a GA2 oxidase mRNA and protein.

Transgenic plants, plant cells, seeds, and plant parts of the present disclosure may be homozygous or hemizygous for a transgenic event or insertion of an expression cassette or transcribable DNA sequence encoding a GA2 oxidase, and plants, plant cells, seeds, and plant parts of the present embodiments may contain any number of copies of such transgenic event(s) and/or insertion(s). The dosage or amount of expression of a transgene or transcribable DNA sequence may be altered by its copy number and the combination of transgenes or transcribable DNA sequences, which may affect the degree or extent of phenotypic changes in the transgenic plant, etc. Transgenic plants provided herein may corn plants already having increased yield and/or lodging resistance due to prior breeding efforts and mutations of the GA pathway in these plants. Advantages of using a transgene or transcribable DNA sequence to ectopically express a GA2 oxidase gene is not only the ability to limit expression in a tissue-specific or tissue-preferred manner, but also the potential dominance (e.g., dominant negative effects) of a single or hemizygous copy of the transcribable DNA sequence to cause the beneficial short-stature, semi-dwarf traits or phenotypes in crop plants. Thus, recombinant DNA molecules or constructs of the present disclosure may be used to create beneficial traits in corn plants without off-types using only a single copy of the transgenic event, insertion or construct. Unlike previously described mutations or alleles in the GA pathway that are recessive and require plants to be homozygous for the mutant allele, plants transformed with a GA2 oxidase transgene of the present disclosure may improve traits, yield and crop breeding efforts by facilitating the production of hybrid corn plants since they only require a single or hemizygous copy of the transgene.

According to some embodiments, a transgenic or modified corn plant comprising a GA2 oxidase transgene may be characterized as having one or more beneficial traits, such as a shorter stature or semi-dwarf plant height, reduced internode length, increased stalk/stem diameter, improved lodging resistance, reduced green snap, deeper roots, increased leaf area, earlier canopy closure, increased foliar water content and/or higher stomatal conductance under water limiting conditions, reduced anthocyanin content and/or area in leaves under normal or nitrogen or water limiting stress conditions, improved yield-related traits including a larger female reproductive organ or ear, an increase in ear weight, harvest index, yield, seed or kernel number, and/or seed or kernel weight, relative to a wild type or control plant. Such a transgenic corn plant may further have increased stress tolerance, such as increased drought tolerance, nitrogen utilization, and/or tolerance to high density planting.

For purposes of the present disclosure, a “plant” includes an explant, plant part, seedling, plantlet or whole plant at any stage of regeneration or development. As used herein, a “transgenic plant” refers to a plant whose genome has been altered by the integration or insertion of a recombinant DNA molecule, construct or sequence. A transgenic plant includes an R₀ plant developed or regenerated from an originally transformed plant cell(s) as well as progeny transgenic plants in later generations or crosses from the R₀ transgenic plant. As used herein, a “plant part” refers to any organ or intact tissue of a plant, such as a meristem, shoot organ/structure (e.g., leaf, stem or node), root, flower or floral organ/structure (e.g., bract, sepal, petal, stamen, carpel, anther and ovule), seed (e.g., embryo, endosperm, and seed coat), fruit (e.g., the mature ovary), propagule, or other plant tissues (e.g., vascular tissue, dermal tissue, ground tissue, and the like), or any portion thereof. Plant parts of the present disclosure may be viable, nonviable, regenerable, and/or non-regenerable. A “propagule” may include any plant part that can grow into an entire plant.

According to present embodiments, a plant cell transformed with a construct or molecule comprising a transcribable DNA sequence encoding a GA2 oxidase may include any plant cell that is competent for transformation as understood in the art based on the method of transformation, such as a meristem cell, an embryonic cell, a callus cell, etc. As used herein, a “transgenic plant cell” simply refers to any plant cell that is transformed with a stably-integrated recombinant DNA molecule, construct or sequence. A transgenic plant cell may include an originally-transformed plant cell, a transgenic plant cell of a regenerated or developed R₀ plant, a transgenic plant cell cultured from another transgenic plant cell, or a transgenic plant cell from any progeny plant or offspring of the transformed R₀ plant, including cell(s) of a plant seed or embryo, or a cultured plant cell, callus cell, etc.

Methods and techniques are provided for screening for, and/or identifying, cells or plants, etc., for the presence of targeted edits or transgenes, and selecting cells or plants comprising targeted edits or transgenes, which may be based on one or more phenotypes or traits, or on the presence or absence of a molecular marker or polynucleotide or protein sequence in the cells or plants. Embodiments of the present disclosure include methods for making or producing transgenic or modified plants, such as by transformation, site-directed integration, crossing, etc., wherein the method comprises introducing a recombinant DNA molecule, construct or sequence comprising a GA2 oxidase transgene into a plant cell, and then regenerating or developing the transgenic or modified plant from the transformed plant cell, which may be performed under selection pressure favoring a transgenic event. Such methods may comprise transforming a plant cell with a recombinant DNA molecule, construct or sequence comprising the transcribable DNA sequence, and selecting for a plant having one or more altered phenotypes or traits, such as one or more of the following traits at one or more stages of development: shorter or semi-dwarf stature or plant height, shorter internode length in one or more internode(s), increased stalk/stem diameter, improved lodging resistance, reduced green snap, deeper roots, increased leaf area, earlier canopy closure, increased foliar water content and/or higher stomatal conductance under water limiting conditions, reduced anthocyanin content and/or area in leaves under normal or nitrogen or water limiting stress conditions, improved yield-related traits including a larger female reproductive organ or ear, an increase in ear weight, harvest index, yield, seed or kernel number, and/or seed or kernel weight, increased stress tolerance, such as increased drought tolerance, increased nitrogen utilization, and/or increased tolerance to high density planting, as compared to a wild type or control plant. Alternatively or additionally, such methods may comprise transforming a plant cell with a recombinant DNA molecule, construct or sequence comprising the transcribable DNA sequence, and selecting for a plant having the recombinant DNA molecule, construct or sequence according to any molecular biology techniques known in the art.

Nucleic acids can be isolated and detected using techniques known in the art. For example, nucleic acids can be isolated and detected using, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides. Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis. Any method known in the art may be used to screen for, and/or identify, cells, plants, etc., having a transgene or genome edit in its genome, which may be based on any suitable form of visual observation, selection, molecular technique, etc.

In some embodiments, methods are provided for detecting recombinant nucleic acids and/or polypeptides in plant cells. For example, nucleic acids may be detected using hybridization probes or through production of amplicons using PCR with primers as known in the art. Hybridization between nucleic acids is discussed in Sambrook et al. (1989, Molecular Cloning: A Laboratory Manual, 2nd Ed., Cold Spring Harbor Laboratory Press, Cold Spring Harbor, NY). Polypeptides can be detected using antibodies. Techniques for detecting polypeptides using antibodies include enzyme linked immunosorbent assays (ELISAs), Western blots, immunoprecipitations, immunofluorescence, and the like. An antibody provided herein may be a polyclonal antibody or a monoclonal antibody. An antibody having specific binding affinity for a polypeptide provided herein can be generated using methods known in the art. An antibody or hybridization probe may be attached to a solid support, such as a tube, plate or well, using methods known in the art. Detection (e.g., of an amplification product, of a hybridization complex, of a polypeptide) can be accomplished using detectable labels that may be attached or associated with a hybridization probe or antibody. The term “label” is intended to encompass the use of direct labels as well as indirect labels. Detectable labels include enzymes, prosthetic groups, fluorescent materials, luminescent materials, bioluminescent materials, and radioactive materials.

The screening and selection of modified, edited or transgenic plants or plant cells can be through any methodologies known to those skilled in the art of molecular biology. Examples of screening and selection methodologies include, but are not limited to, Southern analysis, PCR amplification for detection of a polynucleotide, Northern blots, RNase protection, primer-extension, RT-PCR amplification for detecting RNA transcripts, Sanger sequencing, Next Generation sequencing technologies (e.g., Illumina®, PacBio®, Ion TorrentTM, etc.) enzymatic assays for detecting enzyme or ribozyme activity of polypeptides and polynucleotides, and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known in the art.

According to another aspect of the present disclosure, methods are provided for planting a modified or transgenic plant(s) provided herein at a normal/standard or high density in field. According to some embodiments, the yield of a crop plant per acre (or per land area) may be increased by planting a modified or transgenic plant(s) of the present disclosure at a higher density in the field. As described herein, modified or transgenic plants expressing a transcribable DNA sequence that encodes a GA2 oxidase may have reduced plant height, shorter internode(s), increased stalk/stem diameter, and/or increased lodging resistance. It is proposed that modified or transgenic plants may tolerate high density planting conditions since an increase in stem diameter may resist lodging and the shorter plant height may allow for increased light penetrance to the lower leaves under high density planting conditions. Thus, modified or transgenic plants provided herein may be planted at a higher density to increase the yield per acre (or land area) in the field. For row crops, higher density may be achieved by planting a greater number of seeds/plants per row length and/or by decreasing the spacing between rows.

According to some embodiments, a modified or transgenic crop plant may be planted at a density in the field (plants per land/field area) that is at least 5%, 10%, 15%, 20%, 25%, 50%, 75%, 100%, 125%, 150%, 175%, 200%, 225%, or 250% higher than the normal planting density for that crop plant according to standard agronomic practices. A modified or transgenic crop plant may be planted at a density in the field of at least 38,000 plants per acre, at least 40,000 plants per acre, at least 42,000 plants per acre, at least 44,000 plants per acre, at least 45,000 plants per acre, at least 46,000 plants per acre, at least 48,000 plants per acre, 50,000 plants per acre, at least 52,000 plants per acre, at least 54,000 per acre, or at least 56,000 plants per acre. As an example, corn plants may be planted at a higher density, such as in a range from about 38,000 plants per acre to about 60,000 plants per acre, or about 40,000 plants per acre to about 58,000 plants per acre, or about 42,000 plants per acre to about 58,000 plants per acre, or about 40,000 plants per acre to about 45,000 plants per acre, or about 45,000 plants per acre to about 50,000 plants per acre, or about 50,000 plants per acre to about 58,000 plants per acre, or about 52,000 plants per acre to about 56,000 plants per acre, or about 38,000 plants per acre, about 42,000 plant per acre, about 46,000 plant per acre, or about 48,000 plants per acre, about 50,000 plants per acre, or about 52,000 plants per acre, or about 54,000 plant per acre, as opposed to a standard density range, such as about 18,000 plants per acre to about 38,000 plants per acre.

According to embodiments of the present disclosure, a modified corn plant(s) is/are provided that comprise (i) a plant height of less than 2000 mm, less than 1950 mm, less than 1900 mm, less than 1850 mm, less than 1800 mm, less than 1750 mm, less than 1700 mm, less than 1650 mm, less than 1600 mm, less than 1550 mm, less than 1500 mm, less than 1450 mm, less than 1400 mm, less than 1350 mm, less than 1300 mm, less than 1250 mm, less than 1200 mm, less than 1150 mm, less than 1100 mm, less than 1050 mm, or less than 1000 mm, and/or (ii) an average stem or stalk diameter of at least 18 mm, at least 18.5 mm, at least 19 mm, at least 19.5 mm, at least 20 mm, at least 20.5 mm, at least 21 mm, at least 21.5 mm, or at least 22 mm. Stated a different way, a modified corn plant(s) is/are provided that comprise a plant height of less than 2000 mm, less than 1950 mm, less than 1900 mm, less than 1850 mm, less than 1800 mm, less than 1750 mm, less than 1700 mm, less than 1650 mm, less than 1600 mm, less than 1550 mm, less than 1500 mm, less than 1450 mm, less than 1400 mm, less than 1350 mm, less than 1300 mm, less than 1250 mm, less than 1200 mm, less than 1150 mm, less than 1100 mm, less than 1050 mm, or less than 1000 mm, and/or an average stem or stalk diameter that is greater than 18 mm, greater than 18.5 mm, greater than 19 mm, greater than 19.5 mm, greater than 20 mm, greater than 20.5 mm, greater than 21 mm, greater than 21.5 mm, or greater than 22 mm. Any such plant height trait or range that is expressed in millimeters (mm) may be converted into a different unit of measurement based on known conversions (e.g., one inch is equal to 2.54 cm or 25.4 millimeters, and millimeters (mm), centimeters (cm) and meters (m) only differ by one or more powers of ten). Thus, any measurement provided herein is further described in terms of any other comparable units of measurement according to known and established conversions. However, the exact plant height and/or stem diameter of a modified corn plant may depend on the environment and genetic background. Thus, the change in plant height and/or stem diameter of a modified corn plant may instead be described in terms of a minimum difference or percent change relative to a control plant. A modified corn plant may further comprise at least one ear that is substantially free of male reproductive tissues or structures or other off-types.

According to embodiments of the present disclosure, modified corn plants are provided that comprise a plant height during late vegetative and/or reproductive stages of development (e.g., at R3 stage) of between 1000 mm and 1800 mm, between 1000 mm and 1700 mm, between 1050 mm and 1700 mm, between 1100 mm and 1700 mm, between 1150 mm and 1700 mm, between 1200 mm and 1700 mm, between 1250 mm and 1700 mm, between 1300 mm and 1700 mm, between 1350 mm and 1700 mm, between 1400 mm and 1700 mm, between 1450 mm and 1700 mm, between 1000 mm and 1500 mm, between 1050 mm and 1500 mm, between 1100 mm and 1500 mm, between 1150 mm and 1500 mm, between 1200 mm and 1500 mm, between 1250 mm and 1500 mm, between 1300 mm and 1500 mm, between 1350 mm and 1500 mm, between 1400 mm and 1500 mm, between 1450 mm and 1500 mm, between 1000 mm and 1600 mm, between 1100 mm and 1600 mm, between 1200 mm and 1600 mm, between 1300 mm and 1600 mm, between 1350 mm and 1600 mm, between 1400 mm and 1600 mm, between 1450 mm and 1600 mm, of between 1000 mm and 2000 mm, between 1200 mm and 2000 mm, between 1200 mm and 1800 mm, between 1300 mm and 1700 mm, between 1400 mm and 1700 mm, between 1400 mm and 1600 mm, between 1400 mm and 1700 mm, between 1400 mm and 1800 mm, between 1400 mm and 1900 mm, between 1400 mm and 2000 mm, or between 1200 mm and 2500 mm, and/or an average stem diameter of between 17.5 mm and 22 mm, between 18 mm and 22 mm, between 18.5 and 22 mm, between 19 mm and 22 mm, between 19.5 mm and 22 mm, between 20 mm and 22 mm, between 20.5 mm and 22 mm, between 21 mm and 22 mm, between 21.5 mm and 22 mm, between 17.5 mm and 21 mm, between 17.5 mm and 20 mm, between 17.5 mm and 19 mm, between 17.5 mm and 18 mm, between 18 mm and 21 mm, between 18 mm and 20 mm, or between 18 mm and 19 mm. A modified corn plant may be substantially free of off-types, such as male reproductive tissues or structures in one or more ears of the modified corn plant.

According to embodiments of the present disclosure, modified corn plants are provided that have (i) a plant height that is at least 5%, at least 10%, at least 15%, at least 20%, at least 25%, at least 30%, at least 35%, at least 40%, at least 45%, at least 50%, at least 55%, at least 60%, at least 65%, at least 70%, or at least 75% less than the height of a wild-type or control plant, and/or (ii) a stem or stalk diameter that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% greater than the stem diameter of the wild-type or control plant. According to embodiments of the present disclosure, a modified corn plant may have a reduced plant height that is no more than 20%, 25%, 30%, 35%, 40%, 45%, 50%, 55%, or 60% shorter than the height of a wild-type or control plant, and/or a stem or stalk diameter that is less than (or not more than) 10%, 15%, 20%, 25%, 30%, 35%, 40%, 45%, 50%, 55%, 60%, 65%, 70%, 75%, 80%, 85%, 90%, 95%, or 100% greater than the stem or stalk diameter of a wild-type or control plant. For example, a modified plant may have (i) a plant height that is at least 10%, at least 15%, or at least 20% less or shorter (i.e., greater than or equal to 10%, 15%, or 20% shorter), but not greater or more than 50% shorter, than a wild type or control plant, and/or (ii) a stem or stalk diameter that is that is at least 5%, at least 10%, or at least 15% greater, but not more than 30%, 35%, or 40% greater, than a wild type or control plant. For clarity, the phrases “at least 20% shorter” and “greater than or equal to 20% shorter” would exclude, for example, 10% shorter. Likewise for clarity, the phrases “not greater than 50% shorter”, “no more than 50% shorter” and “not more than 50% shorter” would exclude 60% shorter; the phrase “at least 5% greater” would exclude 2% greater; and the phrases “not more than 30% greater” and “no more than 30% greater” would exclude 40% greater.

According to embodiments of the present disclosure, modified corn plants are provided that comprise a height between 5% and 75%, between 5% and 50%, between 10% and 70%, between 10% and 65%, between 10% and 60%, between 10% and 55%, between 10% and 50%, between 10% and 45%, between 10% and 40%, between 10% and 35%, between 10% and 30%, between 10% and 25%, between 10% and 20%, between 10% and 15%, between 10% and 10%, between 10% and 75%, between 25% and 75%, between 10% and 50%, between 20% and 50%, between 25% and 50%, between 30% and 75%, between 30% and 50%, between 25% and 50%, between 15% and 50%, between 20% and 50%, between 25% and 45%, or between 30% and 45% less than the height of a wild-type or control plant, and/or a stem or stalk diameter that is between 5% and 100%, between 5% and 95%, between 5% and 90%, between 5% and 85%, between 5% and 80%, between 5% and 75%, between 5% and 70%, between 5% and 65%, between 5% and 60%, between 5% and 55%, between 5% and 50%, between 5% and 45%, between 5% and 40%, between 5% and 35%, between 5% and 30%, between 5% and 25%, between 5% and 20%, between 5% and 15%, between 5% and 10%, between 10% and 100%, between 10% and 75%, between 10% and 50%, between 10% and 40%, between 10% and 30%, between 10% and 20%, between 25% and 75%, between 25% and 50%, between 50% and 75%, between 8% and 20%, or between 8% and 15% greater than the stem or stalk diameter of the wild-type or control plant.

According to embodiments of the present disclosure, modified corn plants are provided that comprise an average internode length (or a minus-2 internode length and/or minus-4 internode length relative to the position of the ear) that is at least 5%, at least 10%, at least 15%, at least 20%, at least 25%, at least 30%, at least 35%, at least 40%, at least 45%, at least 50%, at least 55%, at least 60%, at least 65%, at least 70%, or at least 75% less than the same or average internode length of a wild-type or control plant. The “minus-2 internode” of a corn plant refers to the second internode below the ear of the plant, and the “minus-4 internode” of a corn plant refers to the fourth internode below the ear of the plant According to many embodiments, modified corn plants are provided that have an average internode length (or a minus-2 internode length and/or minus-4 internode length relative to the position of the ear) that is between 5% and 75%, between 5% and 50%, between 10% and 70%, between 10% and 65%, between 10% and 60%, between 10% and 55%, between 10% and 50%, between 10% and 45%, between 10% and 40%, between 10% and 35%, between 10% and 30%, between 10% and 25%, between 10% and 20%, between 10% and 15%, between 10% and 10%, between 10% and 75%, between 25% and 75%, between 10% and 50%, between 20% and 50%, between 25% and 50%, between 30% and 75%, between 30% and 50%, between 25% and 50%, between 15% and 50%, between 20% and 50%, between 25% and 45%, or between 30% and 45% less than the same or average internode length of a wild-type or control plant.

According to embodiments of the present disclosure, modified corn plants are provided that comprise an ear weight (individually or on average) that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% greater than the ear weight of a wild-type or control plant. A modified corn plant provided herein may comprise an ear weight that is between 5% and 100%, between 5% and 95%, between 5% and 90%, between 5% and 85%, between 5% and 80%, between 5% and 75%, between 5% and 70%, between 5% and 65%, between 5% and 60%, between 5% and 55%, between 5% and 50%, between 5% and 45%, between 5% and 40%, between 5% and 35%, between 5% and 30%, between 5% and 25%, between 5% and 20%, between 5% and 15%, between 5% and 10%, between 10% and 100%, between 10% and 75%, between 10% and 50%, between 25% and 75%, between 25% and 50%, or between 50% and 75% greater than the ear weight of a wild-type or control plant.

According to embodiments of the present disclosure, modified corn plants are provided that have a harvest index of at least 0.57, at least 0.58, at least 0.59, at least 0.60, at least 0.61, at least 0.62, at least 0.63, at least 0.64, or at least 0.65 (or greater). A modified corn plant may comprise a harvest index of between 0.57 and 0.65, between 0.57 and 0.64, between 0.57 and 0.63, between 0.57 and 0.62, between 0.57 and 0.61, between 0.57 and 0.60, between 0.57 and 0.59, between 0.57 and 0.58, between 0.58 and 0.65, between 0.59 and 0.65, or between 0.60 and 0.65. A modified corn plant may have a harvest index that is at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, or at least 50% greater than the harvest index of a wild-type or control plant. A modified corn plant may have a harvest index that is between 1% and 45%, between 1% and 40%, between 1% and 35%, between 1% and 30%, between 1% and 25%, between 1% and 20%, between 1% and 15%, between 1% and 14%, between 1% and 13%, between 1% and 12%, between 1% and 11%, between 1% and 10%, between 1% and 9%, between 1% and 8%, between 1% and 7%, between 1% and 6%, between 1% and 5%, between 1% and 4%, between 1% and 3%, between 1% and 2%, between 5% and 15%, between 5% and 20%, between 5% and 30%, or between 5% and 40% greater than the harvest index of a wild-type or control plant.

According to embodiments of the present disclosure, modified corn plants are provided that have an increase in harvestable yield of at least 1 bushel per acre, at least 2 bushels per acre, at least 3 bushels per acre, at least 4 bushels per acre, at least 5 bushels per acre, at least 6 bushels per acre, at least 7 bushels per acre, at least 8 bushels per acre, at least 9 bushels per acre, or at least 10 bushels per acre, relative to a wild-type or control plant. A modified corn plant may have an increase in harvestable yield between 1 and 10, between 1 and 8, between 2 and 8, between 2 and 6, between 2 and 5, between 2.5 and 4.5, or between 3 and 4 bushels per acre. A modified corn plant may have an increase in harvestable yield that is at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, or at least 25% greater than the harvestable yield of a wild-type or control plant. A modified corn plant may have a harvestable yield that is between 1% and 25%, between 1% and 20%, between 1% and 15%, between 1% and 14%, between 1% and 13%, between 1% and 12%, between 1% and 11%, between 1% and 10%, between 1% and 9%, between 1% and 8%, between 1% and 7%, between 1% and 6%, between 1% and 5%, between 1% and 4%, between 1% and 3%, between 1% and 2%, between 5% and 15%, between 5% and 20%, between 5% and 25%, between 2% and 10%, between 2% and 9%, between 2% and 8%, between 2% and 7%, between 2% and 6%, between 2% and 5%, or between 2% and 4% greater than the harvestable yield of a wild-type or control plant.

According to embodiments of the present disclosure, a modified corn plant is provided that has a lodging frequency that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% less or lower than a wild-type or control plant. A modified corn plant may have a lodging frequency that is between 5% and 100%, between 5% and 95%, between 5% and 90%, between 5% and 85%, between 5% and 80%, between 5% and 75%, between 5% and 70%, between 5% and 65%, between 5% and 60%, between 5% and 55%, between 5% and 50%, between 5% and 45%, between 5% and 40%, between 5% and 35%, between 5% and 30%, between 5% and 25%, between 5% and 20%, between 5% and 15%, between 5% and 10%, between 10% and 100%, between 10% and 75%, between 10% and 50%, between 10% and 40%, between 10% and 30%, between 10% and 20%, between 25% and 75%, between 25% and 50%, or between 50% and 75% less or lower than a wild-type or control plant. Further provided are populations of corn plants having increased lodging resistance and a reduced lodging frequency. Populations of modified corn plants are provided having a lodging frequency that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% less or lower than a population of wild-type or control plants. A population of modified corn plants may comprise a lodging frequency that is between 5% and 100%, between 5% and 95%, between 5% and 90%, between 5% and 85%, between 5% and 80%, between 5% and 75%, between 5% and 70%, between 5% and 65%, between 5% and 60%, between 5% and 55%, between 5% and 50%, between 5% and 45%, between 5% and 40%, between 5% and 35%, between 5% and 30%, between 5% and 25%, between 5% and 20%, between 5% and 15%, between 5% and 10%, between 10% and 100%, between 10% and 75%, between 10% and 50%, between 10% and 40%, between 10% and 30%, between 10% and 20%, between 25% and 75%, between 25% and 50%, or between 50% and 75% less or lower than a population of wild-type or control plants, which may be expressed as an average over a specified number of plants or crop area of equal density.

According to embodiments of the present disclosure, modified corn plants are provided having a significantly reduced or decreased plant height (e.g., 2000 mm or less) and/or a significantly increased stem diameter (e.g., 18 mm or more), relative to a wild-type or control plant. According to these embodiments, the decrease or reduction in plant height and/or increase in stem diameter may be within any of the height, diameter or percentage ranges recited herein. Such modified corn plants having a reduced plant height and/or increased stem diameter relative to a wild-type or control plant may be transformed with a transcribable DNA sequence encoding a GA2 oxidase mRNA and protein. Modified corn plants having a significantly reduced plant height and/or a significantly increased stem diameter relative to a wild-type or control plant may further have at least one ear that is substantially free of male reproductive tissues or structures and/or other off-types. Modified corn plants having a significantly reduced plant height and/or an increased stem diameter relative to a wild-type or control plant may have ectopic expression of a GA2 oxidase mRNA and/or protein in one or more tissue(s) of the plant, such as one or more vascular and/or leaf tissue(s) of the plant, relative to the same tissue(s) of the wild-type or control plant. According to many embodiments, modified corn plants may comprise at least one polynucleotide or transcribable DNA sequence encoding a GA2 oxidase operably linked to a plant-expressible promoter as provided herein, which may be a constitutive, tissue-specific or tissue-preferred promoter. According to some embodiments, modified corn plants having a significantly reduced plant height and/or an increased stem diameter relative to a wild-type or control plant may further have an increased harvest index and/or increased lodging resistance relative to the wild-type or control plant. Such modified corn plants may be substantially free of off-types, such as male reproductive tissues or structures and/or other off-types in at least one ear of the modified plants.

According to embodiments of the present disclosure, a population of modified corn plants are provided, wherein the population of modified corn plants have an average plant height that is significantly less, and/or an average stem or stalk diameter that is significantly more, than a population of wild-type or control plants. The population of modified corn plants may share ancestry with a single modified corn plant and/or have a single transgenic GA2 oxidase construct insertion or event in common. Modified corn plants within a population of modified corn plants may generally comprise at least one ear that is substantially free of male reproductive tissues or structures and/or other off-types. A population of modified corn plants may have increased lodging resistance on average or per number of plants or field area than a population of wild-type or control plants. The population of modified corn plants may have a lodging frequency that is at least 5%, at least 10%, at least 20%, at least 30%, at least 40%, at least 50%, at least 60%, at least 70% at least 80%, at least 90%, or 100% less (or lower) than a population of control corn plants. A population of modified corn plants may have a harvest index of at least 0.57 or greater.

According to embodiments of the present invention, modified corn plants are provided having a reduced gibberellin content (in active form) in at least the stem and internode and possibly other tissue(s), such as the stem, internode, leaf and/or vascular tissue(s), as compared to the same tissue(s) of wild-type or control plants. According to many embodiments, modified corn plants are provided having a significantly reduced plant height and/or a significantly increased stem diameter relative to wild-type or control plants, wherein the modified corn plants further have significantly reduced or decreased level(s) of active gibberellins or active GAs (e.g., one or more of GA1, GA3, GA4, and/or GA7) in one or more stem, internode, leaf and/or vascular tissue(s), relative to the same tissue(s) of the wild-type or control plants. For example, the level of one or more active GAs in the stem, internode, leaf and/or vascular tissue(s) of a modified corn plant may be at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% less or lower than in the same tissue(s) of a wild-type or control corn plant.

According to some embodiments, a modified corn plant may comprise an active gibberellin (GA) level(s) (e.g., one or more of GA1, GA3, GA4, and/or GA7) in one or more stem, internode, leaf and/or vascular tissue(s) that is between 5% and 50%, between 10% and 100%, between 20% and 100%, between 30% and 100%, between 40% and 100%, between 50% and 100%, between 60% and 100%, between 70% and 100%, between 80% and 100%, between 80% and 90%, between 10% and 90%, between 10% and 80%, between 10% and 70%, between 10% and 60%, between 10% and 50%, between 10% and 40%, between 10% and 30%, between 10% and 20%, between 50% and 100%, between 20% and 90%, between 20% and 80%, between 20% and 70%, between 20% and 60%, between 20% and 50%, between 20% and 40%, between 20% and 40%, between 20% and 30%, between 30% and 90%, between 30% and 80%, between 30% and 70%, between 30% and 60%, between 30% and 50%, between 30% and 40%, between 40% and 90% between 40% and 80%, between 40% and 70%, between 40% and 60%, between 40% and 50%, between 50% and 90%, between 50% and 80%, between 50% and 70%, between 50% and 60%, between 60% and 90%, between 60% and 80%, between 60% and 70%, between 70% and 90%, or between 70% and 80% less or (or lower) than in the same tissue(s) of a wild-type or control corn plant. A modified corn plant having a reduced active gibberellin (GA) level(s) in one or more stem, internode, leaf and/or vascular tissue(s) may further be substantially free of off-types, such as male reproductive tissues or structures and/or other off-types in at least one ear of a modified corn plant.

According to many embodiments, a modified corn plant is provided comprising a significantly reduced plant height and/or a significantly increased stem diameter relative to wild-type or control plants, wherein the modified corn plant has transgenic or ectopic GA2 oxidase transcript and protein expression in one or more tissues, such as one or more stem, internode, leaf and/or vascular tissue(s), of the modified plant, as compared to the same tissue(s) of a wild-type or control corn plant. For a transgenic or ectopically expressed GA2 oxidase transcript and/or protein that is identical to an endogenous GA2 oxidase transcript and/or protein in corn, the total expression level of endogenous and transgenic GA2 oxidase transcript and/or protein in one or more stem, internode, leaf and/or vascular tissue(s) of a modified corn plant may be at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% greater than the endogenous GA2 oxidase transcript and/or protein levels in the same tissue(s) of a wild-type or control corn plant.

According to embodiments of the present disclosure, a modified or transgenic corn plant or plant part, one or more modified or transgenic corn plants or plant parts or a plurality modified or transgenic corn plants or plant parts as provided herein, or an agricultural field or soil in which a modified or transgenic corn plant or plant part, one or more modified or transgenic corn plants or plant parts or a plurality modified or transgenic corn plants or plant parts as provided herein are planted or grown, can be treated with an agricultural composition comprising one or more active ingredients or other agents, such as, for example and without limitation, an herbicide or one or more herbicides, a fungicide or one or more fungicides, an insecticide or one or more insecticides, a plant growth regulator or plant stimulant or one or more plant growth regulators and/or plant stimulants, and/or a safener or one or more safeners. Provided below are lists of possible or representative compounds for each of these types of actives or agents, and an agricultural composition may comprise one or any combination or multiplicity of these actives, agents or compounds. Such an agricultural composition may be applied, for example, as a foliar, soil or in-furrow treatment, as a pre-emergent, pre-sowing and/or post-emergent treatment, and/or in some cases, may be applied to modified or transgenic plant part or seed provided herein.

An agricultural composition may be formulated according to its intended use and application. The appropriate formulation of the agricultural composition may be chosen to have different physicochemical parameters, components and stabilities of the respective compound(s). Possible types of formulations for an agricultural composition can include, for example: wettable powders (WP), water-soluble powders(SP), water-soluble concentrates, emulsifiable concentrates (EC), emulsions (EW), such as oil-in-water and water-in-oil emulsions, sprayable solutions, suspension concentrates (SC), dispersions based on oil or water, oil-miscible solutions, capsule suspensions (CS), dusting products (DP), dressings, granules for scattering and soil application, granules (GR) in the form of microgranules, spray granules, absorption and adsorption granules, water-dispersible granules (WG), water-soluble granules (SG), ULV formulations, microcapsules and waxes. If appropriate, some agricultural compositions of a pesticidal compound or one or more pesticidal compounds might be formulated and used as a seed coating applied to a plant part or seed as provided herein.

### EXAMPLES

### Example 1. Corn Plants with Transgenic Expression of Zm.GA2Ox Genes

Three different transformation vectors were created each comprising a recombinant DNA construct comprising a transcribable DNA sequence encoding a different GA2 oxidase operably linked to a RTBV vascular promoter. These transformation vectors included a transcribable DNA sequence encoding one of three GA2 oxidase genes from corn: Zm.GA2 oxidase_2 (nucleotide coding and protein sequences being SEQ ID NOs: 3 and 4, respectively), Zm.GA2 oxidase_3 (nucleotide coding and protein sequences being SEQ ID NOs: 5 and 6, respectively), and Zm.GA2 oxidase_9 (nucleotide coding and protein sequences being SEQ ID NOs: 17 and 18, respectively). Each transformation vector having an expression construct comprising the respective GA2 oxidase coding sequence under the control of a rice tungro bacilliform virus (RTBV) promoter (P-RTBV.1, SEQ ID NO: 333) that is known to cause expression in vascular tissues of plants. For each of the three transformation vectors and constructs, an inbred corn plant line was transformed via Agrobacterium mediated transformation according to known techniques. Several transformation events were generated with each of the three constructs, and these transformants were tested in the greenhouse to determine if they had reduced plant height relative to non-transgenic wild type control plants. Ectopic expression of a GA2 oxidase transgene is expected to reduce the level(s) of bioactive GAs and consequently reduce plant height in corn.

### Example 2. Reduced Plant Height in Inbred Corn Plants with Expression of Various GA2 Oxidase Transgenes

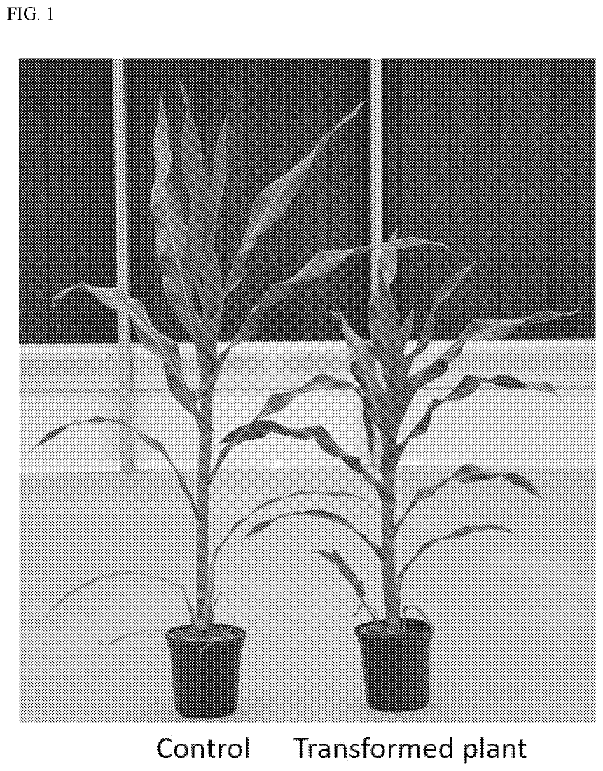

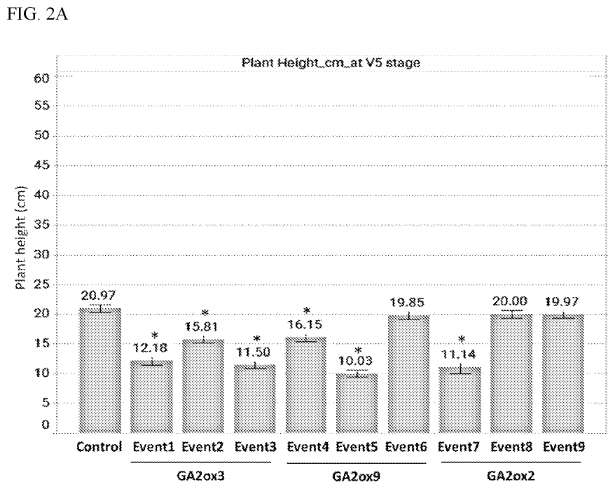

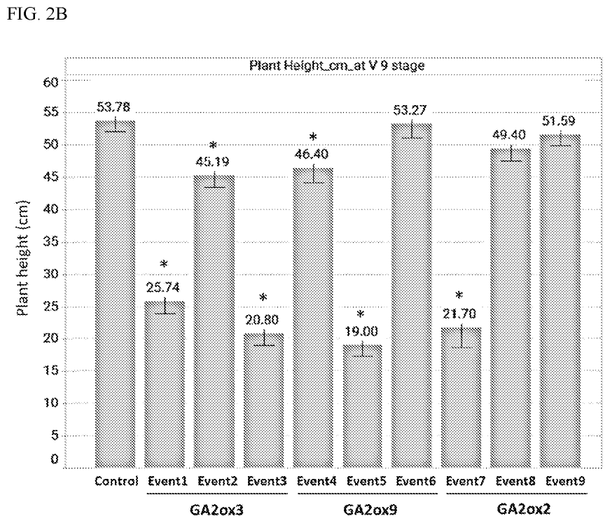

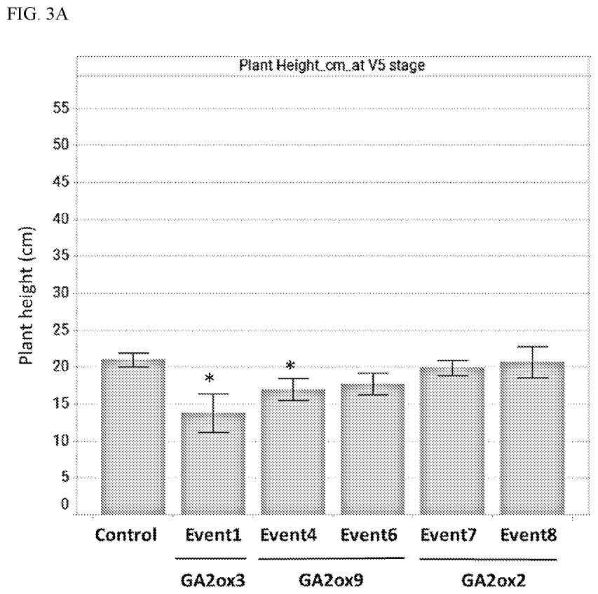

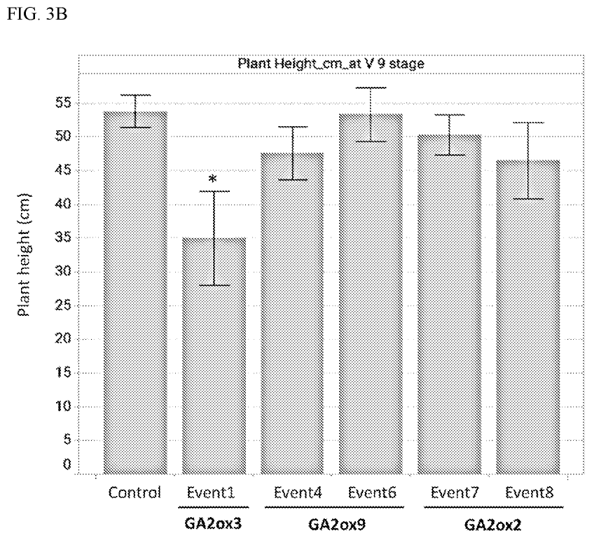

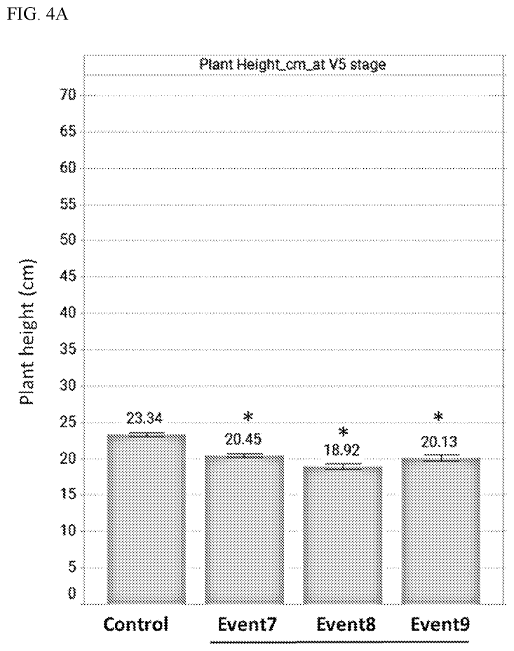

The transformed plants as described in Example 1 were self-crossed to generate inbred plants and tested for zygosity of the transgenic insert. Both homozygous and hemizygous plants were grown in the greenhouse conditions, along with wild type control plants without the transformed event. FIG. 1 shows a representative image of a plant transformed as Event 2 of the GA2ox3 transgene next to a wild type control plant. Plant height (PHT) was measured from soil line to base of highest collared leaf at V5 and V9 vegetative growth stages. A V5 or V9 stage corn plant is defined respectively as a plant with 5 or 9 developed (fully unfolded) leaves of visible leaf collars. Plant height for plants of each of the transformation events was calculated as an average among approximately 10 plants for each event and compared to the average height for control plants. Standard errors were calculated for each event and the control plants, which are represented as error bars in FIG. 2 and FIG. 3. Asterisks (*) indicate a statistical significance between control plants and transgenic plants at p<0.05. As can be seen in Table 18 and FIG. 2, a significant reduction in plant height was observed in two developmental stages in homozygous transgenic plants for one or more events expressing each of three transgenic constructs relative to wild type (WT) control plants. However, for the hemizygous plants in Table 19 and FIG. 3, plants of fewer transformation events had significant plant height reduction, which may be due to reduced expression of the constructs in hemizygous plants.

TABLE 18
Plant Height (in cm) of homozygous inbred corn plants vs. control.

| | | Mean | StdErr | Mean | StdErr |
|---|---|---|---|---|---|
| Gene | Event | (V5) | (V5) | (V9) | (V9) |
| | Control | 21.0 | 0.6 | 53.8 | 1.7 |
| GA2ox3 | Event1 | 12.2 | 0.7 | 25.7 | 1.9 |
| | Event2 | 15.8 | 0.6 | 45.2 | 1.7 |
| | Event3 | 11.5 | 0.6 | 20.8 | 1.8 |
| GA2ox9 | Event4 | 16.2 | 0.8 | 46.4 | 2.2 |
| | Event5 | 10.0 | 0.6 | 19.0 | 1.7 |
| | Event6 | 19.9 | 0.8 | 53.3 | 2.2 |
| GA2ox2 | Event7 | 11.1 | 1.1 | 21.7 | 3.1 |
| | Event8 | 20.0 | 0.7 | 49.4 | 1.9 |
| | Event9 | 20.0 | 0.6 | 51.6 | 1.7 |

TABLE 19
Plant Height (in cm) of hemizygous inbred corn plants vs. control.

| | | Mean | StdErr | Mean | StdErr |
|---|---|---|---|---|---|
| Gene | Event | (V5) | (V5) | (V9) | (V9) |
| | Control | 21.0 | 0.9 | 53.8 | 2.4 |
| GA2ox3 | Event1 | 13.8 | 2.5 | 35.0 | 6.9 |
| GA2ox9 | Event4 | 17.0 | 1.5 | 47.6 | 4.0 |
| | Event6 | 17.8 | 1.5 | 53.3 | 4.0 |
| GA2ox2 | Event7 | 19.9 | 1.1 | 50.3 | 2.9 |
| | Event8 | 20.7 | 2.1 | 46.5 | 5.6 |

This data indicates that transgenic overexpression of either ZmGA2ox2, ZmGA2ox3 or ZmGA2ox9 using the RTBV vascular promoter is effective at causing reduced plant height phenotypes for at least one or more transgenic events. Thus, overexpression of the GA2 oxidase constructs are effective at reducing plant height.

### Example 3. Reduced Plant Height in Hybrid Corn Plants with Expression of ZmGA2ox2 Transgenes

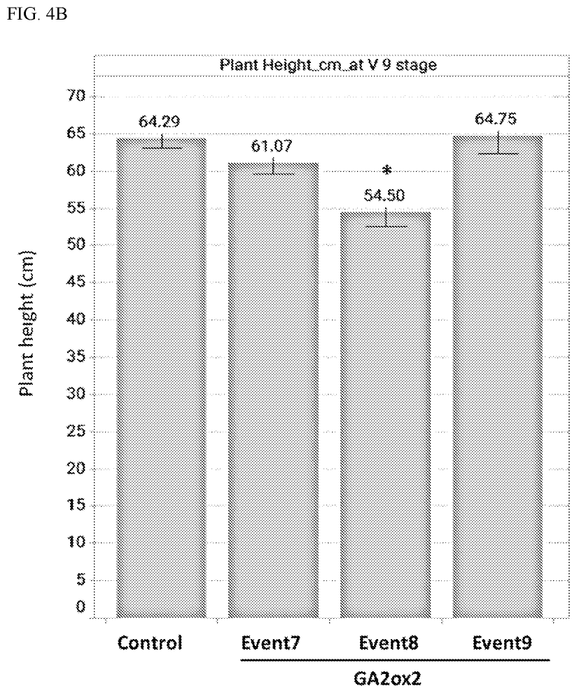

Hybrid corn plants hemizygous for the ZmGA2ox2 events described in Example 1 also showed reduced plant height relative to wild type control plants. Transformed inbred plants homozygous for the respective transgenic event from Example 1 were crossed with another elite parental corn line to generate the hybrid corn plants. These hybrid plants were grown under greenhouse conditions along with wild type control plants. Plant height (PHT) was measured from soil line to base of highest collared leaf at V5 and V9 vegetative growth stages. Plant heights for plants having each of the transformation events for ZmGA2ox2 (Events 7, 8, and 9) were calculated as an average among approximately 10 plants for each event and compared to the average height for wild type control plants. Standard errors were calculated for each event and the control plants, which are represented as error bars in FIG. 4. Standard errors were calculated for the transgenic hybrid and control plants, which are represented as error bars in FIG. 4. Asterisks (*) indicate the statistical significance between control plants and transgenic plants at p<0.05.

As can be seen in Table 20 and FIG. 4, a significant reduction in average plant height was observed in transgenic hybrid plants expressing the GA2ox2 transgene construct, relative to wild type hybrid corn plants, although in this experiment the reduction in plant height was significant for only one event (Event 8) at V9 stage.

TABLE 20
Plant Height (in cm) of hybrid corn plants vs. control.

| | | Mean | StdErr | Mean | StdErr |
|---|---|---|---|---|---|
| Gene | Event | (V5) | (V5) | (V9) | (V9) |
| | Control | 23.3 | 0.2 | 64.3 | 1.2 |
| GA2ox2 | Event7 | 20.5 | 0.3 | 61.1 | 1.5 |
| | Event8 | 18.9 | 0.4 | 54.5 | 1.9 |
| | Event9 | 20.1 | 0.5 | 64.8 | 2.4 |

This data shows that a reduced plant height phenotype is present in hybrid corn plants in addition to inbred lines for at last one or more events per transgenic construct, suggesting semi-dominant nature of the transgene overexpression.

Having described the present disclosure in detail, it will be apparent to those skilled in the art that modifications, variations, and equivalent embodiments are possible without departing from the spirit and scope of the present disclosure as described herein and in the appended claims. Furthermore, it should be appreciated that all examples in the present disclosure are provided as non-limiting examples.

1. A recombinant DNA construct comprising a transcribable DNA sequence encoding a GA2 oxidase protein and a plant-expressible promoter, wherein the transcribable DNA sequence is operably linked to the plant-expressible promoter.

2. The recombinant DNA construct of claim 1, wherein the GA2 oxidase protein is, or comprises an amino acid sequence that is, at least 80%, at least 85%, at least 90%, at least 95%, at least 96%, at least 97%, at least 98%, at least 99%, at least 99.5%, or 100% identical to one or more of SEQ ID NOs: 2, 4, 6, 8, 10, 12, 14, 16, 18, 20, 22, 24, 26; 28, 30, 32, 34, 36, 38, 40, 42, 44, 46; 48, 50, 52, 54, 56, 58, 60, 62; 64, 66, 68, 70, 72, 74, 76, 78, 80, 82, 84, 86, 88, 90, 92, 94; 96, 98, 100, 102, 104, 106, 108, 110, 112, 114, 116, 118, 120, 122, 124; 126, 128, 130, 132, 134, 136, 138, 140, 142, 144, 146; 148, 150, 152, 154, 156, 158, 160, 162, 164, 166, 168, 170, 172, 174, 176, 178; 180, 182, 184, 186, 188, 190, 192, 194, 196, 198, 200, 202, 204, 206, 208; 210, 212, 214, 216, 218, 220, 222, 224, 226, 228, 230, 232, 234, 236, 238; 240, 242, 244, 246, 248, 250, 252; 254, 256, 258, 260, 262, 264, 266; 268, 270, 272, 274, 276, 278, 280, 282, 284; 286, 288, 290, 292; 294, 296, 298, 300, 302, 304, 306, 308; 310, 312, 314, 316, 318, 320, 322; 324, 326; 328, 330 and/or 332.

3. The recombinant DNA construct of claim 1, wherein the transcribable DNA sequence is, or comprises a sequence that is, at least 80%, at least 85%, at least 90%, at least 95%, at least 96%, at least 97%, at least 98%, at least 99%, at least 99.5%, or 100% identical to one or more of SEQ ID NOs: 1, 3, 5, 7, 9, 11, 13, 15, 17, 19, 21, 23 25; 27, 29, 31, 33, 35, 37, 39, 41, 43, 45; 47, 49, 51, 53, 55, 57, 59, 61; 63, 65, 67, 69, 71, 73, 75, 77, 79, 81, 83, 85, 87, 89, 91, 93; 95, 97, 99, 101, 103, 105, 107, 109, 111, 113, 115, 117, 119, 121, 123; 125, 127, 129, 131, 133, 135, 137, 139, 141, 143, 145; 147, 149, 151, 153, 155, 157, 159, 161, 163, 165, 167, 169, 171, 173, 175, 177; 179, 181, 183, 185, 187, 189, 191, 193, 195, 197, 199, 201, 203, 205, 207; 209, 211, 213, 215, 217, 219, 221, 223, 225, 227, 229, 231, 233, 235, 237; 239, 241, 243, 245, 247, 249, 251; 253, 255, 257, 259, 261, 263, 265; 267, 269, 271, 273, 275, 277, 279, 281, 283; 285, 287, 289, 291; 293, 295, 297, 299, 301, 303, 305, 307; 309, 311, 313, 315, 317, 319, 321; 323, 325; 327, 329 and/or 331.

4.-35. (canceled)

36. The recombinant DNA construct of claim 1, wherein the plant-expressible promoter is a vascular promoter.

37. The recombinant DNA construct of claim 36, wherein the vascular promoter comprises one of the following: a sucrose synthase promoter, a sucrose transporter promoter, a corn sucrose synthase-1 (Sh1) promoter, Commelina yellow mottle virus (CoYMV) promoter, a wheat dwarf geminivirus (WDV) large intergenic region (LIR) promoter, a maize streak geminivirus 15 (MSV) coat protein (CP) promoter, a rice yellow stripe 1 (YS1)-like promoter, or a rice yellow stripe 2 (OsYSL2) promoter.

38. The recombinant DNA construct of claim 36, wherein the vascular promoter comprises a DNA sequence that is at least 80%, at least 85%, at least 90%, at least 95%, at least 96%, at least 97%, at least 98%, at least 99%, at least 99.5% or 100% identical to one or more of SEQ ID NOs: 335, 336, 337, 338, 339, or a functional portion thereof.

39. The recombinant DNA construct of claim 1, wherein the plant-expressible promoter is a rice tungro bacilliform virus (RTBV) promoter.

40. The recombinant DNA construct of claim 39, wherein the plant-expressible promoter comprises a DNA sequence that is at least 80%, at least 85%, at least 90%, at least 95%, at least 96%, at least 97%, at least 98%, at least 99%, at least 99.5% or 100% identical to one or more of SEQ ID NOs: 333, 334, or a functional portion thereof.

41. The recombinant DNA construct of claim 1, wherein the plant-expressible promoter is a leaf promoter.

42. The recombinant DNA construct of claim 41, wherein the leaf promoter comprises one of the following: a ribulose biphosphate carboxylase (RuBisCO) promoter, a pyruvate phosphate dikinase (PPDK) promoter, a fructose 1,6 bisphosphate adolase (FDA)promoter, a Nadh Gogat promoter, a chlorophyll a/b binding protein gene promoter, a phosphoenolpyruvate carboxylase (PEPC) promoter, or a Myb gene promoter.

43. The recombinant DNA construct of claim 41, wherein the leaf promoter comprises a DNA sequence that is at least 80%, at least 85%, at least 90%, at least 95%, at least 96%, at least 97%, at least 98%, at least 99%, at least 99.5% or 100% identical to one or more of SEQ ID NOs: 340, 341, 342, or a functional portion thereof.

44. The recombinant DNA construct of claim 1, wherein the plant-expressible promoter is a constitutive promoter.

45. The recombinant DNA construct of claim 44, wherein the constitutive promoter is selected from the group consisting of: an actin promoter, a CaMV 35S promoter, a CaMV 19S promoter, a plant ubiquitin promoter, a plant Gos2 promoter, a FMV promoter, a CMV promoter, a MMV promoter, a PCLSV promoter, an Emu promoter, a tubulin promoter, a nopaline synthase (nos) promoter, an octopine synthase (ocs) promoter, a mannopine synthase (mas) promoter, a maize alcohol dehydrogenase (Adh1) promoter, and a functional portion thereof.

46. The recombinant DNA construct of claim 44, wherein
the constitutive promoter comprises a DNA sequence that is at least 80%, at least 85%, at least 90%, at least 95%, at least 96%, at least 97%, at least 98%, at least 99%, at least 99.5% or 100% identical to one or more of SEQ ID NOs: 343, 344, 345, 346, 347, 348, 349, 350, 351, or a functional portion thereof.

47. A DNA molecule or vector comprising the recombinant DNA construct of claim 1.

48. A transgenic corn plant, plant part or plant cell comprising the recombinant DNA construct of claim 1 stably integrated into the genome of the transgenic corn plant, plant part or plant cell.

49. The transgenic corn plant of claim 48, wherein the transgenic corn plant has one or more of the following traits relative to a control plant: shorter plant height, increased stalk/stem diameter, improved lodging resistance, reduced green snap, deeper roots, increased leaf area, earlier canopy closure, higher stomatal conductance, lower ear height, increased foliar water content, improved drought tolerance, improved nitrogen use efficiency, reduced anthocyanin content and area in leaves under normal or nitrogen-limiting or water-limiting stress conditions, increased ear weight, increased harvest index, increased yield, increased seed number, increased seed weight, and/or increased prolificacy.

50. The transgenic corn plant of claim 49, wherein the transgenic corn plant has a shorter plant height and/or improved lodging resistance.

51. The transgenic corn plant of claim 48, wherein the height of the transgenic corn plant is at least 10%, at least 20%, at least 25%, at least 30%, at least 35%, or at least 40% shorter than a wild-type control plant.

52. The transgenic corn plant of claim 48, wherein the stalk or stem diameter of the transgenic corn plant at one or more stem internodes is at least 5%, at least 10%, at least 15%, at least 20%, at least 25%, at least 30%, at least 35%, or at least 40% greater than the stalk or stem diameter at the same one or more internodes of a wildtype control plant.

53. The transgenic corn plant of claim 48, wherein the stalk or stem diameter of the transgenic corn plant at one or more of the first, second, third, and/or fourth internode below the ear is at least 5%, at least 10%, at least 15%, at least 20%, at least 25%, at least 30%, at least 35%, or at least 40% greater than the same internode of a wild-type control plant.

54. The transgenic corn plant of claim 48, wherein the level of one or more active GAs in at least one internode tissue of the stem or stalk of the transgenic corn plant is lower than the same internode tissue of a wild-type control plant.

55. The transgenic corn plant of claim 48, wherein the level of one or more active GAs in at least one internode tissue of the stem or stalk of the transgenic corn plant is at least 5%, at least 10%, at least 15%, at least 20%, at least 25%, at least 30%, at least 35%, or at least 40% lower than the same internode tissue of a wild-type control plant.

56. The transgenic corn plant of claim 48, wherein the transgenic corn plant does not have any significant off-types in at least one female organ or ear.

57. A bacterial or host cell comprising the recombinant DNA construct of claim 1.

58. A method for producing a transgenic corn plant, comprising:
(a) transforming at least one cell of an explant with the recombinant DNA construct of claim 1, and
(b) regenerating or developing the transgenic corn plant comprising the recombinant DNA construct from the transformed explant.

59. The method of claim 58, wherein the at least one cell of an explant is transformed via Agrobacterium mediated transformation or particle bombardment.

60. The method of claim 58, wherein the at least one cell of an explant is transformed via site-directed integration using a targeted genome editing technique.

61. The method of claim 60, wherein the at least one cell of an explant is transformed using a recombinant DNA donor template comprising at least one homology arm and an insertion sequence, wherein the at least one homology arm is complementary to a target site in the genome of a corn plant, wherein the insertion sequence comprises a recombinant DNA construct comprising a transcribable DNA sequence encoding a GA2 oxidase protein, wherein the transcribable DNA sequence is operably linked to a plant-expressible promoter, and wherein the insertion sequence comprising the recombinant DNA construct is stably integrated into the genome of the transgenic corn plant.

62. (canceled)

63. The method of claim 61, wherein the at least one homology arm is at least 70%, at least 75%, at least 80%, at least 85%, at least 90%, at least 95%, at least 96%, at least 97%, at least 99% or 100% complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a target site in the genome of a corn plant.

64. The method of claim 58, wherein the at least one cell of an explant is transformed using a site-specific nuclease.

65. The method of claim 64, wherein the site-specific nuclease is a meganuclease, a zinc finger nuclease (ZFN), an RNA-guided endonuclease, a TALE-endonuclease (TALEN), a recombinase, or a transposase.

66. The method of claim 64, wherein the site-specific nuclease is an RNA-guided endonuclease.

67. The method of claim 66, wherein the at least one cell of an explant is further transformed using a guide RNA (gRNA) molecule.

68. The method of claim 67, wherein the RNA-guided endonuclease in association with the guide RNA molecule causes a double strand break or nick at or near a target DNA sequence of the guide RNA in the genome of the corn plant to direct the integration of the insertion sequence into the genome of the transgenic corn plant at or near the target DNA sequence of the guide RNA.

69. The method of claim 67, wherein the guide RNA molecule is a CRISPR RNA (crRNA) or a single-chain guide RNA (sgRNA).

70. The method of claim 68, wherein the guide RNA comprises a sequence complementary to a protospacer adjacent motif (PAM) sequence present in the genome of the transgenic corn plant immediately adjacent to the target DNA sequence of the guide RNA.

71. (canceled)

72. The method of claim 58, further comprising:
(c) selecting a transgenic corn plant comprising the recombinant DNA construct.

73. The method of claim 72, wherein the selecting step (c) comprises determining if the recombinant DNA construct was transformed or integrated into the genome of the transgenic corn plant using a molecular assay.

74. The method of claim 72, wherein the selecting step (c) comprises determining if the recombinant DNA construct was transformed or integrated into the genome of the transgenic corn plant by observing a plant phenotype.

75. A recombinant DNA donor template comprising at least one homology arm and an insertion sequence, wherein the at least one homology arm is at least 70%, at least 75%, at least 80%, at least 85%, at least 90%, at least 95%, at least 96%, at least 97%, at least 99% or 100% complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a target site in the genome of a corn plant, wherein the insertion sequence comprises a recombinant DNA construct comprising a transcribable DNA sequence encoding a GA2 oxidase protein and a plant expressible promoter, and wherein the transcribable DNA sequence is operably linked to the plant expressible promoter.

76. The recombinant DNA donor template of claim 75, wherein the at least one homology arm comprises two homology arms including a first homology arm and a second homology arm, wherein the first homology arm comprises a sequence that is at least 70%, at least 75%, at least 80%, at least 85%, at least 90%, at least 95%, at least 96%, at least 97%, at least 99% or 100% complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a first flanking DNA sequence, and the second homology arm comprises a sequence that is at least 70%, at least 75%, at least 80%, at least 85%, at least 90%, at least 95%, at least 96%, at least 97%, at least 99% or 100% complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a second flanking DNA sequence, wherein the first flanking DNA sequence and the second flanking DNA sequence are genomic sequences at or near the same genomic locus of a corn plant, and wherein the insertion sequence is located between the first homology arm and the second homology arm.

77. A DNA molecule or vector comprising the recombinant DNA donor template of claim 75.

78. The DNA molecule or vector of claim 77, further comprising a polynucleotide sequence encoding a site-specific nuclease

79. The DNA molecule or vector of claim 77, further comprising a polynucleotide sequence encoding a guide RNA.

80. A composition comprising the DNA molecule or vector of claim 77 and a guide RNA.

81. A composition comprising the DNA molecule or vector of claim 77 and a site-specific nuclease.

82. The composition of claim 81, further comprising a guide RNA.

83. A composition comprising a first DNA molecule or vector and a second DNA molecule or vector, wherein the first DNA molecule or vector comprises the recombinant DNA donor template of claim 75 and the second DNA molecule or vector comprises a polynucleotide sequence encoding a site-specific nuclease.

84. The composition of claim 83, wherein the first DNA molecule or vector or the second DNA molecule or vector comprises a polynucleotide sequence encoding a guide RNA.

85. A composition comprising a first DNA molecule or vector and a second DNA molecule or vector, wherein the first DNA molecule or vector comprises the recombinant DNA donor template of claim 75 and the second DNA molecule or vector comprises a polynucleotide sequence encoding a guide RNA.

86. A transgenic corn plant, plant part or plant cell comprising the insertion sequence of the recombinant DNA donor template of claim 75.
